SEQUENCE LISTING (212 sequences)

SEQ ID NO: 1 - DNA, 50 bp, Homo sapiens
  source 1..50: mol_type = unassigned DNA
ggcggactcgggactgctgctaaagcggggttcctgccgttccacttggc

SEQ ID NO: 2 - DNA, 50 bp, Homo sapiens
  source 1..50: mol_type = unassigned DNA
gggtggcagacccagatcctgaggtcctgcggcttcctccccgggggagc

SEQ ID NO: 3 - DNA, 50 bp, Homo sapiens
  source 1..50: mol_type = unassigned DNA
ggcggctgggactggaggacagcggtggcggaggcgactagcggcggcgg

SEQ ID NO: 4 - DNA, 50 bp, Homo sapiens
  source 1..50: mol_type = unassigned DNA
gctcgctcgctccctccctccgctggtgcgtttagtcagtcagccagcag

SEQ ID NO: 5 - DNA, 50 bp, Homo sapiens
  source 1..50: mol_type = unassigned DNA
gcttccttctttatttttctctacagctctcctcaccatctgacacatac

SEQ ID NO: 6 - DNA, 50 bp, Homo sapiens
  source 1..50: mol_type = unassigned DNA
ttttacaagccatttctgagatgggaaactattaggtcagtgcaaaagtg

SEQ ID NO: 7 - DNA, 50 bp, Homo sapiens
  source 1..50: mol_type = unassigned DNA
gtctaagaaatatttttgtgctttctggccttcttttcccaccttaggct

SEQ ID NO: 8 - DNA, 50 bp, Homo sapiens
  source 1..50: mol_type = unassigned DNA
acttccggcggcggctgaggcggcggccgaggagcggcggactcggggcg

SEQ ID NO: 9 - DNA, 50 bp, Homo sapiens
  source 1..50: mol_type = unassigned DNA
tcaaacaaaatactggttttgttttactgagaggcactgtgggtttttgt

SEQ ID NO: 10 - DNA, 50 bp, Homo sapiens
  source 1..50: mol_type = unassigned DNA
agcggcctctgacaccagcacagcaaacccgccgggatcaaagtgtacca

SEQ ID NO: 11 - DNA, 50 bp, Homo sapiens
  source 1..50: mol_type = unassigned DNA
cggtagggccgctgtatctgggagtaggggactaagagtctgagggtcca

SEQ ID NO: 12 - DNA, 50 bp, Homo sapiens
  source 1..50: mol_type = unassigned DNA
tactgaccaccctcacccatgaggctttctggatcattctctgaacttta

SEQ ID NO: 13 - DNA, 50 bp, Homo sapiens
  source 1..50: mol_type = unassigned DNA
ttgtacctttctctcctcgactgtgaagcgggccgggacctgccaggcca

SEQ ID NO: 14 - DNA, 50 bp, Homo sapiens
  source 1..50: mol_type = unassigned DNA
atggggctggctggttgtgggatctggaggcatctggggttggaatgtga

SEQ ID NO: 15 - DNA, 50 bp, Homo sapiens
  source 1..50: mol_type = unassigned DNA
ggctgcttgaagtcccgggagtcggtgaggcggctgcaggtccctccctg

SEQ ID NO: 16 - DNA, 50 bp, Homo sapiens
  source 1..50: mol_type = unassigned DNA
ctgtatatgtttctggagtcctgagcctgagctaaacaaaagcaggaggc

SEQ ID NO: 17 - DNA, 50 bp, Homo sapiens
  source 1..50: mol_type = unassigned DNA
tctggaagaagtaatttgctctatggttctctggtgtcctgaaaagtgta

SEQ ID NO: 18 - DNA, 50 bp, Homo sapiens
  source 1..50: mol_type = unassigned DNA
ggttcacagcgggcagggaaagccgcgggaagggtactccaggcgagagg

SEQ ID NO: 19 - DNA, 50 bp, Homo sapiens
  source 1..50: mol_type = unassigned DNA
tggcggagcgaacgggaccggcccggcttcagagcgcgaggtggagggtg

SEQ ID NO: 20 - DNA, 50 bp, Homo sapiens
  source 1..50: mol_type = unassigned DNA
gcaggaggaaactgctcgggctgcaagcagtcttccaggctttgcggctg

SEQ ID NO: 21 - DNA, 50 bp, Homo sapiens
  source 1..50: mol_type = unassigned DNA
cgcaagctgggttggcagaggacgctggaacctggctggtcggggagaaa

SEQ ID NO: 22 - DNA, 50 bp, Homo sapiens
  source 1..50: mol_type = unassigned DNA
tgaccacaacatggctgcggcgcctgggctgctcgtctggctgctcgtgc

SEQ ID NO: 23 - DNA, 150 bp, Homo sapiens
  source 1..150: mol_type = unassigned DNA
ggaacatctgaatgctgaggcctagagatcgttcagggtgttgtaactgggaacatctgaatgctgaggcctagagatcgttcagggtgttgtaactgggaacatctgaatgctgaggcctagagatcgttcagggtgttgtaactggga

SEQ ID NO: 24 - DNA, 50 bp, Homo sapiens
  source 1..50: mol_type = unassigned DNA
tccggagcgcgtggcgggcgtcagcgcggtggccagcgcgcagaggcggg

SEQ ID NO: 25 - DNA, 50 bp, Homo sapiens
  source 1..50: mol_type = unassigned DNA
gcgtttctgtttggagagactcagccatcatgccagacccgtccaaatcg

SEQ ID NO: 26 - DNA, 50 bp, Homo sapiens
  source 1..50: mol_type = unassigned DNA
tgtttgcacaaatttccttaaaaatcaacttgtactgtagcataagaaaa

SEQ ID NO: 27 - DNA, 50 bp, Homo sapiens
  source 1..50: mol_type = unassigned DNA
ctttatggcactggtagacaaaactatcaactgtgttaaaataattctag

SEQ ID NO: 28 - DNA, 50 bp, Homo sapiens
  source 1..50: mol_type = unassigned DNA
ctgcccttatcagttttgacctgctaggtgcttcacagaactttgcttga

SEQ ID NO: 29 - DNA, 150 bp, Homo sapiens
  source 1..150: mol_type = unassigned DNA
tcatcatattatacagaccgaactgttgtataaatttatttactgctagtcttaagaactgctttctttcgtttgtttgtttcaatattttccttctctctcaatttttggttgaataaactagattacattcagttggcctaaggtggt

SEQ ID NO: 30 - DNA, 50 bp, Homo sapiens
  source 1..50: mol_type = unassigned DNA
ccagcctgagcaacatagcgagaccccgtgtctctttttttttttttttt

SEQ ID NO: 31 - DNA, 50 bp, Homo sapiens
  source 1..50: mol_type = unassigned DNA
gtgggaggatcacttgagcccggaagttcaagaccaccctaggcaatata

SEQ ID NO: 32 - DNA, 50 bp, Homo sapiens
  source 1..50: mol_type = unassigned DNA
atgaatctaagagagaatggaatgtatgggaaaagaaagttactggaact

SEQ ID NO: 33 - DNA, 50 bp, Homo sapiens
  source 1..50: mol_type = unassigned DNA
aataaatatgcattctcttattggagcaggcagccaggaatgtagagctc

SEQ ID NO: 34 - DNA, 50 bp, Homo sapiens
  source 1..50: mol_type = unassigned DNA
ggactcacgtggatgacagtggaggcctcctggatctctaggtctcaggg

SEQ ID NO: 35 - DNA, 50 bp, Homo sapiens
  source 1..50: mol_type = unassigned DNA
agacacctgcgggagggaccagagcccggtcagggcgagggggcggaggc

SEQ ID NO: 36 - DNA, 50 bp, Homo sapiens
  source 1..50: mol_type = unassigned DNA
tgtcgatttcctgtagtgaatcaggcaccggagtgcaggttcgggggtgg

SEQ ID NO: 37 - DNA, 50 bp, Homo sapiens
  source 1..50: mol_type = unassigned DNA
ccccccgccactgctgaatttgactggctataaaaataaaataaaatcca

SEQ ID NO: 38 - DNA, 50 bp, Homo sapiens
  source 1..50: mol_type = unassigned DNA
tccttgatgtataaaaattaacaaaaataattttacttgtggcaatgttc

SEQ ID NO: 39 - DNA, 350 bp, Homo sapiens
  source 1..350: mol_type = unassigned DNA
cactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctg

SEQ ID NO: 40 - DNA, 50 bp, Homo sapiens
  source 1..50: mol_type = unassigned DNA
actttttttttttttttttagcagtttgagttggtgtagtgtattcttgg

SEQ ID NO: 41 - DNA, 100 bp, Homo sapiens
  source 1..100: mol_type = unassigned DNA
tatgaggagctgcagagcctggctgggaagcacggggatgacctgcggcgcacaaagactgagatctctgagatgaaccggaacatcagccggctccagg

SEQ ID NO: 42 - DNA, 50 bp, Homo sapiens
  source 1..50: mol_type = unassigned DNA
tggacccaaactgaggagcccggagctgccgctgggggatcggggccggg

SEQ ID NO: 43 - DNA, 50 bp, Homo sapiens
  source 1..50: mol_type = unassigned DNA
agggccgggcgggacgggaaacgttagggcagcggcccccggggtgaggg

SEQ ID NO: 44 - DNA, 50 bp, Homo sapiens
  source 1..50: mol_type = unassigned DNA
cgtagccccagctgaggcaggagaattgcttgagcccaggaactggaggc

SEQ ID NO: 45 - DNA, 50 bp, Homo sapiens
  source 1..50: mol_type = unassigned DNA
gccagcccagaacactggtctcgggcccgagaagacctcctttttccagg

SEQ ID NO: 46 - DNA, 50 bp, Homo sapiens
  source 1..50: mol_type = unassigned DNA
cttgttacaactgagtccgggttggaggaggtgtggggccgctgccgcca

SEQ ID NO: 47 - DNA, 50 bp, Homo sapiens
  source 1..50: mol_type = unassigned DNA
cccccacaaccgcgcctggctactttttgtatttttagtagagacagggt

SEQ ID NO: 48 - DNA, 50 bp, Homo sapiens
  source 1..50: mol_type = unassigned DNA
aatgtcaagttagaaaaaatgttgtagtttcatgttgttggtttgcaaat

SEQ ID NO: 49 - DNA, 50 bp, Homo sapiens
  source 1..50: mol_type = unassigned DNA
tgggtctgaattccagtctatcacttgttatgtgacctcggatgaatcac

SEQ ID NO: 50 - DNA, 50 bp, Homo sapiens
  source 1..50: mol_type = unassigned DNA
tgaaatgcttaggaccaaaagtgtttcagatttcagatttttaaaatatt

SEQ ID NO: 51 - DNA, 50 bp, Homo sapiens
  source 1..50: mol_type = unassigned DNA
taagccgtggaaaatgttcttgatcatttgcagttaaggactttaaataa

SEQ ID NO: 52 - DNA, 50 bp, Homo sapiens
  source 1..50: mol_type = unassigned DNA
ttgtcagtgccgcagccccgaccgccgggagctcgaacccgagccggggc

SEQ ID NO: 53 - DNA, 50 bp, Homo sapiens
  source 1..50: mol_type = unassigned DNA
ttgtgggtaagttctctctgagatttgggagattcataaatttgtgtttt

SEQ ID NO: 54 - DNA, 50 bp, Homo sapiens
  source 1..50: mol_type = unassigned DNA
cactcctgacatcatctgataaaagaaagtcttcagccgaatttaattta

SEQ ID NO: 55 - DNA, 50 bp, Homo sapiens
  source 1..50: mol_type = unassigned DNA
tgagccgcttggcgtcgtggtatctgagaagctgctctattcctttcctt

SEQ ID NO: 56 - DNA, 50 bp, Homo sapiens
  source 1..50: mol_type = unassigned DNA
ctcagctgcgcccagagccttcggccggacctgaaaaagcgagagggaga

SEQ ID NO: 57 - DNA, 50 bp, Homo sapiens
  source 1..50: mol_type = unassigned DNA
tctgtcacctgactcggctcaaacatggctgcgctgagagctctattgct

SEQ ID NO: 58 - DNA, 50 bp, Homo sapiens
  source 1..50: mol_type = unassigned DNA
gttctgagattcactactggtatggcctggagaatgcagaccctgtccag

SEQ ID NO: 59 - DNA, 50 bp, Homo sapiens
  source 1..50: mol_type = unassigned DNA
ccaaaactattcatttgcattgtagtagtttgtacagtcatatgtaaaac

SEQ ID NO: 60 - DNA, 50 bp, Homo sapiens
  source 1..50: mol_type = unassigned DNA
gtcggggaagcgttctaggctgggcgcgcggtctcgggtcaggtgtggcg

SEQ ID NO: 61 - DNA, 50 bp, Homo sapiens
  source 1..50: mol_type = unassigned DNA
tatgtcctggtgaggctccagcaagcagtgaagccccacctgtccatccc

SEQ ID NO: 62 - DNA, 50 bp, Homo sapiens
  source 1..50: mol_type = unassigned DNA
catgttttgttacaggttgtagagtatttgcagaaggaaaccatttctgg

SEQ ID NO: 63 - DNA, 50 bp, Homo sapiens
  source 1..50: mol_type = unassigned DNA
gggacacagcgggacaggtgagaagctgggcgcggcctctactggtctgc

SEQ ID NO: 64 - DNA, 50 bp, Homo sapiens
  source 1..50: mol_type = unassigned DNA
tttatcccactcctgacagtttatcccactcttgacacttccccttgagc

SEQ ID NO: 65 - DNA, 50 bp, Homo sapiens
  source 1..50: mol_type = unassigned DNA
acaccaggtctcctggccttgctgactcccagtccagggagatggaggct

SEQ ID NO: 66 - DNA, 50 bp, Homo sapiens
  source 1..50: mol_type = unassigned DNA
atatggtgacctcctgggagcgggggaccaccaggttgcctaaggatggg

SEQ ID NO: 67 - DNA, 250 bp, Homo sapiens
  source 1..250: mol_type = unassigned DNA
caccagcccggaggggcaggtctcggtctagaacacctgctaggcgcagatctaggacccgatcaccagtacgacgcaggtctcgtagtagatcaccagccaggagaagtggcaggtcacgctctagaaccccagctagacgtggccgctcacgctccagaaccccagccagacgtggccgctcacgctctagaaccccagctagacgcagtggtcgctcacgctccagaacaccagccaggagagggag

SEQ ID NO: 68 - DNA, 50 bp, Homo sapiens
  source 1..50: mol_type = unassigned DNA
tggctctgtacctggacagggctgcggtaggccagcggtgggctggcggt

SEQ ID NO: 69 - DNA, 50 bp, Homo sapiens
  source 1..50: mol_type = unassigned DNA
ctgcaatatctgattacattgatgaattccgttgtattgtatgtgtgaat

SEQ ID NO: 70 - DNA, 50 bp, Homo sapiens
  source 1..50: mol_type = unassigned DNA
attagaatgagtagtagtgtctgggtgcagtgactcatgccccattgaaa

SEQ ID NO: 71 - DNA, 50 bp, Homo sapiens
  source 1..50: mol_type = unassigned DNA
tgagcccaggaactggaggctgcagtgagctatgactgttccacttcact

SEQ ID NO: 72 - DNA, 50 bp, Homo sapiens
  source 1..50: mol_type = unassigned DNA
gtcgcagccaccggtcacctcagctgtgaaccatgtaggagccgtctggg

SEQ ID NO: 73 - DNA, 50 bp, Homo sapiens
  source 1..50: mol_type = unassigned DNA
cctcgtgaactcagaagttagttttcgtctctggacctttttataatcgg

SEQ ID NO: 74 - DNA, 50 bp, Homo sapiens
  source 1..50: mol_type = unassigned DNA
ccatcatgttctttattattgtgattgagacctttctgtgggccaggcat

SEQ ID NO: 75 - DNA, 50 bp, Homo sapiens
  source 1..50: mol_type = unassigned DNA
tgtcgggccgcggcggcgcttggcagccaggagctctgcattgaaggcac

SEQ ID NO: 76 - DNA, 50 bp, Homo sapiens
  source 1..50: mol_type = unassigned DNA
tgccaggggcactgccttctcacagctggccttgccccgtccaccctgtg

SEQ ID NO: 77 - DNA, 50 bp, Homo sapiens
  source 1..50: mol_type = unassigned DNA
gtgctactcgctcgaacccgccgggcgaaggaagcacttcccatgctctc

SEQ ID NO: 78 - DNA, 50 bp, Homo sapiens
  source 1..50: mol_type = unassigned DNA
agtggactcaggcatcaaagaagtggccaacgtgtgcaaccagagcctga

SEQ ID NO: 79 - DNA, 50 bp, Homo sapiens
  source 1..50: mol_type = unassigned DNA
attcagccgtatcagtgaagagtgaagcactgcactctttaaggataggg

SEQ ID NO: 80 - DNA, 100 bp, Homo sapiens
  source 1..100: mol_type = unassigned DNA
agcacaaatatggtgacctcctgggagcgggggaccaccaggttgcctaaggaggggtgaaccagggcaggtcggaaatggagcatgtcaaaactcccgc

SEQ ID NO: 81 - DNA, 50 bp, Homo sapiens
  source 1..50: mol_type = unassigned DNA
ccttggatccggcctcccgcccagtgcctttggtcgccggctgccgcacc

SEQ ID NO: 82 - DNA, 50 bp, Homo sapiens
  source 1..50: mol_type = unassigned DNA
ccctgagctgcccttgcgttcagttggaagccacgcaaaatgaactgaac

SEQ ID NO: 83 - DNA, 50 bp, Homo sapiens
  source 1..50: mol_type = unassigned DNA
gcgtgagaggcgcgcggcggcgcagtgaacagtctccttccacaaaacca

SEQ ID NO: 84 - DNA, 50 bp, Homo sapiens
  source 1..50: mol_type = unassigned DNA
gcagccctggcagtgcctccggcgctttgccctggcctggtgggagagga

SEQ ID NO: 85 - DNA, 50 bp, Homo sapiens
  source 1..50: mol_type = unassigned DNA
tgacacctagcggagcgatgcccaaccaggcgcagatgcggatcctgaaa

SEQ ID NO: 86 - DNA, 100 bp, Homo sapiens
  source 1..100: mol_type = unassigned DNA
tcccctggacccgcccccatctgcccaagataattttagtttccttgggcctggaatctggacacacagggctcccccccgcctctgacttctctgtccg

SEQ ID NO: 87 - DNA, 50 bp, Homo sapiens
  source 1..50: mol_type = unassigned DNA
acatcctggtggtagaggggaagggaggaggtcagacctgtcactctcct

SEQ ID NO: 88 - DNA, 50 bp, Homo sapiens
  source 1..50: mol_type = unassigned DNA
ccaaaatggcgatgcctaccacctagaactggattgtgcggtaggcttaa

SEQ ID NO: 89 - DNA, 50 bp, Homo sapiens
  source 1..50: mol_type = unassigned DNA
taaccggctggccgggcggagctggcagcatttgattgtggcttgggacg

SEQ ID NO: 90 - DNA, 50 bp, Homo sapiens
  source 1..50: mol_type = unassigned DNA
gaatcctgaacaaacaatctgatctagctttggcctctctgctccccaat

SEQ ID NO: 91 - DNA, 50 bp, Homo sapiens
  source 1..50: mol_type = unassigned DNA
cgccacactctgggcacggcgtgggctgggacatttggaataaacggatc

SEQ ID NO: 92 - DNA, 50 bp, Homo sapiens
  source 1..50: mol_type = unassigned DNA
cgcgcgtcggccgcgctcagcggcagagcggagcggagctgtgaggcgcc

SEQ ID NO: 93 - DNA, 50 bp, Homo sapiens
  source 1..50: mol_type = unassigned DNA
tagtgtgcggagaggggcgcggggtggaattgctgcgggaagtgggaaac

SEQ ID NO: 94 - DNA, 50 bp, Homo sapiens
  source 1..50: mol_type = unassigned DNA
gcggctgaaggcgatccgcagtgaggccccagccattcggattgagcctt

SEQ ID NO: 95 - DNA, 50 bp, Homo sapiens
  source 1..50: mol_type = unassigned DNA
gcagcgctggaggaagaattcacgttgtcttcggtagtcctgagcgccgg

SEQ ID NO: 96 - DNA, 50 bp, Homo sapiens
  source 1..50: mol_type = unassigned DNA
ggcctgtcgggtcagggcggttcgcgggtgctgtcagagctgggccgggg

SEQ ID NO: 97 - DNA, 50 bp, Homo sapiens
  source 1..50: mol_type = unassigned DNA
atggctgcccccgcagtgaaggttgcccgaggatggtcgggcctggcgtt

SEQ ID NO: 98 - DNA, 50 bp, Homo sapiens
  source 1..50: mol_type = unassigned DNA
gggcagaggcagtgtgggctgatgatgtgctttggccttctcgggactgt

SEQ ID NO: 99 - DNA, 50 bp, Homo sapiens
  source 1..50: mol_type = unassigned DNA
ctggcctggcaggtgacggtgctggatgtggcctttttgccttttctaaa

SEQ ID NO: 100 - DNA, 50 bp, Homo sapiens
  source 1..50: mol_type = unassigned DNA
tttccttaacattcttcctcccctgcctggcccagtccttatcccctgct

SEQ ID NO: 101 - DNA, 200 bp, Homo sapiens
  source 1..200: mol_type = unassigned DNA
aagaaaatagacaatacacttatctggctggggagataccatgatcatgaaggtggttctcagagtgaggctcatccattgcactttggttgtgctgacccctgtgatttccccaaatgcagaatttgtagtagtgagggactgtgttcgtgctttcccctgtcattttttgtcctaagagatcatagtgtgaagtttat

SEQ ID NO: 102 - DNA, 100 bp, Homo sapiens
  source 1..100: mol_type = unassigned DNA
actgccatgaagctctgcagaagaaagatctggaagtgggagacactttcactatatatagtggctcccacttccagatctttctctctgtatatatagt

SEQ ID NO: 103 - DNA, 50 bp, Homo sapiens
  source 1..50: mol_type = unassigned DNA
cattggtgattaaggcttcaacatacaaatttcgggggggacaaaaacat

SEQ ID NO: 104 - DNA, 50 bp, Homo sapiens
  source 1..50: mol_type = unassigned DNA
gcggccgccacgcgacgcctggctgggcccgcaccggagaggcgtctcgg

SEQ ID NO: 105 - DNA, 50 bp, Homo sapiens
  source 1..50: mol_type = unassigned DNA
ccctttcgccacagagcgcgcggaggacaaggtgaccagaggttccccag

SEQ ID NO: 106 - DNA, 50 bp, Homo sapiens
  source 1..50: mol_type = unassigned DNA
agttgcacggcggcgtgtgcgtttcctagttgtctggtgctgctatatag

SEQ ID NO: 107 - DNA, 50 bp, Homo sapiens
  source 1..50: mol_type = unassigned DNA
tcagacatgacattcagactgaggtcctcaaaactgaggggcattttctg

SEQ ID NO: 108 - DNA, 50 bp, Homo sapiens
  source 1..50: mol_type = unassigned DNA
aggctgtaaagagggcgctacccagaggtgcatctgcaggaaaagcccac

SEQ ID NO: 109 - DNA, 50 bp, Homo sapiens
  source 1..50: mol_type = unassigned DNA
atataggaaagaaccacaggtagtttcatgtggttggaggacagagggaa

SEQ ID NO: 110 - DNA, 50 bp, Homo sapiens
  source 1..50: mol_type = unassigned DNA
ggcaggcgggccgcggcggcggcgggcagcggacgggcggactgacgggc

SEQ ID NO: 111 - DNA, 50 bp, Homo sapiens
  source 1..50: mol_type = unassigned DNA
agtcctagctacttgagagactcagctgggatgatcacttgagcccagga

SEQ ID NO: 112 - DNA, 50 bp, Homo sapiens
  source 1..50: mol_type = unassigned DNA
agggcagctcgccccgcggagtccgggctgaaccacactgcgcggccgcg

SEQ ID NO: 113 - DNA, 50 bp, Homo sapiens
  source 1..50: mol_type = unassigned DNA
aggtagtgagttatctcaattgattgttcgctttcagttacagattaagc

SEQ ID NO: 114 - DNA, 50 bp, Homo sapiens
  source 1..50: mol_type = unassigned DNA
tgctgttctccaaccagtgcttatgctgcaggcaattttgttgttcagaa

SEQ ID NO: 115 - DNA, 50 bp, Homo sapiens
  source 1..50: mol_type = unassigned DNA
ctaatcagacgttgctgaagtattgtttttcaatgatgattgactgagaa

SEQ ID NO: 116 - DNA, 50 bp, Homo sapiens
  source 1..50: mol_type = unassigned DNA
ccttctgtcccctgctcccttgttccccagtcccctgtgtcatcaagatg

SEQ ID NO: 117 - DNA, 150 bp, Homo sapiens
  source 1..150: mol_type = unassigned DNA
ctcaccaagcaagtgtcgtggggcttgctggcttgcaccgtgactcactctcactaagcaagtgtcgtggggcttgctggcttgcaccgtgactcactctcactaagcaagtgtcgtggggcttgctggcttgcaccgtgactccctctc

SEQ ID NO: 118 - DNA, 50 bp, Homo sapiens
  source 1..50: mol_type = unassigned DNA
cgaggcgacagctcgggctctggagccgggaggcgagaacgaggagggag

SEQ ID NO: 119 - DNA, 50 bp, Homo sapiens
  source 1..50: mol_type = unassigned DNA
ttccgggctccgggctctgggtggcggcggctgtgagcggcggcactgcg

SEQ ID NO: 120 - DNA, 50 bp, Homo sapiens
  source 1..50: mol_type = unassigned DNA
gagtctgagatcagtctgggccacatagcgagaccccgtctctatgttaa

SEQ ID NO: 121 - DNA, 50 bp, Homo sapiens
  source 1..50: mol_type = unassigned DNA
gttcaagaccagccttggcaatatagcgagaccccgtctctacaaaaaca

SEQ ID NO: 122 - DNA, 50 bp, Homo sapiens
  source 1..50: mol_type = unassigned DNA
aaaagcatatatacctctgaccagtgacgtggaataggcatgagacgagt

SEQ ID NO: 123 - DNA, 50 bp, Homo sapiens
  source 1..50: mol_type = unassigned DNA
ttacaaatttgactcttgaatggcaaaataatgttagtatgtagaaggtt

SEQ ID NO: 124 - DNA, 150 bp, Homo sapiens
  source 1..150: mol_type = unassigned DNA
acgactcctggtttgccacaagcccggcctctgtagtgagagagctgtgactgcgtttccagctccttgaaggcagagagactcctgcctttcggggcgggcgggctggaacaaagacaccaacggggccacctcggaaagtctttttga

SEQ ID NO: 125 - DNA, 50 bp, Homo sapiens
  source 1..50: mol_type = unassigned DNA
atggcggccaccatgaagaaggcggtgagtggggagctcggggctctgga

SEQ ID NO: 126 - DNA, 100 bp, Homo sapiens
  source 1..100: mol_type = unassigned DNA
aacaccaagagcagctctgagatcatgctggccctacgcgaattgagtttctgtggcctaattggatttggagaacgccttccctggccccttttcctca

SEQ ID NO: 127 - DNA, 50 bp, Homo sapiens
  source 1..50: mol_type = unassigned DNA
aacggcgcgccggctgtggccggcgcagagtagtgctcgggccgggggtc

SEQ ID NO: 128 - DNA, 50 bp, Homo sapiens
  source 1..50: mol_type = unassigned DNA
tgagattggtagacatatgacactggtagaattagtttgaacatctgtgt

SEQ ID NO: 129 - DNA, 50 bp, Homo sapiens
  source 1..50: mol_type = unassigned DNA
tgtgggtcctctgctctggcaacaggcagagcactgctttagagcctctg

SEQ ID NO: 130 - DNA, 50 bp, Homo sapiens
  source 1..50: mol_type = unassigned DNA
ttggagagtgcatccggcccggtacttgtgatcggaggagagccggatcg

SEQ ID NO: 131 - DNA, 50 bp, Homo sapiens
  source 1..50: mol_type = unassigned DNA
tggcgccagaactagtggcgggctgaggacgccgtacccctcggaaggca

SEQ ID NO: 132 - DNA, 50 bp, Homo sapiens
  source 1..50: mol_type = unassigned DNA
cttggcaacatagcgagaccccgtctctacaaaaaaatttaaaaattagc

SEQ ID NO: 133 - DNA, 50 bp, Homo sapiens
  source 1..50: mol_type = unassigned DNA
ccacgaggactttaaagaagacctgaagaagttccgcaccaagagccgga

SEQ ID NO: 134 - DNA, 50 bp, Homo sapiens
  source 1..50: mol_type = unassigned DNA
aggctgctaagtcatcctggcagcattgccacatgagccccatgcggtgc

SEQ ID NO: 135 - DNA, 50 bp, Homo sapiens
  source 1..50: mol_type = unassigned DNA
gtgaagatgctgctggaattgtccgaggagcataaggaacacctggcctt

SEQ ID NO: 136 - DNA, 50 bp, Homo sapiens
  source 1..50: mol_type = unassigned DNA
tgagctgtgggacgtgcacccaggactcggctcacacatgcagttcgctt

SEQ ID NO: 137 - DNA, 50 bp, Homo sapiens
  source 1..50: mol_type = unassigned DNA
cactgctgtagatgggcggtctgcgagcggagttaccgagttttactccg

SEQ ID NO: 138 - DNA, 50 bp, Homo sapiens
  source 1..50: mol_type = unassigned DNA
ccctggatgcctgccccttgaatgggggtcaggctgtgcatacattgtga

SEQ ID NO: 139 - DNA, 50 bp, Homo sapiens
  source 1..50: mol_type = unassigned DNA
cagtctgcgcagggactggcgggactgcgcggcggcgactacagacgtgt

SEQ ID NO: 140 - DNA, 250 bp, Homo sapiens
  source 1..250: mol_type = unassigned DNA
gggatgtgagggcgatctggctgcgacatctgtcacgccattgattgccagggttgattcggttgatcttgctggctagacgggtgtccccttcctccctcactgctccacatgcgtccctcccaaagctgcatgctccattgaccatccccaacagaggaggaccggtcttcggtcaagggtatatgagtagctgcactcccctgctagaacctccaaacaagctctcaaggtccaaatgacactgggg

SEQ ID NO: 141 - DNA, 50 bp, Homo sapiens
  source 1..50: mol_type = unassigned DNA
gcttaaaggaaagcaaaagctgtgttgagagaataaaacagggataagtc

SEQ ID NO: 142 - DNA, 50 bp, Homo sapiens
  source 1..50: mol_type = unassigned DNA
aaaatgcctatttaaattactatttcattatttttctcaaaagtgtgaaa

SEQ ID NO: 143 - DNA, 50 bp, Homo sapiens
  source 1..50: mol_type = unassigned DNA
cccaggaactggaggctgcagtgagctatgattgcaattacactccagcc

SEQ ID NO: 144 - DNA, 50 bp, Homo sapiens
  source 1..50: mol_type = unassigned DNA
tggggagaggaggcgagaggctctccttccccgcttcccccctaggggtt

SEQ ID NO: 145 - DNA, 50 bp, Homo sapiens
  source 1..50: mol_type = unassigned DNA
gactgcagtcctgagaccctataacctgtatgactagagaagtgaaacta

SEQ ID NO: 146 - DNA, 50 bp, Homo sapiens
  source 1..50: mol_type = unassigned DNA
gaaaaaccccccaaaccttaaaaatgtagaatctctcagctaatctatat

SEQ ID NO: 147 - DNA, 50 bp, Homo sapiens
  source 1..50: mol_type = unassigned DNA
cagggcttcggcctccggcgtcgggaaatggcggcggggggcaggatgga

SEQ ID NO: 148 - DNA, 50 bp, Homo sapiens
  source 1..50: mol_type = unassigned DNA
aaaaatgttagggtggtgcaaaagtgatcgtggtttttgcaattttttaa

SEQ ID NO: 149 - DNA, 50 bp, Homo sapiens
  source 1..50: mol_type = unassigned DNA
tattgtgtgatactgaggcttggagtgtgaatgaatccttcacccaggta

SEQ ID NO: 150 - DNA, 50 bp, Homo sapiens
  source 1..50: mol_type = unassigned DNA
tcggctcggtcctgaggagaaggactcagccgcggctgcgggacccgggc

SEQ ID NO: 151 - DNA, 50 bp, Homo sapiens
  source 1..50: mol_type = unassigned DNA
cgccgctgccgggttgccagcggagtcgcgcgtcgggagctacgtagggc

SEQ ID NO: 152 - DNA, 50 bp, Homo sapiens
  source 1..50: mol_type = unassigned DNA
cccctctatcaatgatgagactgatgctgagaaaagtaacatgattatgt

SEQ ID NO: 153 - DNA, 50 bp, Homo sapiens
  source 1..50: mol_type = unassigned DNA
tacacattaaagcagatctggagtctgaagtagctataaagcagctataa

SEQ ID NO: 154 - DNA, 50 bp, Homo sapiens
  source 1..50: mol_type = unassigned DNA
agaagcatgcctttcagggcattgataaaaagagtgaaatgttcaggacc

SEQ ID NO: 155 - DNA, 50 bp, Homo sapiens
  source 1..50: mol_type = unassigned DNA
ctgagcaagatgcaggatgacaatcaggtcatggtgtctgagggcatcat

SEQ ID NO: 156 - DNA, 50 bp, Homo sapiens
  source 1..50: mol_type = unassigned DNA
gcgcgttcccggcagctgcgggctccgaggccagagagaaaagactgcga

SEQ ID NO: 157 - DNA, 50 bp, Homo sapiens
  source 1..50: mol_type = unassigned DNA
gagcaacgcgactgaccgtggtcgtgggcggacggcggctgcagcgtgga

SEQ ID NO: 158 - DNA, 100 bp, Homo sapiens
  source 1..100: mol_type = unassigned DNA
ctggttttccgtctggtgaggggttacttccgggtcggacggcgctagctgcagcatcggagtgtggcagtgctgggctggccggcgggctgggctgcgg

SEQ ID NO: 159 - DNA, 50 bp, Homo sapiens
  source 1..50: mol_type = unassigned DNA
ctgcgcagaaacgctgatccggaaggcgctggctgagtcgattgcaggtc

SEQ ID NO: 160 - DNA, 50 bp, Homo sapiens
  source 1..50: mol_type = unassigned DNA
ctttcggcgggtgacatctttgctgagggctcaagcggagcgataggtca

SEQ ID NO: 161 - DNA, 50 bp, Homo sapiens
  source 1..50: mol_type = unassigned DNA
cttcgaaaatgagggtgaagatgaagccatgtttgtagaatatagaaaac

SEQ ID NO: 162 - DNA, 50 bp, Homo sapiens
  source 1..50: mol_type = unassigned DNA
ggagctgctggccaggccggagcgaggcagcgcgcccggctcccgcgcca

SEQ ID NO: 163 - DNA, 50 bp, Homo sapiens
  source 1..50: mol_type = unassigned DNA
tggcaggtctagtgtctttgccacttgcctggtgatttctatgatgaaat

SEQ ID NO: 164 - DNA, 150 bp, Homo sapiens
  source 1..150: mol_type = unassigned DNA
ggttacttctgtggacttgggccgaacagcactgtctaagcaggacatgaaaagaagggggaagcgtctctccttttcccttcatggaactttccattgaaaaattagccccttccagtccttctcggatgaagcacagttgccggttac

SEQ ID NO: 165 - DNA, 50 bp, Homo sapiens
  source 1..50: mol_type = unassigned DNA
cagcaactgtgataccttgtagaatatgagtgatatgcaagctgtgtttt

SEQ ID NO: 166 - DNA, 50 bp, Homo sapiens
  source 1..50: mol_type = unassigned DNA
aaccccaggccagggaggcccgggtttgggatgccttcctcgggagtgtg

SEQ ID NO: 167 - DNA, 50 bp, Homo sapiens
  source 1..50: mol_type = unassigned DNA
aggccacagccgctccctcgctctgctggggcctccggacgcgcttccca

SEQ ID NO: 168 - DNA, 150 bp, Homo sapiens
  source 1..150: mol_type = unassigned DNA
gttggtgttgaggtgagtccggtcccttttgcatccctaccccgacactgcgggttgtcacaacggcaccctcccgctttctctctgcctcggatttagtcgtgactgtgtgtctccgccgtggtgcagcttcaggcctctcccgcatct

SEQ ID NO: 169 - DNA, 50 bp, Homo sapiens
  source 1..50: mol_type = unassigned DNA
tgaagtgaggtaggaggttgatcaaattttctgtataacaggaatatgga

SEQ ID NO: 170 - DNA, 50 bp, Homo sapiens
  source 1..50: mol_type = unassigned DNA
tgaataataggcttatatgttataacatcaaaatataattcgagtttgac

SEQ ID NO: 171 - DNA, 50 bp, Homo sapiens
  source 1..50: mol_type = unassigned DNA
taagtgtaacattcagaaccgggtaacattcggcgaccgaacgcggcggt

SEQ ID NO: 172 - DNA, 50 bp, Homo sapiens
  source 1..50: mol_type = unassigned DNA
cacaaagtagggaatattcaagattgtattaggttggtgcaaaagtgatt

SEQ ID NO: 173 - DNA, 50 bp, Homo sapiens
  source 1..50: mol_type = unassigned DNA
ctgctggtttccaactttccgctcatcttcgtctccgcagcctcctgcaa

SEQ ID NO: 174 - DNA, 50 bp, Homo sapiens
  source 1..50: mol_type = unassigned DNA
gcagggacaacagtcagagggctgcaggggcctgaagccagacacgggac

SEQ ID NO: 175 - DNA, 50 bp, Homo sapiens
  source 1..50: mol_type = unassigned DNA
gaccggagggaggaggaggaggaagaagagcggagagagaaggaagaggc

SEQ ID NO: 176 - DNA, 50 bp, Homo sapiens
  source 1..50: mol_type = unassigned DNA
gactttggacatgaagtccccagcatctctaccagctccactgaattaag

SEQ ID NO: 177 - DNA, 100 bp, Homo sapiens
  source 1..100: mol_type = unassigned DNA
gctgtcggccgagcgcgccgtcaccggctacaccgacccctacaccgggcagcagatctccctcttccaggccatgcagaaggacctcatcgtccgggag

SEQ ID NO: 178 - DNA, 100 bp, Homo sapiens
  source 1..100: mol_type = unassigned DNA
tgctgtcggccgagcgcgccgtcaccggctacaccgacccctacaccgggcagcagatctccctcttccaggccatgcagaaggacctcatcgtccggga

SEQ ID NO: 179 - DNA, 100 bp, Homo sapiens
  source 1..100: mol_type = unassigned DNA
ctgtcggccgagcgcgccgtcaccggctacaccgacccctacaccgggcagcagatctccctcttccaggccatgcagaaggacctcatcgtccgggagc

SEQ ID NO: 180 - DNA, 100 bp, Homo sapiens
  source 1..100: mol_type = unassigned DNA
gtcggccgagcgcgccgtcaccggctacaccgacccctacaccgggcagcagatctccctcttccaggccatgcagaaggacctcatcgtccgggagcac

SEQ ID NO: 181 - DNA, 150 bp, Homo sapiens
  source 1..150: mol_type = unassigned DNA
agctgctgtcggccgagcgcgccgtcaccggctacaccgacccctacaccgggcagcagatctccctcttccaggccatgcagaaggacctcatcgtccgggagcacggcatccgcctgctggaggcccagatcgccacgggcggcgtca

SEQ ID NO: 182 - DNA, 100 bp, Homo sapiens
  source 1..100: mol_type = unassigned DNA
tcggccgagcgcgccgtcaccggctacaccgacccctacaccgggcagcagatctccctcttccaggccatgcagaaggacctcatcgtccgggagcacg

SEQ ID NO: 183 - DNA, 100 bp, Homo sapiens
  source 1..100: mol_type = unassigned DNA
ggagaaccggaagctgaccgtggaggaggcgttcaaagcaggaatgttcgggaaagaaacctacgtgaagctgctgtcggccgagcgcgccgtcaccggc

SEQ ID NO: 184 - DNA, 50 bp, Homo sapiens
  source 1..50: mol_type = unassigned DNA
ggcgagcgggcaggtgcggcgggtgcggcgggtgcggcgggtgcggcggg

SEQ ID NO: 185 - DNA, 100 bp, Homo sapiens
  source 1..100: mol_type = unassigned DNA
cagagatgcccctgctggccgcaaagtgggtctcattgctgcccgccggactggacgtctccggggaaccaagactgtgcaggagaaagagaactagtgc

SEQ ID NO: 186 - DNA, 50 bp, Homo sapiens
  source 1..50: mol_type = unassigned DNA
gggctccgagtcccgggagcggaggccggagtcgggttcctgtagaggct

SEQ ID NO: 187 - DNA, 50 bp, Homo sapiens
  source 1..50: mol_type = unassigned DNA
agacaagctcggtctgaggttgttttcctttgaactggctctctcacatt

SEQ ID NO: 188 - DNA, 50 bp, Homo sapiens
  source 1..50: mol_type = unassigned DNA
gagattgagaccagcctgggctaacatagcgagaccccgtctctacaaaa

SEQ ID NO: 189 - DNA, 50 bp, Homo sapiens
  source 1..50: mol_type = unassigned DNA
accacgccataggcccacacatcagactctgtagtgtagcggttataaaa

SEQ ID NO: 190 - DNA, 50 bp, Homo sapiens
  source 1..50: mol_type = unassigned DNA
cctctaaaaaacccattcccacactagctcggactgaggccaagataacc

SEQ ID NO: 191 - DNA, 50 bp, Homo sapiens
  source 1..50: mol_type = unassigned DNA
tgtagtggtcctgcaggagtggagcctgtaggacttgcttctcagcggct

SEQ ID NO: 192 - DNA, 50 bp, Homo sapiens
  source 1..50: mol_type = unassigned DNA
tgtcaggcagtggagttacttacagacaagagccttgctcaggccagccc

SEQ ID NO: 193 - DNA, 50 bp, Homo sapiens
  source 1..50: mol_type = unassigned DNA
tggaggctgcagttagctatgatcacaccactgcattccagcctgagtga

SEQ ID NO: 194 - DNA, 50 bp, Homo sapiens
  source 1..50: mol_type = unassigned DNA
ctaattgatcacaaccaattacagatttctttgtttcttctcctctccca

SEQ ID NO: 195 - DNA, 50 bp, Homo sapiens
  source 1..50: mol_type = unassigned DNA
cttttcctcattcgccagctttgtaggtgactgacctagtaggcatgtgg

SEQ ID NO: 196 - DNA, 50 bp, Homo sapiens
  source 1..50: mol_type = unassigned DNA
ctgggcaacatagcgagaccccgtctctctctagtgtgtgtgtgtgtgtg

SEQ ID NO: 197 - DNA, 50 bp, Homo sapiens
  source 1..50: mol_type = unassigned DNA
caggggagttgaggaggtttactgcaaacaactgttcttttttcttttgg

SEQ ID NO: 198 - DNA, 50 bp, Homo sapiens
  source 1..50: mol_type = unassigned DNA
gattctagaatcacaaataaagccaattaaaatctttaaatttgttgtac

SEQ ID NO: 199 - DNA, 50 bp, Homo sapiens
  source 1..50: mol_type = unassigned DNA
tggcacatctagcaacagagccagatcagaacccaggtaagctcggtctc

SEQ ID NO: 200 - DNA, 200 bp, Homo sapiens
  source 1..200: mol_type = unassigned DNA
accatggttgtctgagcatgcagcatgcttgtctgctcataccccatggtttctgagcaggaaccttcattgtctactgctttacagggaaatagtgttttatgcatcgtgtatatgagtttagtatttactcatattctatgactctctactcttagatcacttctgcctttttctgcacattgtttatctgttccaaa

SEQ ID NO: 201 - DNA, 50 bp, Homo sapiens
  source 1..50: mol_type = unassigned DNA
aacccaaaaagtcacacctgtgtcctgtgcgcgggtgctgcaggcttagg

SEQ ID NO: 202 - DNA, 20 bp, synthetic construct
  misc_feature 1..20: note = Description of Artificial Sequence: Synthetic primer
  source 1..20: mol_type = other DNA
ccagtgcagggtccgaggta

SEQ ID NO: 203 - DNA, 19 bp, synthetic construct
  misc_feature 1..19: note = Description of Artificial Sequence: Synthetic primer
  source 1..19: mol_type = other DNA
cccaggactcggctcacac

SEQ ID NO: 204 - DNA, 21 bp, Homo sapiens
  source 1..21: mol_type = unassigned DNA
caggactcggctcacacatgc

SEQ ID NO: 205 - DNA, 23 bp, Homo sapiens
  source 1..23: mol_type = unassigned DNA
ttgtctaaccctaactgagaagg

SEQ ID NO: 206 - DNA, 21 bp, synthetic construct
  misc_feature 1..21: note = Description of Artificial Sequence: Synthetic oligonucleotide
  source 1..21: mol_type = other DNA
agacgacagctggatcacacg

SEQ ID NO: 207 - RNA, 21 nt, synthetic construct
  misc_feature 1..21: note = Description of Artificial Sequence: Synthetic oligonucleotide
  modified_base 1..2: mod_base = OTHER; note = misc_feature - Phosphorothioate bond
  modified_base 20..21: mod_base = OTHER; note = misc_feature - Phosphorothioate bond
  source 1..21: mol_type = other RNA
caggactcggctcacacatgc

SEQ ID NO: 208 - RNA, 21 nt, synthetic construct
  misc_feature 1..21: note = Description of Artificial Sequence: Synthetic oligonucleotide
  modified_base 1..2: mod_base = OTHER; note = misc_feature - Phosphorothioate bond
  modified_base 20..21: mod_base = OTHER; note = misc_feature - Phosphorothioate bond
  source 1..21: mol_type = other RNA
agacgacagctggatcacacg

SEQ ID NO: 209 - DNA, 26 bp, Homo sapiens
  source 1..26: mol_type = unassigned DNA
acctgggattctctacgaaattcagt

SEQ ID NO: 210 - DNA, 26 bp, Homo sapiens
  source 1..26: mol_type = unassigned DNA
ccttgattgaggtatagttcttgtct

SEQ ID NO: 211 - DNA, 26 bp, Homo sapiens
  source 1..26: mol_type = unassigned DNA
tggtgcttagtaaactcttggttcca

SEQ ID NO: 212 - DNA, 26 bp, Homo sapiens
  source 1..26: mol_type = unassigned DNA
ctgcaggagtaaggacaggaaggtgc

(19) United States
(12) Patent Application Publication
(10) Pub. No.: US 2025/0154598 A1
(43) Pub. Date: May 15, 2025

(54) NON-CODING RNA FOR DETECTION OF CANCER

(71) Applicant: The Regents of the University of California, Oakland, CA (US)

(72) Inventor: Hani Goodarzi, San Francisco, CA (US)

(73) Assignee: The Regents of the University of California, Oakland, CA (US)

(21) Appl. No.: 18/752,640

(22) Filed: Jun. 24, 2024

Related U.S. Application Data
(63) Continuation of application No. 18/489,807, filed on Oct. 18, 2023, now abandoned.
Application No. 18/489,807 is a continuation of application No. 16/763,540, filed on May 12, 2020, now abandoned.
(60) Provisional application No. 62/584,899, filed on Nov. 12, 2017.

(51) Int. Cl. C12Q 1/6886

(52) U.S. Cl. CPC C12Q 1/6886; C12Q 2600/158; C12Q 2600/178

(57) ABSTRACT

The present disclosure relates generally to detection on non-coding RNA molecules in a sample or diagnosis of subject based upon detection or quantification of non-coding nucleic acid sequences in a sample, specifically to identify and use of molecular biomarkers for cancer including breast cancer.

### CROSS-REFERENCE TO RELATED APPLICATIONS

This application is a continuation of U.S. application Ser. No. 18/489,807, which was filed Oct. 18, 2023, and was a continuation of U.S. application Ser. No. 16/763,540, which was a 35 U.S.C. § 371 national stage application of PCT/US2018/060113, which was filed Nov. 9, 2018, and claimed the benefit of U.S. Provisional Application No. 62/584,899, which was filed Nov. 12, 2017, all of which are incorporated herein by reference in its entirety.

### SUBMISSION OF SEQUENCE LISTING

The Sequence Listing associated with this application is filed in electronic format via Patent Center and is hereby incorporated by reference into the specification in its entirety. The name of the text file containing the Sequence Listing is “UCAL-007-US-CON3.xml,” created on Jun. 21, 2024, and having a size of 200,024 bytes.

### TECHNOLOGY FIELD

The present disclosure relates generally to detection of non-coding RNAs molecules in a sample or diagnosis of subject based upon detection or quantification of non-coding nucleic acid sequences in a sample, specifically to identify and use of molecular biomarkers for cancer including breast cancer.

### BACKGROUND

The widespread reprogramming of the gene expression landscape is a hallmark of cancer development. Thus, the systematic identification of regulatory pathways that drive pathologic gene expression patterns is a crucial step towards understanding and treating cancer. Over the years, a multitude of regulatory mechanisms have been implicated in oncogenic expression of genes involved in cancer cell differentiation, survival, invasion, and spread. While numerous studies have focused on the transcriptional pathways that underlie oncogenesis, post-transcriptional regulatory pathways have also emerged as major regulators of this process. For example, microRNAs (small non-coding RNAs), a subclass of small RNAs that function in gene silencing, were among the first characterized post-transcriptional regulators of breast cancer progression (1). RNA-binding proteins (RBPs) are also critical posttranscriptional regulators of gene expression, and several specific RBPs have been shown to affect oncogenesis and cancer progression (e.g. 2-5). Recently, it was demonstrated that tRNAs (6) and tRNA fragments (7), which are other classes of small non-coding RNAs, play a fundamental role in breast cancer progression.

Despite the diverse repertoire of regulatory mechanisms involved in cancers, a shared characteristic among them is that they co-opt and dys-regulate existing pathways within the cell. In other words, cancer cells adopt myriad strategies, such as somatic mutations (e.g. KRAS, 8), gene fusions (e.g. BCR-ABL, 9), epigenetic modifications (e.g. promoter hypermethylation, 10), and regulatory mechanisms disruptions (NFkB transcription factors, 10) to over-activate oncogenic and to down-regulate tumor suppressive pathways (11, 12). While these strategies rely on the pathologic modulation of regulatory programs that are already in place, there is an often-overlooked possibility that cancer cells may be capable of evolving or engineering specialized regulatory pathways that drive tumorigenesis.

### SUMMARY

The described invention provides novel small non-coding RNAs that serve as biomarkers which are indicative of cancer such as breast cancer, and which may be used to accurately diagnose breast cancer in a subject. In some embodiments, the methods comprise detection of extracellular, circulating small RNAs in a suitable sample. In some embodiments, the sample is a human serum sample. In some embodiments, the sample is a fractionated human serum sample comprising exosomes that comprise small non-coding mRNA.

The invention also relates to detecting the presence of non-coding RNAs in a blood or blood serum sample. In some embodiments, the disclosure relates to a method for detecting a hyperproliferative cell in a subject comprising detecting the absence, presence or quantity of non-coding nucleic acid in a serum or plasma sample. In some embodiments, the methods comprise isolating total RNA from the sample and detecting the presence of non-coding mRNA sequences and correlating the quantity of non-coding mRNA to the likelihood of whether the subject comprises one or a plurality of hyperproliferative cells. In some embodiments, the methods comprise isolating total RNA from the sample and detecting the presence of non-coding mRNA sequences and correlating the quantity of non-coding mRNA to the likelihood of whether the subject comprises one or a plurality of cancer cells. In some embodiments, the methods comprise isolating total RNA from the sample and detecting the presence of non-coding mRNA sequences and correlating the quantity of non-coding mRNA to the likelihood of whether the subject comprises one or a plurality of solid tumor cells. In some embodiments, the methods comprise isolating total RNA from the sample and detecting the presence of non-coding mRNA sequences and correlating the quantity of non-coding mRNA to the likelihood of whether the subject comprises one or a plurality of breast cancer cells.

In some embodiments, methods are described herein for determining a diagnosis comprising determining the presence of one or a combination of non-coding nucleic acids from a sample derived from a subject's plasma or serum sample according to the methods previously described and providing a diagnosis based on the presence of said one or combination of non-coding nucleic acids. In some embodiments, the diagnosis determined is cancer such as breast cancer.

In some embodiments, a computer implemented method is used for determining the presence or absence of one or a combination of non-coding nucleic acids comprising practicing the methods previously described, comprising quantitating the abundance of one or a combination of non-coding nucleic acids of reference from one or a plurality of samples comprising one or a combination of non-coding nucleic acids, computationally determining the normalized amount of one or a combination of non-coding nucleic acids in the one or plurality of samples, and determining the presence or absence of one or a combination of non-coding nucleic acids based on said normalized amount. In some embodiments, quantitation comprising sequencing the sample of total RNA isolated from a sample in question. In some embodiments, computational analysis is performed on sequence data derived from a whole blood or serum sample. In some embodiments, the results of the previously described computer implemented methods are output wherein said output could be a diagnosis, for example a diagnosis of a hyperproliferative disorder, such as breast cancer. For other embodiments, additional sample related information can be output, such as information with regards to presence or absence of known tumor antigens in a sample. Outputting can be by a variety of means as described herein, for example results can be output visually on, for example a computer monitor and the like, or output can be hardcopy, such as a printed paper report and the like.

### BRIEF DESCRIPTION OF THE DRAWINGS

FIG. 1A-FIG. 1C show the discovery, annotation, and validation of cancer-specific orphan non-coding RNAs in breast cancer. FIG. 1A is a heatmap representing the relative abundance of 437 small non-coding RNAs that are significantly expressed in breast cancer lines but not normal HUMECs. HUMECs were processed in triplicate, whereas all other cell lines were assayed in duplicate. FIG. 1B is a heatmap that shows that of the 437 small RNAs identified in (FIG. 1A), 201 were significantly expressed in breast tumor biopsy small RNA gene expression profiles collected as part of the Cancer Genome Atlas (TCGA-BRCA), and these 201 were also largely absent from the adjacent normal tissue collected from the ˜200 individuals in this dataset. FIG. 1C is a heatmap that shows that these 201 cancer-specific small RNAs were classified as orphan non-coding RNAs or oncRNAs and were independently validated in a third dataset comparing small RNA profiles from four normal epithelial samples and 10 patient-derived xenograft models.

FIG. 2A and FIG. 2B show that orphan small RNAs are relatively abundant in cancer cells and are largely undetected in normal tissue. In FIG. 2A, in order to identify cancer-specific small RNAs, those RNAs that are largely absent in normal cells/tissue but are generally expressed in cancer cells were searched. Such RNA species were identified from two independent sources: (i) profiling of breast cancer cell lines in comparison to HUMECs, and (ii) The Cancer Genome Atlas dataset (TCGA-BRCA) with ˜200 normal tissue biopsies and ˜1000 tumor samples. These two independent sets were then overlapped to identify orphan non-coding RNAs (oncRNAs). As shown in FIG. 2A, a strong overlap was seen between these two analyses (hypergeometric P˜0). In FIG. 2B, the total abundance of the 201 oncRNAs were calculated across four normal epithelial samples and 10 PDX models. As shown in FIG. 2B, total oncRNA expression can perfectly predict whether samples are cancerous or normal (both AUC and AUPRC equals 1.0).

FIG. 3A-FIG. 3F show that the oncRNA T3p is strongly associated with breast cancer progression. FIG. 3A is a volcano plot comparing the expression of oncRNAs in poorly metastatic breast cancer cells relative to their highly metastatic derivatives. Highlighted is the oncRNA T3p, which is significantly upregulated in highly metastatic cells. FIG. 3B is a schematic that shows that T3p maps to the 3′ end (CR7 domain) of TERC, the RNA component of telomerase. FIG. 3C shows that expression of T3p (cpm) in breast tumor biopsies and their matched normal tissue in the TCGA-BRCA dataset. Paired Wilcoxon test was used to calculate the associated p-value. FIG. 3D shows T3p expression across the TCGA-BRCA dataset. FIG. 3E shows survival analysis in the TCGA-BRCA dataset for patients stratified based on the expression of T3p in their tumors. Top and bottom third of samples were included in this analysis (Log-rank test). FIG. 3F shows expression of T3p across normal, stage I, and stage II or III samples in the TCGA-BRCA dataset (*, P<0.05; ***, P<0.001; using Mann-Whitney test).

FIG. 4A-D shows that the oncRNA T3p is associated with aggressive breast cancers. FIG. 4A shows a tabulation of the number of samples in the TCGA-BRCA dataset based on the sample type (normal vs. breast cancer). Also included is the Fisher exact test χ² test associated with each contingency table. FIG. 4B shows a stratification of patients in the TCGA-BRCA dataset based on whether T3p was detected in their tumor biopsies or not. Mere detection of T3p in biopsies is associated with poor survival in breast cancer (P calculated based on a log-rank test). FIG. 4C shows a comparison of T3p expression levels in TCGA-BRCA samples divided based on ER, PR, or HER2 status. FIG. 4D shows T3p is significantly expressed in breast cancer PDX models (***, P<0.001; Mann-Whitney test).

FIG. 5 shows T3p promotes metastatic progression. Gene expression changes induced by an anti-T3p LNA is largely comparable regardless which control is used: (i) an scrambled LNA, or (ii) an anti-TERC (but not T3p) LNA. Included is the Pearson correlation coefficient and the associated p-value.

FIG. 6A-FIG. 6C shows T3p as a gene expression regulator and a driver of metastatic progression. FIG. 6A shows a comparison of gene expression changes induced by anti-T3p LNAs in MDA-LM2 cells versus T3p mimetics in MDA-MB-231 cells. Reported is the associated Pearson correlation (P˜0). FIG. 6B shows a bioluminescence imaging plot of lung metastasis by MDA-LM2 cells transfected with anti-T3p LNAs (LNA-T3p) or scrambled LNAs (LNA-Scr); n=4 or 5 in each cohort. Statistical significance was measured using two-way ANOVA. The area under the curve was also calculated for each mouse (change in normalized lung photon flux times days elapsed). Error bars indicate s.e.m. **, P<0.01 by a one-tailed Mann-Whitney test. FIG. 6C graphically shows the number of visible metastatic nodules that were counted in three mice from each cohort. The panel on the right shows hematoxylin and eosin stain (H&E) stained representative lung sections from each cohort along with the median counts. Error bars indicate s.e.m. *, P<0.05 by a one-tailed Mann-Whitney test.

FIG. 7A-FIG. 7C shows systematic profiling of oncRNAs in the exosomal compartment. FIG. 7A shows a large fraction of oncRNAs were detected in exosomal small RNA data collected from MDA-MB-231 cells but not normal HUVEC cells. FIG. 7B shows small RNA profiling of exososmal RNA collected from breast cancer cell lines and normal HUMECs. Shown is a heatmap showing the detection of oncRNAs among the extracellular population. FIG. 7C shows the detection of oncRNAs in serum samples collected from breast cancer patients with stage II and III disease. As a point of reference, data from 11 healthy individuals from an independent study is used as a reference.

FIG. 8A and FIG. 8B show T3p can be detected in the exosomal and circulating compartments. FIG. 8A shows the results of validation of T3p upregulation in highly metastatic MDA-LM2 cells relative to poorly metastatic parental cells in a previously published small RNA-seq data (7) and quantitative RT-PCR (n=6 in each sample, **, P<0.01; using a two-tailed Mann-Whitney test. FIG. 8B shows T3p can be detected at high levels in the absolute majority of sera collected from patients but is present at very low levels (or undetected) in serum samples collected from healthy individuals.

FIG. 9A through FIG. 9C show that the oncRNA T3p is associated with aggressive breast cancers. FIG. 9A shows normalized T3p expression from small RNA sequencing of the indicated cell lines on the x-axis. All cancer lines were prepared and processed in biological duplicates and HMECs in biological triplicate. Cell lines shape-coded by sub-type: HMEC (circles on left hand side of panel), triple negative breast cancer (TNBC; squares, triangle and diamonds in the middle of the panel), HER2 positive (circles and squares to the right-hand side of the panel), and luminal (triangles to the right hand side of the panel). FIG. 9B depicts a comparison of T3p expression levels in TCGA-BRCA samples divided based on ER, PR, or HER2 status (n=1033, 1030, and 715, respectively). The mean±s.d. are shown for each cohort.

FIG. 9C depict the relative T3p expression measured by qRT-PCR in two poorly and two highly metastatic breast cancer PDX models.

FIG. 10A through FIG. 10F show that T3p can be detected in the extracellular and circulating compartments. FIG. 10A depicts that T3p was present in the sequenced small RNA isolated from extracellular vesicles (EVs) from 7/8 breast cancer cell lines, and not present in HMEC EVs. Samples were processed and prepared in biological replicates and combined prior to calculation of counts-per-million. Cell lines shape-coded by sub-type: HMEC (the first column on the X-axis beginning from the left), triple negative breast cancer (TNBC; the next four columns to the right of HMEC), HER2 positive (the next two columns on the right of the HER2 set of samples), and luminal (the last two columns on the rightmost side of the graph). FIG. 10B shows Pearson correlation coefficients between oncRNA expression levels in total intracellular (IC) and extracellular (CM) compartments, as well as IC and extracellular vesicle (EV) compartments. n=2 biologically independent experiments per cell line. FIG. 10C shows 10 bootstrap based receiver operating characteristic (ROC) curves depicting the classification performance of a gradient boosted classifier trained on oncRNA expression levels in the TCGA-BRCA dataset and tested on serum samples from healthy volunteers or breast cancer patients (GSE49035). FIG. 10D shows T3p can be detected at high levels in the absolute majority of sera collected from patients but is present at very low levels (or undetected) in serum samples collected from healthy individuals. The panel on the right shows T3p levels in sera collected from individual breast cancer patients. n=40 biologically independent samples. Shown are mean±s.e.m; P was calculated using a two-tailed Mann Whitney test. FIG. 10E depicts that T3p can be detected at high levels in the absolute majority of sera collected from patients but is present at very low levels (or undetected) in serum samples collected from healthy individuals. The panel on the right shows T3p levels in sera collected from individual breast cancer patients. n=40 biologically independent samples. Shown are mean±s.e.m; P was calculated using a two-tailed Mann Whitney test. Bootstrapped ROC curves (10 times) were generated for a gradient boosted classifier trained on miRNAs expression in the TCGA-BRCA dataset, and tested on serum samples from healthy volunteers or breast cancer patients (data not shown).

### DETAILED DESCRIPTION

The disclosure provides novel small non-coding RNAs that serve as biomarkers which are indicative of breast cancer, and which may be used to accurately diagnose or grade breast cancer in a subject. In some embodiments, the methods entail detection of extracellular, circulating small RNAs in a suitable sample.

### Definitions

Prior to setting forth the invention in detail, definitions of certain terms to be used herein are provided.

Unless defined otherwise, technical and scientific terms used herein have the same meaning as commonly understood by one of ordinary skill in the art to which this invention belongs. For example, Singleton et al., Dictionary of Microbiology and Molecular Biology 2nd ed., J. Wiley & Sons (New York, NY 1994), provide one skilled in the art with a general guide to many of the terms used in the present application. Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” or “approximately” as used herein is meant to refer to within 5%, within 4%, within 3%, within 2%, within 1%, of a given value or range.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site. As used herein, the term encompasses intact polyclonal antibodies, intact monoclonal antibodies, single chain antibodies, antibody fragments (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological binding activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site. The term “variable region” of an antibody refers to the variable region of an antibody light chain, or the variable region of an antibody heavy chain, either alone or in combination. The variable region of a heavy chain or a light chain generally consists of four framework regions (FR) connected by three complementarity determining regions (CDRs), also known as “hypervariable regions”. The CDRs in each chain are held together in close proximity by the framework regions and contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda, MD), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “biomarker” as used herein refers to a biological molecule present in an individual at varying concentrations useful in predicting the cancer status of an individual. A biomarker may include but is not limited to, nucleic acids, proteins and variants and fragments thereof. A biomarker may be DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest.

The term “bodily fluid” as used herein refers to a bodily fluid comprising non-coding RNA (ncRNA) including blood (or a fraction of blood such as plasma or serum), lymph, mucus, tears, saliva, sputum, urine, semen, stool, CSF (cerebrospinal fluid), breast milk, and, ascities fluid. In some embodiments, the bodily fluid is urine. In some embodiments, the bodily fluid is fractionated serum comprising exosomes.

The terms “cancer” and “cancerous” as used herein refer to or describe the physiological condition in mammals in which a population of cells are characterized by unregulated cell growth. In some embodiments, the cancer is a breast cancer.

The term “correlate” or “correlating” as used herein refers to a statistical association between instances of two events, where events may include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases. The present invention provides small non-coding RNAs, the levels of which are correlated with a particular outcome measure, such as between the level of a small non-coding RNA and the likelihood of developing breast cancer. For example, the increased level of a small non-coding RNA may be negatively correlated with a likelihood of good clinical outcome for the patient. In this case, for example, the patient may have a decreased likelihood of long-term survival without recurrence of the cancer and/or a positive response to a chemotherapy, and the like. Such a negative correlation indicates that the patient likely has a poor prognosis or will respond poorly to a chemotherapy, and this may be demonstrated statistically in various ways, e.g., by a high hazard ratio.

The term “high stringency” as used herein refers to conditions that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ during hybridization 50% formamide in 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 50% formamide, followed by a wash consisting of 0.1×SSC containing EDTA at 55° C.

The term “hyperproliferative disorder” refers to a disease or disorder characterized by abnormal proliferation, abnormal growth, abnormal senescence, abnormal quiescence, or abnormal removal of cells in an organism, and includes all forms of hyperplasias, neoplasias, and cancer. In some embodiments, the hyperproliferative disease is a cancer derived from the gastrointestinal tract or urinary system. In some embodiments, a hyperproliferative disease is a cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus. In some embodiments, the term hyperproliferative disease is a cancer chosen from: lung cancer, bone cancer, CMML, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

The terms “identical” or “percent identity” or “homology” in the context of two or more nucleic acids, as used herein, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids of the invention are substantially identical, meaning they have at least about 70%, at least about 75%, at least about 80%, at least about 85%, at least about 90%, and in some embodiments at least about 95%, 96%, 97%, 98%, 99% nucleotide or amino acid residue sequence identity, when compared and aligned for maximum correspondence, as measured using a sequence comparison algorithm or by visual inspection. In some embodiments, identity exists over a region of the sequences that is at least about 10, at least about 20, at least about 40-60 nucleotides, at least about 60-80 nucleotides or any integral value therebetween. In some embodiments, identity exists over a longer region than 60-80 nucleotides, such as at least about 80-100 nucleotides, and in some embodiments the sequences are substantially identical over the full length of the sequences being compared.

The term “level” as used herein refers to qualitative or quantitative determination of the number of copies of a non-coding RNA transcript. An RNA transcript exhibits an “increased level” when the level of the RNA transcript is higher in a first sample, such as in a clinically relevant subpopulation of patients (e.g., patients who have cancer), than in a second sample, such as in a related subpopulation (e.g., patients who do not have cancer). In the context of an analysis of a level of an RNA transcript in a tumor sample obtained from an individual patient, an RNA transcript exhibits “increased level” when the level of the RNA transcript in the subject trends toward, or more closely approximates, the level characteristic of a clinically relevant subpopulation of patients.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates (e.g., via the bloodstream or lymph) from the primary site of disease to secondary sites.

The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “normalized” as used herein with regard to non-coding RNA transcript, refers to the level of the RNA transcript, relative to the mean levels of transcript of a set of reference RNA transcripts. The reference RNA transcripts are based on their minimal variation across patients, tissues, or treatments. Alternatively, the non-coding RNA transcript may be normalized to the totality of tested RNA transcripts, or a subset of such tested RNA transcripts.

A “patient response” may be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the cancer; (8) increase in the length of survival following treatment; and/or (9) decreased mortality at a given point of time following treatment.

The terms “polynucleotide” and “nucleic acid” and “nucleic acid molecule” are used interchangeably herein and refer to polymers of nucleotides of any length, and include DNA and RNA. The polynucleotides can be deoxyribonucleotides, ribonucleotides, modified nucleotides or bases, and/or their analogs, or any substrate that can be incorporated into a polymer by DNA or RNA polymerase.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or fusion proteins, in certain embodiments, the polypeptides can occur as single chains or associated chains (e.g., dimers).

The term “prognosis” as used herein refers to the prediction of the likelihood of cancer-attributable death or progression, including recurrence, metastatic spread, and drug resistance, of neoplastic disease, such as breast cancer.

The term “reference” RNA transcript as used herein refers to an RNA transcript whose level can be used to compare the level of an RNA transcript in a test sample. In an embodiment of the invention, reference RNA transcripts include housekeeping genes, such as beta-globin, alcohol dehydrogenase, or any other RNA transcript, the level or expression of which does not vary depending on the disease status of the cell containing the RNA transcript. In another embodiment, all of the assayed RNA transcripts, or a subset thereof, may serve as reference RNA transcripts.

The term “small non-coding RNA” (ncRNA) as used herein, refers to RNA that is not translated into protein and includes transfer RNA (tRNA), ribosomal RNA (rRNA), snoRNAs, microRNA (miRNA), siRNAs, small nuclear (snRNA), Y RNA, vault RNA, antisense RNA, tiRNA (transcription initiation RNA), TSSa-RNA (transcriptional start-site associated RNA) and piwiRNA (piRNA). Small ncRNA have a length of less than 200 nucleotides. Preferably, a small ncRNA as used herein is between 50 and 100 nucleotides. A ncRNA may be of endogenous origin (e.g., a human small non-coding RNA) or exogenous origin (e.g., virus, bacteria, parasite). “Canonical” ncRNA refers to the sequence of the RNA as predicted from the genome sequence and is the most abundant sequence identified for a particular RNA. “Trimmed” ncRNA refers to an ncRNA in which exonuclease-mediated nucleotide trimming has removed one or more nucleotides at the 5′ and/or 3′ end of the molecule. “Extended ncRNA” refers to an small non-coding RNA that is longer than the canonical small non-coding RNA sequence and is a term recognized in the art. The nucleotides making up the extension correspond to nucleotides of the precursor sequence and are therefore encoded by the genome in contrast to non-templated nucleotide addition. In some embodiments, any of the methods disclosed herein comprise detecting any one or combination of RNAs disclosed above.

The term “subject” as used herein refers to any animal (e.g., a mammal), including, but not limited to, humans, non-human primates, canines, felines, rodents, and the like. Preferably, the subject is a human subject. The terms “subject,” “individual,” and “patient” are used interchangeably herein. The terms “subject,” “individual,” and “patient” thus encompass individuals having cancer (e.g., breast cancer), including those who have undergone or are candidates for resection (surgery) to remove cancerous tissue.

The term “therapeutically effective amount” means a quantity sufficient to achieve a desired therapeutic effect, for example, an amount which results in the prevention or amelioration of or a decrease in the symptoms associated with a disease that is being treated, e.g., disorders associated with cancer growth or a hyperproliferative disorder. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation. Typically, an effective amount of the compounds of the present invention, sufficient for achieving a therapeutic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds disclosed herein can also be administered in combination with each other, or with one or more additional therapeutic compounds.

The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention. n vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base. Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which cannot be considered pharmaceutically acceptable, can be useful in the preparation of salts useful as intermediates in obtaining a chemical compound of the invention and its pharmaceutically acceptable acid addition salt. Metal salts of a chemical compound of the invention include alkali metal salts, such as the sodium salt of a chemical compound of the invention containing a carboxy group. Mixtures of isomers obtainable according to the invention can be separated in a manner known per se into the individual isomers; diastereoisomers can be separated, for example, by partitioning between polyphasic solvent mixtures, recrystallization and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

The terms “treating” or “treatment” or “treat” as used herein refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already diagnosed with the disorder; those prone to have the disorder; and those in whom the disorder is to be prevented. In some embodiments, a subject is successfully “treated” according to the methods of the present invention if the patient shows one or more of the following: a reduction in the number of and/or complete absence of cancer cells; a reduction in the tumor size; an inhibition of tumor growth; inhibition of and/or an absence of cancer cell infiltration into peripheral organs including the spread of cancer cells into soft tissue and bone; inhibition of and/or an absence of tumor or cancer cell metastasis; inhibition and/or an absence of cancer growth; relief of one or more symptoms associated with the specific cancer; reduced morbidity and mortality; improvement in quality of life; reduction in tumorigenicity; reduction in the number or frequency of cancer stem cells; or some combination of such effects.

The term “tumor” as used herein, refers to all neoplastic cell growth and proliferation, whether malignant or benign, and all pre-cancerous and cancerous cells and tissues.

The term “T3p” refers to the last 45 nucleotides of non-coding RNA (in a 5′ to 3′ orientation) encoded or generated by the human TERC nucleotide seqeunce. The sequence may be found in PCT serial No. PCT/US2008/055709 and associated sequence listing, the contents of which are hereby incorporated by reference in their entirety.

The term “tumor sample” as used herein refers to a sample comprising tumor material obtained from a cancer patient. The term encompasses tumor tissue samples, for example, tissue obtained by surgical resection and tissue obtained by biopsy, such as for example, a core biopsy or a fine needle biopsy. In a particular embodiment, the tumor sample is a fixed, wax-embedded tissue sample, such as a formalin-fixed, paraffin-embedded tissue sample. Additionally, the term “tumor sample” encompasses a sample comprising tumor cells obtained from sites other than the primary tumor, e.g., circulating tumor cells. The term also encompasses cells that are the progeny of the patient's tumor cells, e.g. cell culture samples derived from primary tumor cells or circulating tumor cells. The term further encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

### Small RNA Biomarkers of Cancer

The human genome encodes for a vast amount of small non-protein-coding RNA (ncRNAs) transcripts. Multiple ncRNA classes have been described including the highly abundant transfer RNAs (tRNAs), ribosomal RNAs (rRNAs), small nucleolar RNAs (snoRNAs), microRNAs (miRNAs), small interfering RNAs (siRNAs), small nuclear RNAs (snRNAs), and piwi-interacting RNAs (piRNAs) (Amaral et al., 2008; Martens-Uzunova et al., 2013). Small non-coding RNAs act as translational repressors by binding to target mRNAs at sites with adequate sequence complementary (Ameres et al., 2007), while the highly abundant cytoplasmic Y RNAs function in RNA quality control by affecting the subcellular location of Ro proteins (Sim et al., 2009). The repressive activity of mature small non-coding RNAs on mRNA translation is shared by other classes of ncRNAs, including siRNAs and endo-siRNAs, in addition to piRNAs that silence retrotransposons at defined subcellular locations (Chuma and Pillai, 2009). Small non-coding RNA activity relies on sufficient levels of abundance in the cytoplasm, and interaction with RNA-induced silencing complexes (RISC) localized at endosomal membranes (Gibbings et al., 2009; Lee et al., 2009a), whereas low abundant small non-coding RNAs have less impact on translational repression. As a consequence, subtle alterations in the levels of certain small non-coding RNA may already influence cellular processes, while strong perturbations can cause disease. Besides abundance, interactions with (RISC) proteins but also RNA partners and correct subcellular localization are interrelated factors that control small non-coding RNA physiology (Mullokandov et al., 2012; Wee et al., 2012).

Small RNAs can be secreted in cell-derived extracellular vesicles, such as exosomes. Both mRNA and small non-coding RNA species have been found contained in exosomes. As such, exosomes can provide a means for transfer and protection of RNA content from degradation in the environment, enabling a stable source for reliable detection of RNA biomarkers.

The disclosure relates to small non-coding RNA biomarkers found to be differentially present in biological samples derived from subjects having breast cancer, as compared with subjects who are “normal,” i.e., subjects who do not have breast cancer. A small non-coding RNA biomarker or set of small non-coding RNA biomarkers is differentially present between samples if the difference between the levels of expression of the small non-coding RNA biomarker or set of small non-coding RNA biomarkers in the samples is determined to be statistically significant. Common tests for statistical significance include, but are not limited to, t-test, ANOVA, Kniskal-Wallis, Wilcoxon, Mann-Whitney, and odds ratio. small non-coding RNA biomarkers, alone or in combination, can be used to provide a measure of the relative risk that a subject has or does not have cancer.

Small non-coding RNA biomarkers of breast cancer were discovered by small RNA sequencing of multiple breast cancer subtypes as well as human mammary epithelial cells, and identifying previously unknown small non-coding RNAs that are specifically expressed in breast cancer cells. 200 previously unknown small non-coding RNAs that are specifically expressed in breast cancer cells were identified in this manner (see Table 1). These small non-coding RNA biomarkers can now be used to determine the cancer status of a subject, for example, a subject whose breast cancer status, was previously unknown or who is suspected to be suffering from breast cancer. This may be accomplished by determining the level of one or more of the identified small non-coding RNAs, or combinations thereof, in a biological sample derived from the subject. A difference in the level of one or more of these small non-coding RNA biomarkers as compared to that in a biological sample derived from a normal subject is an indication that the subject has breast cancer.

A subject having a difference in the level of one or more small non-coding RNA biomarkers as compared to a normal subject may have breast cancer, including early-stage, moderate or mid-stage, or severe or late-stage breast cancer. In one embodiment, the level of one or more small non-coding RNA biomarkers may be used to diagnose breast cancer, in a subject having symptoms characteristic of early-stage cancer.

In one embodiment, the level of one or more small non-coding RNA biomarkers may be used to monitor the course of cancer progression, for example breast cancer progression, in a subject. The cancer status of a subject can change over time. For example, the cancer may worsen or improve over time. With such worsening or improvement, the level of one or more small non-coding RNA biomarkers may change in a statistically significant fashion, as detected in samples derived from the subject. For example, the level of one or more of a small non-coding RNA biomarker may increase over time with the development of breast cancer. Thus, the course of breast cancer, progression, in a subject can be monitored by determining the level of one or more small non-coding RNA biomarkers in a first sample derived from a subject, and determining the level of one or more small non-coding RNA biomarkers in a second sample derived from a subject, where the second sample is obtained after the first sample. The levels in the second sample relative to the levels in the first sample are indicative of disease progression. For example, an increase in the level of one or more of a small non-coding RNA biomarker from Table 1, Table 2 or Table 3, from the first sample to the second sample is indicative that the subject has developed breast cancer, or that the disease has worsened. Conversely, a decrease in the level of one or more of a small non-coding RNA biomarker from Table 1, Table 2 or Table 3 from the first sample to the second sample indicates that the disease has improved. In one embodiment, the one or more small non-coding RNA biomarkers are from Table 3, and combinations thereof.

Whether or not the level of a small non-coding RNA biomarker in a biological sample derived from a test subject is different from the level of the small non-coding RNA biomarker present in a normal subject may be ascertained by comparing the level of the small non-coding RNA biomarker in the sample from the test subject with a suitable control. The skilled person can select an appropriate control for the assay in question. For example, a suitable control may be a biological sample derived from a known subject, e.g., a subject known to be a normal subject that does not have cancer. If a suitable control is obtained from a normal subject, a statistically significant difference in the level of a small non-coding RNA biomarker in a test subject relative to the suitable control is indicative that the subject has breast cancer. In one embodiment, the difference in the level of a small non-coding RNA biomarker is an increase. A suitable control may also be a reference standard. A reference standard serves as a reference level for comparison, such that test samples can be compared to the reference standard in order to infer the breast cancer, status of a subject. A reference standard may be representative of the level of one or more small non-coding RNA biomarkers in a known subject, e.g., a subject known to be a normal subject, or a subject known to have breast cancer. Likewise, a reference standard may be representative of the level of one or more small non-coding RNA biomarkers in a population of known subjects, e.g., a population of subjects known to be normal subjects, or a population of subjects known to have breast cancer. The reference standard may be obtained, for example, by pooling samples from a plurality of individuals and determining the level of a small non-coding RNA biomarker in the pooled samples, to thereby produce a standard over an averaged population. Such a reference standard represents an average level of a small non-coding RNA biomarker among a population of individuals. A reference standard may also be obtained, for example, by averaging the level of a small non-coding RNA biomarker determined to be present in individual samples obtained from a plurality of individuals. Such a standard is also representative of an average level of a small non-coding RNA biomarker among a population of individuals. A reference standard may also be a collection of values each representing the level of a small non-coding RNA biomarker in a known subject in a population of individuals. In certain embodiments, test samples may be compared against such a collection of values in order to infer the breast cancer, status of a subject. In certain embodiments, the reference standard is an absolute value. In such embodiments, test samples may be compared against the absolute value in order to infer the breast cancer, status of a subject. In a one embodiment, a comparison between the level of one or more small non-coding RNA biomarkers in a sample relative to a suitable control is made by executing a software classification algorithm. In some embodiments, the increased expression of one or a combination non-coding RNAs in Table 1, 2 and/or 3 wherein the increased expression is about 10%, 20%, 30%, 40%, 50%, 60%, 70%, 80%, 90%, 95 percent or about 100% or more expression than the expression of the same non-coding RNA in a normal sample. In some embodiments, the increased expression of one or a combination non-coding RNAs in Table 1, 2 and/or 3 wherein the increased expression is about 2×, 3×, 4×, 5×, 6×, 7×, 8×, 9× or about 10× or more expression than the expression of the same one or combination of non-coding RNA in a normal sample. In some embodiments, one or a plurality of the non-coding RNA sequences or nucleic acid sequences with about 70%, 80%, 81%, 82%, 83%, 84, 85%, 86%, 87%, 88%, 89%, 90%, 91%, 92%, 93%, 94%, 95%, 96%, 97%, 98%, or about 99% homology to the nucleic acid sequences in Tables 1, 2, and/or 3. In some embodiments, the mere presence or expression of one or a plurality non-coding RNAs alone or in combination with expression of one or a plurality of the sequences in Tables 1, 2, or 3. In some embodiments, the mere presence or expression of one or a plurality non-coding RNAs homolgous to alone or in combination with expression of one or a plurality of the sequences with about 70%, 80%, 81%, 82%, 83%, 84, 85%, 86%, 87%, 88%, 89%, 90%, 91%, 92%, 93%, 94%, 95%, 96%, 97%, 98%, or about 99% homology to the nucleic acid sequences in Tables 1, 2, and/or 3. In some embodiments, these homologous sequences comprise one or a plurality of fragments of the nucleic acid sequences disclosed in Tables 1, 2, and/or 3.

The skilled person can readily envision additional suitable controls that may be appropriate depending on the assay in question. The aforementioned suitable controls are exemplary, and are not intended to be limiting.

Generally, an increase in the level of one or more of a small non-coding RNA biomarker from Table 1, Table 2 or Table 3 in a biological sample derived from a test subject relative to a suitable control representative of the level of one or more of a small non-coding RNA biomarker from Table 1, Table 2 or Table 3 in a normal subject will indicate that the test subject has breast cancer. In some instances where the levels of two or more small non-coding RNA biomarkers are determined in a test subject, there may be an increase in the level of one or more small non-coding RNA biomarkers, and no change or an increase in the level of one or more additional small non-coding RNA biomarkers, relative to a suitable control. In such instances, a difference in the level of one or more of the small non-coding RNA biomarkers relative to a suitable control representative of the level of the small non-coding RNA biomarkers in a normal subject indicates that the test subject has breast cancer. Determination of such a difference may be aided by the execution of a software classification algorithm, as described herein.

### Biological Samples

The expression level of one or more small non-coding RNA biomarkers may be determined in a biological sample derived from a subject. A sample derived from a subject is one that originates from a subject. Such a sample may be further processed after it is obtained from the subject. For example, RNA may be isolated from a sample. In this example, the RNA isolated from the sample is also a sample derived from a subject. A biological sample useful for determining the level of one or more small non-coding RNA biomarkers may be obtained from essentially any source, including cells, tissues, and fluids throughout the body.

In some embodiments, the biological sample used for determining the level of one or more small non-coding RNA biomarkers is a sample containing circulating small non-coding RNAs, e.g., extracellular small non-coding RNAs. Extracellular small non-coding RNAs freely circulate in a wide range of biological material, including bodily fluids, such as fluids from the circulatory system, e.g., a blood sample or a lymph sample, or from another bodily fluid such as urine or saliva. Accordingly, in some embodiments, the biological sample used for determining the level of one or more small non-coding RNA biomarkers is a bodily fluid, for example, blood, fractions thereof, serum, plasma, urine, saliva, tears, sweat, semen, vaginal secretions, lymph, bronchial secretions, CSF, whole blood, etc. In some embodiments, the sample is a sample that is obtained non-invasively. In some embodiments, the sample is a serum sample from a human.

In some embodiments, any of the methods disclosed herein comprise using a small volume sample. In some embodiments, the methods disclosed comprise isolating total RNA and/or amplifying non-coding RNA in a sample of no more than about 20 microliters of sample, 40 microliters of sample, 80 microliters of sample, 100 microliters of sample, 200 microliters of sample, 300 microliters of sample, 400 microliters of sample, 500 microliters of sample, 600 microliters of sample, 700 microliters of sample, 800 microliters of sample, 900 microliters of sample, 1 milliter of sample, 1.1 milliters of sample, 1.2 milliters of sample, 1.3 milliters of sample, 1.4 milliters of sample, 1.5 milliters of sample, 1.6 milliters of sample, 1.7 milliters of sample, 1.8 milliters of sample, 1.9 milliters of sample, 2.0 milliters of sample. In some embodiments, the sample size is from about 25 microliters to about 2 milliliters of liquid sample in the form of subject plasma, whole blood or serum.

In some embodiments, the methods disclosed comprise isolating total RNA and/or amplifying non-coding RNA in a sample of no more than about 20 microliters of serum, 40 microliters of serum, 80 microliters of serum, 100 microliters of serum, 200 microliters of serum, 300 microliters of serum, 400 microliters of serum, 500 microliters of serum, 600 microliters of serum, 700 microliters of serum, 800 microliters of serum, 900 microliters of serum, 1 milliter of serum, 1.1 milliters of serum, 1.2 milliters of serum, 1.3 milliters of serum, 1.4 milliters of serum, 1.5 milliters of serum, 1.6 milliters of serum, 1.7 milliters of serum, 1.8 milliters of serum, 1.9 milliters of serum, 2.0 milliters of serum.

Circulating small non-coding RNAs include small non-coding RNAs in cells, extracellular small non-coding RNAs in microvesicles, in exosomes and extracellular small non-coding RNAs that are not associated with cells or microvesicles (extracellular, non-vesicular small non-coding RNA). In some embodiments, the biological sample used for determining the level of one or more small non-coding RNA biomarkers (e.g., a sample containing circulating small non-coding RNA) may contain cells. In other embodiments, the biological sample may be free or substantially free of cells (e.g., a serum sample). In some embodiments, a sample containing circulating small non-coding RNAs, e.g., extracellular small non-coding RNAs, is a blood-derived sample. Exemplary blood-derived sample types include, e.g., a plasma sample, a serum sample, a blood sample, etc. In other embodiments, a sample containing circulating small non-coding RNAs is a lymph sample. Circulating small non-coding RNAs are also found in urine and saliva, and biological samples derived from these sources are likewise suitable for determining the level of one or more small non-coding RNA biomarkers.

In some embodiments, any of the methods of the disclosure comprise the step of isolating total RNA from a sample or cell or exosome or microvesicle. Methods of isolating RNA for expression analysis from blood, plasma and/or serum (see for example, Tsui N B et al. (2002) Clin. Chem. 48, 1647-53, incorporated by reference in its entirety herein) and from urine (see for example, Boom R et al. (1990) J Clin Microbiol. 28, 495-503, incorporated by reference in its entirety herein) have been described.

### Determining the Level of Small RNA Biomarkers in a Sample

The level of one or more small non-coding RNA biomarkers in a biological sample may be determined by any suitable method. Any reliable method for measuring the level or amount of small non-coding RNA in a sample may be used. Generally, small non-coding RNA can be detected and quantified from a sample (including fractions thereof), such as samples of isolated RNA by various methods known for mRNA, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString analysis, Northern Blot analysis, branched DNA (bDNA) signal amplification, in situ hybridization, etc.), and sequencing-based methods (e.g., next-generation sequencing methods, for example, using the Illumina or IonTorrent platforms). Other exemplary techniques include ribonuclease protection assay (RPA) and mass spectroscopy.

In some embodiments, RNA is converted to DNA (cDNA) prior to analysis. cDNA can be generated by reverse transcription of isolated small non-coding RNA using conventional techniques. In some embodiments, small non-coding RNA is amplified prior to measurement. In other embodiments, the level of small non-coding RNA is measured during the amplification process. In still other embodiments, the level of small non-coding RNA is not amplified prior to measurement. Some exemplary methods suitable for determining the level of small non-coding RNA in a sample are described in greater detail below. These methods are provided by way of illustration only, and it will be apparent to a skilled person that other suitable methods may likewise be used.

### A. Amplification-Based Methods

Many amplification-based methods exist for detecting the level of small non-coding RNA nucleic acid sequences, including, but not limited to, PCR, RT-PCR, qPCR, and rolling circle amplification. Other amplification-based techniques include, for example, ligase chain reaction, multiplex ligatable probe amplification, in vitro transcription (IVT), strand displacement amplification, transcription-mediated amplification, RNA (Eberwine) amplification, and other methods that are known to persons skilled in the art.

A typical PCR reaction includes multiple steps, or cycles, that selectively amplify target nucleic acid species: a denaturing step, in which a target nucleic acid is denatured; an annealing step, in which a set of PCR primers (i.e., forward and reverse primers) anneal to complementary DNA strands, and an elongation step, in which a thermostable DNA polymerase elongates the primers. By repeating these steps multiple times, a DNA fragment is amplified to produce an amplicon, corresponding to the target sequence. Typical PCR reactions include 20 or more cycles of denaturation, annealing, and elongation. In many cases, the annealing and elongation steps can be performed concurrently, in which case the cycle contains only two steps. A reverse transcription reaction (which produces a cDNA sequence having complementarity to a small non-coding RNA) may be performed prior to PCR amplification. Reverse transcription reactions include the use of, e.g., a RNA-based DNA polymerase (reverse transcriptase) and a primer.

Kits for quantitative real time PCR of small non-coding RNA are known, and are commercially available. Examples of suitable kits include, but are not limited to, the TaqMan miRNA Assay (Applied Biosystems) and the mirVana. qRT-PCR miRNA detection kit (Ambion). The small non-coding RNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence.

In some instances, custom qRT-PCR assays can be developed for determination of small non-coding RNA levels. Custom qRT-PCR assays to measure small non-coding RNAs in a biological sample, e.g., a body fluid, can be developed using, for example, methods that involve an extended reverse transcription primer and locked nucleic acid modified PCR. Custom small non-coding RNA assays can be tested by running the assay on a dilution series of chemically synthesized small non-coding RNA corresponding to the target sequence. This permits determination of the limit of detection and linear range of quantitation of each assay. Furthermore, when used as a standard curve, these data permit an estimate of the absolute abundance of small non-coding RNAs measured in biological samples.

Amplification curves may optionally be checked to verify that Ct values are assessed in the linear range of each amplification plot. Typically, the linear range spans several orders of magnitude. For each candidate small non-coding RNA assayed, a chemically synthesized version of the small non-coding RNA can be obtained and analyzed in a dilution series to determine the limit of sensitivity of the assay, and the linear range of quantitation. Relative expression levels may be determined, for example, as described by Livak et al., Methods (2001) December; 25(4):402-8.

In some embodiments, two or more small non-coding RNAs are amplified in a single reaction volume. For example, multiplex q-PCR, such as qRT-PCR, enables simultaneous amplification and quantification of at least two small non-coding RNAs of interest in one reaction volume by using more than one pair of primers and/or more than one probe. The primer pairs comprise at least one amplification primer that specifically binds each small non-coding RNA, and the probes are labeled such that they are distinguishable from one another, thus allowing simultaneous quantification of multiple small non-coding RNAs.

Rolling circle amplification is a DNA-polymerase driven reaction that can replicate circularized oligonucleotide probes with either linear or geometric kinetics under isothermal conditions (see, for example, Lizardi et al., Nat. Gen. (1998) 19(3):225-232; Gusev et al., Am. J. Pathol. (2001) 159(1):63-69; Nallur et al., Nucleic Acids Res. (2001) 29(23):E118). In the presence of two primers, one hybridizing to the (+) strand of DNA, and the other hybridizing to the (−) strand, a complex pattern of strand displacement results in the generation of over 10\hat{ }9 copies of each DNA molecule in 90 minutes or less. Tandemly linked copies of a closed circle DNA molecule may be formed by using a single primer. The process can also be performed using a matrix-associated DNA. The template used for rolling circle amplification may be reverse transcribed. This method can be used as a highly sensitive indicator of small non-coding RNA sequence and expression level at very low small non-coding RNA concentrations (see, for example, Cheng et al., Angew Chem. Int. Ed. Engl. (2009) 48(18):3268-72; Neubacher et al., Chembiochem. (2009) 10(8):1289-91).

### B. Hybridization-Based Methods

Small non-coding RNA may be detected using hybridization-based methods, including but not limited to hybridization arrays (e.g., microarrays), NanoString analysis, Northern Blot analysis, branched DNA (bDNA) signal amplification, and in situ hybridization.

Microarrays can be used to measure the expression levels of large numbers of small non-coding RNAs simultaneously. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays. Also useful are microfluidic TaqMan Low-Density Arrays, which are based on an array of microfluidic qRT-PCR reactions, as well as related microfluidic qRT-PCR based methods.

Axon B-4000 scanner and Gene-Pix Pro 4.0 software or other suitable software can be used to scan images. Non-positive spots after background subtraction, and outliers detected by the ESD procedure, are removed. The resulting signal intensity values are normalized to per-chip median values and then used to obtain geometric means and standard errors for each small non-coding RNA. Each signal can be transformed to log base 2, and a one-sample t test can be conducted. Independent hybridizations for each sample can be performed on chips with each small non-coding RNA spotted multiple times to increase the robustness of the data.

Microarrays can be used for the expression profiling of small non-coding RNAs in diseases. For example, RNA can be extracted from a sample and, optionally, the small non-coding RNAs are size-selected from total RNA. Oligonucleotide linkers can be attached to the 5′ and 3′ ends of the small non-coding RNAs and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding small non-coding RNA capture probe sequence on the array will hybridize, via base pairing, to the spot at which the, capture probes are affixed. The spot will then fluoresce when excited using a microarray laser scanner.

The fluorescence intensity of each spot is then evaluated in terms of the number of copies of a particular small non-coding RNA, using a number of positive and negative controls and array data normalization methods, which will result in assessment of the level of expression of a particular small non-coding RNA.

Total RNA containing the small non-coding RNA extracted from a body fluid sample can also be used directly without size-selection of the small non-coding RNAs. For example, the RNA can be 3′ end labeled using T4 RNA ligase and a fluorophore-labeled short RNA linker. Fluorophore-labeled small non-coding RNAs complementary to the corresponding small non-coding RNA capture probe sequences on the array hybridize, via base pairing, to the spot at which the capture probes are affixed. The fluorescence intensity of each spot is then evaluated in terms of the number of copies of a particular small non-coding RNA, using a number of positive and negative controls and array data normalization methods, which will result in assessment of the level of expression of a particular small non-coding RNA.

Several types of microarrays can be employed including, but not limited to, spotted oligonucleotide microarrays, pre-fabricated oligonucleotide microarrays or spotted long oligonucleotide arrays.

Small non-coding RNAs can also be detected without amplification using the nCounter Analysis System (NanoString Technologies, Seattle, Wash.). This technology employs two nucleic acid-based probes that hybridize in solution (e.g., a reporter probe and a capture probe). After hybridization, excess probes are removed, and probe/target complexes are analyzed in accordance with the manufacturer's protocol. nCounter miRNA assay kits are available from NanoString Technologies, which are capable of distinguishing between highly similar small non-coding RNAs with great specificity.

Small non-coding RNAs can also be detected using branched DNA (bDNA) signal amplification (see, for example, Urdea, Nature Biotechnology (1994), 12:926-928). small non-coding RNA assays based on bDNA signal amplification are commercially available. One such assay is the QuantiGene® 2.0 miRNA Assay (Affymetrix, Santa Clara, Calif.).

Northern Blot and in situ hybridization may also be used to detect small non-coding RNAs. Suitable methods for performing Northern Blot and in situ hybridization are known in the art.

In some embodiments, biomarker expression is determined by an assay known to those of skill in the art, including but not limited to, multi-analyte profile test, enzyme-linked immunosorbent assay (ELISA), radioimmunoassay, Western blot assay, immunofluorescent assay, enzyme immunoassay, immunoprecipitation assay, chemiluminescent assay, immunohistochemical assay, dot blot assay, or slot blot assay. In some embodiments, wherein an antibody is used in the assay the antibody is detectably labeled. The antibody labels may include, but are not limited to, immunofluorescent label, chemiluminescent label, phosphorescent label, enzyme label, radiolabel, avidin/biotin, colloidal gold particles, colored particles, and magnetic particles. In some embodiments, biomarker expression is determined by an IHC assay.

In some embodiments, biomarker expression is determined using an agent that specifically binds the biomarker. Any molecular entity that displays specific binding to a biomarker can be employed to determine the level of that biomarker protein in a sample. Specific binding agents include, but are not limited to, antibodies, antibody fragments, antibody mimetics, and polynucleotides (e.g., aptamers). One of skill understands that the degree of specificity required is determined by the particular assay used to detect the biomarker protein. In some embodiments, the disclosure relates to a system comprising a solid support (such as an ELISA plate, gel, bead or column comprising an antibody, antibody fragment, antibody mimetic, and/or polynucleotides capable of binding to T3p or a salt thereof.

### C. Sequencing-Based Methods

Advanced sequencing methods can likewise be used as available. For example, small non-coding RNAs can be detected using Illumina. Next Generation Sequencing (e.g., Sequencing-By-Synthesis or TruSeq methods, using, for example, the HiSeq, HiScan, GenomeAnalyzer, or MiSeq systems (Illumina, Inc., San Diego, Calif.)). Small non-coding RNAs can also be detected using Ion Torrent Sequencing (Ion Torrent Systems, Inc., Gulliford, Conn.), or other suitable methods of semiconductor sequencing.

### D. Additional Small Non-Coding RNA Detection Tools

Mass spectroscopy can be used to quantify small non-coding RNA using RNase mapping. Isolated RNAs can be enzymatically digested with RNA endonucleases (RNases) having high specificity (e.g., RNase TI, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESI-MS. The presence of posttranscriptional modifications can be revealed by mass shifts from those expected based upon the RNA sequence. Ions of anomalous mass/charge values can then be isolated for tandem MS sequencing to locate the sequence placement of the posttranscriptionally modified nucleoside.

Matrix-assisted laser desorption/ionization mass spectrometry (MALDI-MS) has also been used as an analytical approach for obtaining information about posttranscriptionally modified nucleosides. MALDI-based approaches can be differentiated from ESI-based approaches by the separation step. In MALDI-MS, the mass spectrometer is used to separate the small non-coding RNA.

To analyze a limited quantity of intact small non-coding RNAs, a system of capillary LC coupled with nanoESI-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact small non-coding RNAs are eluted from the trap column and directly injected into a Cl 8 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for small non-coding RNA detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple small non-coding RNAs can be detected in a microarray format using a novel approach that combines a surface enzyme reaction with nanoparticle-amplified SPR imaging (SPRI). The surface reaction of poly(A) polymerase creates poly(A) tails on small non-coding RNAs hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for small non-coding RNA profiling at attamole levels.

### E. Detection of Amplified or Non-Amplified Small Non-Coding RNAs

In certain embodiments, labels, dyes, or labeled probes and/or primers are used to detect amplified or unamplified small non-coding RNAs. The skilled artisan will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where small non-coding RNA amplification is preferred.

A probe or primer may include standard (A, T or U, G and C) bases, or modified bases. Modified bases include, but are not limited to, the AEGIS bases (from Eragen Biosciences), which have been described, e.g., in U.S. Pat. Nos. 5,432,272, 5,965,364, and 6,001,983. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TAQMAN) probes (see U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,485,901 and 6,649,349), peptide nucleic acid (PNA) Molecular Beacons (see, e.g., U.S. Pat. Nos. 6,355,421 and 6,593,091), linear PNA beacons (see, e.g. U.S. Pat. No. 6,329,144), non-FRET probes (see, e.g., U.S. Pat. No. 6,150,097), Sunrise™/AmplifluorB™ probes (see, e.g., U.S. Pat. No. 6,548,250), stem-loop and duplex SCORPION probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), PNA light-up probes, antiprimer quench probes (Li et al., Clin. Chem. 53:624-633 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

Small non-coding RNAs can be detected by direct or indirect methods. In a direct detection method, one or more small non-coding RNAs are detected by a detectable label that is linked to a nucleic acid molecule. In such methods, the small non-coding RNAs may be labeled prior to binding to the probe. Therefore, binding is detected by screening for the labeled small non-coding RNA that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified small non-coding RNAs, are detected using FlexMAP Microspheres (Luminex) conjugated with probes to capture the desired nucleic acids. Some methods may involve detection with polynucleotide probes modified with fluorescent labels or branched DNA (bDNA) detection, for example.

In some embodiments, biomarker expression is determined using a PCR-based assay comprising specific primers and/or probes for each biomarker. As used herein, the term “probe” refers to any molecule that is capable of selectively binding a specifically intended target biomolecule. In some embodiments, herein, the term “probe” refers to any molecule that may bind or associate, indirectly or directly, covalently or non-covalently, to any of the substrates and/or reaction products and/or proteases disclosed herein and whose association or binding is detectable using the methods disclosed herein. In some embodiments, the probe is a fluorogenic probe, antibody or absorbance-based probes. If an absorbance-based probe, the chromophore pNA (para-nitroanaline) may be used as a probe for detection and/or quantification of a target nucleic acid sequence disclosed herein. In some embodiments the probe may be a nucleic acid sequence comprising a fluoregnic molecule or a substrate that when exposed to an enzyme becomes fluoregenic and the nucleci acid sequence is complementary to fragment of nucleic acid sequence comprising 70%, 80%, 81%, 82%, 83%, 84, 85%, 86%, 87%, 88%, 89%, 90%, 91%, 92%, 93%, 94%, 95%, 96%, 97%, 98%, 99% or about 100% sequence identity to any one or combination of nucleic acid sequences in Tables 1, 2, and/or 3.

The target molecule could be any one or combination of nucleic acid sequences identified in Tables 1, 2, and/or 3. In some embodiments, the target molecule is a nucleic acid sequence comprising 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any one or combination of nucleic acid sequences in Tables 1, 2, and/or 3. Probes can be synthesized by one of skill in the art using known techniques, or derived from biological preparations. Probes may include but are not limited to, RNA, DNA, proteins, peptides, aptamers, antibodies, and organic molecules. The term “primer” or “probe” encompasses oligonucleotides that have a specific sequence or oligonucleotides that have a specific sequence. In some embodiments, the target molecule is any amplified fragment of any one or combination of nucleic acid sequences identified in Tables 1, 2, and/or 3 and/or any one or combination of nucleic acid sequence comprising 70%, 80%, 81%, 82%, 83%, 84, 85%, 86%, 87%, 88%, 89%, 90%, 91%, 92%, 93%, 94%, 95%, 96%, 97%, 98%, or about 99% sequence identity to any one or combination of nucleic acid sequences in Tables 1, 2, and/or 3.

In other embodiments, nucleic acids are detected by indirect detection methods. For example, a biotinylated probe may be combined with a streptavidin-conjugated dye to detect the bound nucleic acid. The streptavidin molecule binds a biotin label on amplified small non-coding RNA, and the bound small non-coding RNA is detected by detecting the dye molecule attached to the streptavidin molecule. In one embodiment, the streptavidin-conjugated dye molecule comprises PHYCOLINK. Streptavidin R-Phycoerythrin (PROzyme). Other conjugated dye molecules are known to persons skilled in the art.

Labels include, but are not limited to: light-emitting, light-scattering, and light-absorbing compounds which generate or quench a detectable fluorescent, chemiluminescent, or bioluminescent signal (see, e.g., Kricka, L., Nonisotopic DNA Probe Techniques, Academic Press, San Diego (1992) and Garman A., Non-Radioactive Labeling, Academic Press (1997).). A dual labeled fluorescent probe that includes a reporter fluorophore and a quencher fluorophore is used in some embodiments. It will be appreciated that pairs of fluorophores are chosen that have distinct emission spectra so that they can be easily distinguished.

In certain embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In other embodiments, methods relying on hybridization and/or ligation to quantify small non-coding RNAs may be used, including oligonucleotide ligation (OLA) methods and methods that allow a distinguishable probe that hybridizes to the target nucleic acid sequence to be separated from an unbound probe. As an example, HARP-like probes, as disclosed in U.S. Publication No. 2006/0078894 may be used to measure the quantity of miRNAs. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified. A probe ligation reaction may also be used to quantify small non-coding RNAs. In a Multiplex Ligation-dependent Probe Amplification (MLPA) technique (Schouten et al., Nucleic Acids Research 30:e57 (2002)), pairs of probes which hybridize immediately adjacent to each other on the target nucleic acid are ligated to each other driven by the presence of the target nucleic acid. In some aspects, MLPA probes have flanking PCR primer binding sites. MLPA probes are specifically amplified when ligated, thus allowing for detection and quantification of small non-coding RNA biomarkers.

### Detecting a Level of Small RNA Biomarker

The small non-coding RNA biomarkers described herein can be used individually or in combination in diagnostic tests to assess the breast cancer status of a subject. Breast cancer status includes the presence or absence of breast cancer. Breast cancer status may also include monitoring the course of breast cancer, for example, monitoring disease progression. Based on the breast cancer status of a subject, additional procedures may be indicated, including, for example, additional diagnostic tests or therapeutic procedures.

The power of a diagnostic test to correctly predict disease status is commonly measured in terms of the accuracy of the assay, the sensitivity of the assay, the specificity of the assay, or the “Area Under a Curve” (AUC), for example, the area under a Receiver Operating Characteristic (ROC) curve. As used herein, accuracy is a measure of the fraction of misclassified samples. Accuracy may be calculated as the total number of correctly classified samples divided by the total number of samples, e.g., in a test population. Sensitivity is a measure of the “true positives” that are predicted by a test to be positive, and may be calculated as the number of correctly identified breast cancer samples divided by the total number of breast cancer samples. Specificity is a measure of the “true negatives” that are predicted by a test to be negative, and may be calculated as the number of correctly identified normal samples divided by the total number of normal samples. AUC is a measure of the area under a Receiver Operating Characteristic curve, which is a plot of sensitivity vs. the false positive rate (1-specificity). The greater the AUC, the more powerful the predictive value of the test. Other useful measures of the utility of a test include the “positive predictive value,” which is the percentage of actual positives who test as positives, and the “negative predictive value,” which is the percentage of actual negatives who test as negatives. In a preferred embodiment, the level of one or more small non-coding RNA biomarkers in samples derived from subjects having different breast cancer statuses show a statistically significant difference of at least p=0.05, e.g., p=0.05, p=0.01, p=0.005, p=0.001, etc. relative to normal subjects, as determined relative to a suitable control. In other preferred embodiments, diagnostic tests that use small non-coding RNA biomarkers described herein individually or in combination show an accuracy of at least about 75%, e.g., an accuracy of at least about 75%, about 80%, about 85%, about 90%, about 95%, about 97%, about 99% or about 100%. In other embodiments, diagnostic tests that use small non-coding RNA biomarkers described herein individually or in combination show a specificity of at least about 75%, e.g., a specificity of at least about 75%, about 80%, about 85%, about 90%, about 95%, about 97%, about 99% or about 100%. In other embodiments, diagnostic tests that use small non-coding RNA biomarkers described herein individually or in combination show a sensitivity of at least about 75%, e.g., a sensitivity of at least about 75%, about 80%, about 85%, about 90%, about 95%, about 97%, about 99% or about 100%. In other embodiments, diagnostic tests that use small non-coding RNA biomarkers described herein individually or in combination show a specificity and sensitivity of at least about 75% each, e.g., a specificity and sensitivity of at least about 75%, about 80%, about 85%, about 90%, about 95%, about 97%, about 99% or about 100% (for example, a specificity of at least about 80% and sensitivity of at least about 80%, or for example, a specificity of at least about 80% and sensitivity of at least about 95%).

Each biomarker listed in Tables 1, 2 and 3 is differentially present in biological samples derived from subjects having breast cancer as compared with normal subjects, and thus each is individually useful in facilitating the determination of breast cancer in a test subject. Such a method involves determining the level of the biomarker in a sample derived from the subject. Determining the level of the biomarker in a sample may include measuring, detecting, or assaying the level of the biomarker in the sample using any suitable method, for example, the methods set forth herein. Determining the level of the biomarker in a sample may also include examining the results of an assay that measured, detected, or assayed the level of the biomarker in the sample. The method may also involve comparing the level of the biomarker in a sample with a suitable control. A change in the level of the biomarker relative to that in a normal subject as assessed using a suitable control is indicative of the breast cancer status of the subject. A diagnostic amount of a biomarker that represents an amount of the biomarker above or below which a subject is classified as having a particular breast cancer status can be used. For example, if the biomarker is upregulated in samples derived from an individual having breast cancer as compared to a normal individual, a measured amount above the diagnostic cutoff provides a diagnosis of breast cancer. Generally, the individual small non-coding RNA biomarkers in Tables 1-3 are upregulated in breast cancer samples relative to samples obtained from normal individuals. As is well-understood in the art, adjusting the particular diagnostic cut-off used in an assay allows one to adjust the sensitivity and/or specificity of the diagnostic assay as desired. The particular diagnostic cut-off can be determined, for example, by measuring the amount of the biomarker in a statistically significant number of samples from subjects with different breast cancer statuses, and drawing the cut-off at the desired level of accuracy, sensitivity, and/or specificity. In certain embodiments, the diagnostic cut-off can be determined with the assistance of a classification algorithm, as described herein.

Accordingly, methods are provided for diagnosing breast cancer in a subject, by determining the level of at least one small non-coding RNA in a sample containing circulating small non-coding RNA from the subject, wherein a difference in the level of the at least one small non-coding RNA versus that in a normal subject (as determined relative to a suitable control) is indicative of breast cancer in the subject. In one embodiment, the at least one small non-coding RNA preferably includes one or more small non-coding RNAs from Table 1. In one embodiment, the at least one small non-coding RNA preferably includes one or more small non-coding RNAs from Table 2. In one embodiment, the at least one small non-coding RNA preferably includes one or more small non-coding RNAs from Table 3. For example, the present invention provides a method of determining the level of at least one small non-coding RNA in a sample containing circulating small non-coding RNA derived from the subject, wherein an increase in the level of the at least one small non-coding RNA relative to a control is indicative of breast cancer in the subject.

Optionally, the method may further comprise providing a diagnosis that the subject has or does not have breast cancer based on the level of at least one small non-coding RNA in the sample. In addition or alternatively, the method may further comprise correlating a difference in the level or levels of at least one small non-coding RNA relative to a suitable control with a diagnosis of breast cancer in the subject. In some embodiments, such a diagnosis may be provided directly to the subject, or it may be provided to another party involved in the subject's care.

While individual small non-coding RNA biomarkers are useful in diagnostic applications for breast cancer, as shown herein, a combination of small non-coding RNA biomarkers may provide greater predictive value of breast cancer status than the small non-coding RNA biomarkers when used alone. Specifically, the detection of a plurality of small non-coding RNA biomarkers can increase the accuracy, sensitivity, and/or specificity of a diagnostic test. Exemplary small non-coding RNA biomarkers and biomarker combinations are shown in Table 1. Exemplary small non-coding RNA biomarkers and biomarker combinations are shown in Table 2. Exemplary small non-coding RNA biomarkers and biomarker combinations are shown in Table 3. The invention includes the individual biomarkers and biomarker combinations as set forth in these tables, and their use in methods and kits described herein.

Accordingly, methods are provided for diagnosing breast cancer in a subject, by determining the level of two or more small non-coding RNAs in a sample containing circulating small non-coding RNA from the subject, wherein a difference in the level of the small non-coding RNAs versus that in a normal subject (as determined relative to a suitable control) is indicative of breast cancer in the subject. In one embodiment, the small non-coding RNAs preferably include one or more of a small non-coding RNA shown in Table 1. In one embodiment, the small non-coding RNAs preferably include one or more of a small non-coding RNA shown in Table 2. In one embodiment, the small non-coding RNAs preferably include one or more of a small non-coding RNA shown in Table 3.

Also provided is a method of diagnosing breast cancer in a subject by determining the levels of two or more small non-coding RNAs in a sample containing circulating small non-coding RNA from the subject, comparing the levels of the two or more small non-coding RNAs in the sample to a set of data representing levels of the same small non-coding RNAs present in normal subjects and subjects having breast cancer, and diagnosing the subject as having or not having breast cancer based on the comparison. In such a method, the set of data serves as a suitable control or reference standard for comparison with the sample from the subject.

Comparison of the sample from the subject with the set of data may be assisted by a classification algorithm, which computes whether or not a statistically significant difference exists between the collective levels of the two or more small non-coding RNAs in the sample, and the levels of the same small non-coding RNAs present in normal subjects or subjects having breast cancer.

### Generation of Classification Algorithms for Qualifying Cancer Status

In some embodiments, data that are generated using samples such as “known samples” can then be used to “train” a classification model. A “known sample” is a sample that has been pre-classified, e.g., classified as being derived from a normal subject, or from a subject having breast cancer. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

In some embodiments, data for the training data set that is used to form the classification model can be obtained directly from quantitative PCR (for example, Ct values obtained using the double delta Ct method), or from high-throughput expression profiling, such as microarray analysis (for example, total counts or normalized counts from a small non-coding RNA expression assay).

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm. Learning algorithms asserted for use in classifying biological information are described, for example, in PCT International Publication No. WO 01/31580 (Barnhill et al., “Methods and devices for identifying patterns in biological systems and methods of use thereof”), U.S. patent application No. 2002 0193950 A1 (Gavin et al, “Method or analyzing mass spectra”), U.S. patent application No. 2003 0004402 A1 (Hitt et al., “Process for discriminating between biological states based on hidden patterns from biological data”), and U.S. patent application No. 2003 0055615 A1 (Zhang and Zhang, “Systems and methods for processing biological expression data”). The contents of the foregoing patent applications are incorporated herein by reference in their entirety.

The classification models can be formed on and used on any suitable digital computer. Suitable digital computers include micro, mini, or large computers using any standard or specialized operating system, such as a Unix, WINDOWS or LINUX based operating system.

The training data set(s) and the classification models can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above can be used for developing classification algorithms for small non-coding RNA biomarkers for breast cancer. The classification algorithms can, in turn, be used in diagnostic tests by providing diagnostic values (e.g., cut-off points) for biomarkers used singly or in combination.

### Additional Diagnostic Tests

The level of small non-coding RNA biomarkers indicative of breast cancer, may be used as a stand-alone diagnostic indicator of breast cancer, in a subject. Optionally, the methods may include the performance of at least one additional test to facilitate the diagnosis of breast cancer. For example, other tests in addition to determining the level of one or more small non-coding RNA biomarkers in order to facilitate a diagnosis of breast cancer, may be performed. Any other test or combination of tests used in clinical practice to facilitate a diagnosis of breast cancer, may be used in conjunction with the small non-coding RNA biomarkers described herein.

### Methods of Treatment

In some embodiments, where a subject is diagnosed with breast cancer by the methods described herein, the present invention further provides methods of treating such subjects identified to have breast cancer. Accordingly, in one embodiment, the invention relates to a method of treating breast cancer in a subject, comprising determining the level of at least one small non-coding RNA biomarker in a sample derived from the subject, wherein a difference in the level of at least one small non-coding RNA biomarker versus that in a normal subject as determined relative to a suitable control is indicative of breast cancer in the subject, and administering a therapeutically effective amount of a breast cancer therapeutic to the subject. In another embodiment, the invention relates to a method of treating a subject having breast cancer, comprising identifying a subject having breast cancer in which the level of at least one small non-coding RNA biomarker in a sample derived from the subject is different (e.g., increased) versus that in a normal subject as determined relative to a suitable control, and administering a therapeutically effective amount of a breast cancer therapeutic to the subject.

The term “breast cancer therapeutic” includes, for example, substances approved by the U.S. Food and Drug Administration for the treatment of breast cancer. Drugs approved to treat breast cancer include, but are not limited to, Abemaciclib, Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Capecitabine, Clafen (Cyclophosphamide), Cyclophosphamide, Cytoxan (Cyclophosphamide), Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib, Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Neratinib Maleate, Nerlynx (Neratinib Maleate), Nolvadex (Tamoxifen Citrate), Paclitaxel, Paclitaxel Albumin-stabilized Nanoparticle Formulation, Palbociclib, Pamidronate Disodium, Perjeta (Pertuzumab), Pertuzumab, Ribociclib, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Verzenio (Abemaciclib), Vinblastine Sulfate, Xeloda (Capecitabine), Zoladex (Goserelin Acetate).

The breast cancer therapeutics may be administered to a subject using a pharmaceutical composition. Suitable pharmaceutical compositions comprise a pharmaceutically effective amount of a breast cancer therapeutic (or a pharmaceutically acceptable salt or ester thereof), and optionally comprise a pharmaceutically acceptable carrier). In certain embodiments, these compositions optionally further comprise one or more additional therapeutic agents.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66:1-19 (1977), incorporated herein by reference. The salts can be prepared in situ during the final isolation and purification of the compounds of the invention, or separately by reacting a free base or free acid function with a suitable reagent. For example, a free base function can be reacted with a suitable acid. Furthermore, where the compounds carry an acidic moiety, suitable pharmaceutically acceptable salts thereof may, include metal salts such as alkali metal salts, e.g. sodium or potassium salts; and alkaline earth metal salts, e.g. calcium or magnesium salts.

The term “pharmaceutically acceptable ester”, as used herein, refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms.

As described above, the pharmaceutical compositions may additionally comprise a pharmaceutically acceptable carrier. The term carrier includes any and all solvents, diluents, or other liquid vehicle, dispersion or suspension aids, surface active agents, isotonic agents, thickening or emulsifying agents, preservatives, solid binders, lubricants and the like, suitable for preparing the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Compositions for use in the present invention may be formulated to have any concentration of the breast cancer therapeutic desired. In preferred embodiments, the composition is formulated such that it comprises a therapeutically effective amount of the breast cancer therapeutic.

The disclosure generally relates to a method of diagnosing a subject with a benign, pre-malignant, or malignant hyperproliferative cell comprising: detecting the presence, absence, and/or quantity of at least one non-coding RNA or functional fragment thereof in a sample. In some embodiments, the step of detecting comprise exposing a sample from a subject (e.g. a human subject), to one or a plurality of probes, each probe capable of binding one or a plurality of non-coding RNA molecules in the sample. In some embodiments, the probe is a nucleic acid molecule (DNA, RNA or hybrid thereof) that comprises at least 70%, 80%, 85%, 90%, 95%, 96%, 97%, 98%, 99% sequence homology or sequence identity to any nucleic acid sequences of Tables 1, 2, and/or 3. In some embodiments, the probe is a nucleic acid molecule (DNA, RNA or hybrid thereof) that comprises at least 70%, 80%, 85%, 90%, 95%, 96%, 97%, 98%, 99% sequence homology or sequence identity to SEQ ID NO:3, SEQ ID NO: 19, SEQ ID NO:32, SEQ ID NO:40, SEQ ID NO:41, SEQ ID NO: 79, SEQ ID NO:82, SEQ ID NO:83, SEQ ID NO: 126, SEQ ID NO:148, or SEQ ID NO:191. In some embodiments, the probe is a nucleic acid molecule (DNA, RNA or hybrid thereof) that is an RNA sequence comprising at least 70%, 80%, 85%, 90%, 95%, 96%, 97%, 98%, 99% sequence homology or sequence identity to SEQ ID NO: 3, SEQ ID NO: 19, SEQ ID NO:32, SEQ ID NO:40, SEQ ID NO:41, SEQ ID NO: 79, SEQ ID NO:82, SEQ ID NO:83, SEQ ID NO: 126, SEQ ID NO: 148, or SEQ ID NO: 191, where each thymine is replaced with a uracil. In some embodiments, the plurality of probes are one or a combination of nucleic acid sequences that are an RNA complementary to the a nucleic acid sequence comprising at least 70%, 80%, 85%, 90%, 95%, 96%, 97%, 98%, 99% sequence homology or sequence identity to SEQ ID NO:3, SEQ ID NO: 19, SEQ ID NO:32, SEQ ID NO: 40, SEQ ID NO:41, SEQ ID NO: 79, SEQ ID NO:82, SEQ ID NO:83, SEQ ID NO: 126, SEQ ID NO: 148, or SEQ ID NO: 191. In some embodiments, the plurality of probes are one or a combination of nucleic acid sequences chosen from: SEQ ID NO:3, SEQ ID NO: 19, SEQ ID NO: 32, SEQ ID NO:40, SEQ ID NO:41, SEQ ID NO: 79, SEQ ID NO: 82, SEQ ID NO: 83, SEQ ID NO: 126, SEQ ID NO: 148, or SEQ ID NO: 191. In some embodiments, the plurality of probes are one or a combination of nucleic acid sequences complementary to the nucleic acid sequences chosen from: SEQ ID NO:3, SEQ ID NO: 19, SEQ ID NO:32, SEQ ID NO:40, SEQ ID NO: 41, SEQ ID NO:79, SEQ ID NO:82, SEQ ID NO:83, SEQ ID NO: 126, SEQ ID NO: 148, or SEQ ID NO: 191.

In any of the disclosed method embodiments, the subject may be a human diagnosed with or suspected as having a breast cancer. In any of the disclosed method embodiments, wherein the step of detecting is preceded by a step of acquiring a sample from the subject.

In some embodiments, the probe or plurality of probes are one or a plurality of antibodies or antibody fragments comprising a CDR that binds to a nucleic acid molecule (DNA, RNA or hybrid thereof) that comprises at least 70%, 80%, 85%, 90%, 95%, 96%, 97%, 98%, 99% or 100% sequence homology or sequence identity to SEQ ID NO:3, SEQ ID NO: 19, SEQ ID NO: 32, SEQ ID NO:40, SEQ ID NO:41, SEQ ID NO: 79, SEQ ID NO:82, SEQ ID NO: 83, SEQ ID NO: 126, SEQ ID NO:148, or SEQ ID NO:191. In some embodiments, the probe or plurality of probes are one or a plurality of antibodies or antibody fragments comprising a CDR that binds to a nucleic acid molecule (DNA, RNA or hybrid thereof) that comprises at least 70%, 80%, 85%, 90%, 95%, 96%, 97%, 98%, 99% or 100% sequence homology or sequence identity to SEQ ID NO:3, SEQ ID NO: 19, SEQ ID NO:32, SEQ ID NO:40, SEQ ID NO:41, SEQ ID NO: 79, SEQ ID NO:82, SEQ ID NO:83, SEQ ID NO: 126, SEQ ID NO: 148, or SEQ ID NO: 191, wherein each of sequences are modified such that the thymines in each of SEQ ID NO: 3, SEQ ID NO: 19, SEQ ID NO:32, SEQ ID NO:40, SEQ ID NO:41, SEQ ID NO: 79, SEQ ID NO: 82, SEQ ID NO:83, SEQ ID NO: 126, SEQ ID NO: 148, or SEQ ID NO: 191 are replaced with a uracil. In some of the embodiments, the methods further comprise isolating RNA from the sample before exposing the sample to one or a plurality of probes. In some embodiments, the method comprises detecting or quantifying an amount of non-coding RNAs, such as small RNAs (smRNAs), in a sample by performing semiquantitative or quantitative PCR or sequencing analysis of the non-coding RNAs in a sample. Probes may be immobilized to a solid support such as an ELISA plate, plastic, slide, microarray, silica chip or other surface such that the single-strand nucleotide sequences are exposed to a sample comprising non-coding RNAs from a subject. The probes may comprise, in some embodiments, from about 5 to bout 100 nucleotides in length and comprise any of the sequences in Tables 1, 2, and/or 3 or any complementary sequence in RNA or DNA of the sequences set forth in Tables 1, 2, and/or 3. In any of the disclosed method embodiments, the step of detecting the presence, absence, and/or quantity of at least one non-coding RNA or homologous sequence thereof at least 70% homolgous to one of the noncoding RNAs in a sample comprises using a chemiluminescent probe, fluorescent probe, and/or fluorescence microscopy, calculating the presence or quantity by correlating the signal of the dectable probe to the presence of the non-coding RNA.

The disclosure generally relates detecting the presence of T3p in a sample and correlating the presence of the T3p in the sample with the presence of breast cancer. The disclosure also relates to detecting the presence of a nucleic acid molecule (DNA, RNA or hybrid thereof) that comprises, consists of or consists essentially of at least 70%, 80%, 85%, 90%, 95%, 96%, 97%, 98%, 99% or 100% sequence homology or sequence identity to SEQ ID NO:3, SEQ ID NO: 19, SEQ ID NO:32, SEQ ID NO:40, SEQ ID NO:41, SEQ ID NO: 79, SEQ ID NO:82, SEQ ID NO:83, SEQ ID NO: 126, SEQ ID NO: 148, or SEQ ID NO: 191 or an RNA molecule thereof wherein one or more of the thymines in any of the sequence identifiers are replaced by uracil. In some embodiments, the probe or plurality of probes on a solid support comprise a sequence complementary to SEQ ID NO:3, SEQ ID NO: 19, SEQ ID NO:32, SEQ ID NO:40, SEQ ID NO:41, SEQ ID NO: 79, SEQ ID NO:82, SEQ ID NO:83, SEQ ID NO: 126, SEQ ID NO: 148, or SEQ ID NO: 191 that is from about 5 to about 1000 nucleotides in length. In some embodiments, the probe or plurality of probes on a solid support comprise a sequence complementary to SEQ ID NO:3, SEQ ID NO: 19, SEQ ID NO:32, SEQ ID NO:40, SEQ ID NO: 41, SEQ ID NO: 79, SEQ ID NO: 82, SEQ ID NO: 83, SEQ ID NO: 126, SEQ ID NO: 148, or SEQ ID NO: 191 that is from about 5 to about 500 nucleotides in length. In some embodiments, the probe or plurality of probes on a solid support comprise a sequence complementary to SEQ ID NO: 3, SEQ ID NO:19, SEQ ID NO:32, SEQ ID NO:40, SEQ ID NO:41, SEQ ID NO: 79, SEQ ID NO:82, SEQ ID NO:83, SEQ ID NO: 126, SEQ ID NO: 148, or SEQ ID NO: 191 that is from about 5 to about 100 nucleotides in length. In some embodiments, the probe or plurality of probes on a solid support comprise a sequence complementary to SEQ ID NO:3, SEQ ID NO:19, SEQ ID NO:32, SEQ ID NO:40, SEQ ID NO:41, SEQ ID NO: 79, SEQ ID NO:82, SEQ ID NO: 83, SEQ ID NO:126, SEQ ID NO: 148, or SEQ ID NO: 191 that is from about 5 to about 50 nucleotides in length.

In some embodiments, any of the methods disclosed herein further comprise a step of correlating the presence or quantity of non-coding RNAs such as those disclosed in Tables 1, 2, and/or 3 or any combination thereof to the likelihood that the subject has cancer, such as breast cancer.

### Kits for Detection Small RNA Biomarkers

In another aspect, the present invention provides kits for diagnosing breast cancer status in a subject, which kits are useful for determining the level of one or more of a small non-coding RNA biomarkers from Table 1, Table 2 or Table 3 (wherein the sequences optionally comprise uracils in place of one, more than one or all of the disclosed thymines), and combinations thereof. In one embodiment, the one or more small non-coding RNAs are selected from the biomarkers listed in Table 1. In one embodiment, the one or more small non-coding RNAs are selected from the biomarkers listed in Table 2. In one embodiment, the one or more small non-coding RNAs are selected from the biomarkers listed in Table 3. Kits may include materials and reagents adapted to selectively detect the presence of a small non-coding RNA or group of small non-coding RNAs diagnostic for breast cancer in a sample derived from a subject. For example, in one embodiment, the kit may include a reagent that specifically hybridizes to a small non-coding RNA. Such a reagent may be a nucleic acid molecule in a form suitable for detecting the small non-coding RNA, for example, a probe or a primer. The kit may include reagents useful for performing an assay to detect one or more small non-coding RNAs, for example, reagents which may be used to detect one or more small non-coding RNAs in a qPCR reaction. The kit may likewise include a microarray useful for detecting one or more small non-coding RNAs.

In a further embodiment, the kit may contain instructions for suitable operational parameters in the form of a label or product insert. For example, the instructions may include information or directions regarding how to collect a sample, how to determine the level of one or more small non-coding RNA biomarkers in a sample, or how to correlate the level of one or more small non-coding RNA biomarkers in a sample with the breast cancer status of a subject.

In another embodiment, the kit can contain one or more containers with small non-coding RNA biomarker samples, to be used as reference standards, suitable controls, or for calibration of an assay to detect the biomarkers in a test sample.

Other embodiments are described in the following non-limiting Examples. Various publications, including patents, published applications, technical articles and scholarly articles are cited throughout the specification. Each of these cited publications is incorporated by reference herein in its entirety.

### EXAMPLES

### Example 1. Methods

Examples 2-6 were carried out with methods including, but not limited to, the following:

### Tissue Culture

MDA-MB-231 and MDA-LM2 cells were cultured in Dulbecoco's medium supplemented with 10% fetal bovine serum, L-glutamine, sodium pyruvate, penicillin-streptomycin, and amphotericin. Cell lines were obtained from American Type and Culture Collection (ATCC) and were grown according to protocol.

All cells were cultured in a 37° C., 5% CO₂ humidified incubator. Cell lines MDA-MB-231, MDA-LM2, CN34-par, CN34-Lm1a, MCF7 and MDA-MB-453 were propagated in DMEM base media supplemented with 4.5 g/L glucose, 10% FBS, 4 mM L-glutamine, 1 mM sodium pyruvate, penicillin (100 units/mL), streptomycin (100 μg/mL) and amphotericin (1 μg/mL). Cell lines HCC1395, ZR-75-1 and HCC38 were propagated in RPMI 1640 base media supplemented with 10% FBS, 2 mM L-glutamine, penicillin (100 units/mL), streptomycin (100 μg/mL) and amphotericin (1 μg/mL). SK-BR-3 cell line was propagated in McCoy's 5a modified media supplemented with 10% FBS, penicillin (100 units/mL), streptomycin (100 μg/mL) and amphotericin (1 μg/mL). HMECs were obtained from Thermo Fisher Scientific and propagated in HuMEC ready media (Thermo Fisher Scientific).

Small and Exosomal RNA Extraction and Sequencing: Preparation of conditioned media for isolation of RNA from extracellular vesicle and total conditioned media was carried out by seeding cells at 7×10⁵. 24 hours later, cells were washed twice with PBS and 10 mL exosome-depleted media was added. 48 hours later, media was harvested by spinning at 200×g for 15 minutes and taking the supernatant. Exosome-depleted media was prepared by substituting exosome-depleted FBS (Thermo Fisher Scientific) for FBS. Exosome-depleted HMEC media was prepared by centrifuging the bovine pituitary extract media component at 100,000×g at 4° C. for 16 hours.

Extracellular vesicle RNA was isolated from 5 mL conditioned media, prepared as outlined above, using the Cell Culture Media Exosome Purification and RNA Isolation kit (Norgen Biotek). RNA from conditioned media was isolated from 400 ul total conditioned media using the miRNeasy serum/plasma kit (Qiagen). Total cellular small RNA samples were extracted using Norgen Biotek small RNA purification kit according to the manufacturer's protocol. RNA samples were subsequently prepared for high-throughput sequencing with the NEXTflex Small RNA Sequencing Kit v3 using the manufacturer's protocol (Bioo Scientific). The resulting libraries were then sequenced and processed as recommended by the manufacturer. Briefly, cutadapt (v1.4) was used to remove the adapter sequences and trim the degenerate sequences at the beginning and end of each read. We then used bowtie2 (v2.3.3) to align the resulting sequences to the human genome (build hg38). The resulting BAM files were then sorted and converted to BED for further analysis. Extracellular vesicle RNA was isolated from serum samples using the Plasma/serum Exosome Purification and RNA isolation kit (Norgen Biotek) according to the manufacturer's instructions.

TCGA-BRCA small RNA sequencing data and identification of oneRNAs Reads from the TCGA-BRCA project were downloaded from the Genomic Data Commons (GDC) in BAM format (hg38) and the samples were annotated using the GDC API. Upon conversion to the BED format, the Piranha package⁴⁰ was used to identify the expressed small RNA loci. The resulting loci were merged across all samples using mergeBed to create a comprehensive list of small RNA loci expressed in breast tissue and breast cancer.

By enumerating the small RNA sequences obtained from breast cancer cell lines and HMECs, we generated a count table for each small RNA locus. We then normalized the resulting table by library size and retained only those loci with no observed reads across the three HMEC replicates. We used two independent statistical tests to compare either all cancer cell lines or each subtype individually (TNBC, HER2+, and Luminal): (i) we used the DESeq package in R to calculate an adjusted p-value, and (ii) we used Fisher's exact test to compare presence and absence of each small RNA. We selected those loci with either an adjusted P<0.05 in the former or P<0.1 in the latter test across all comparisons.

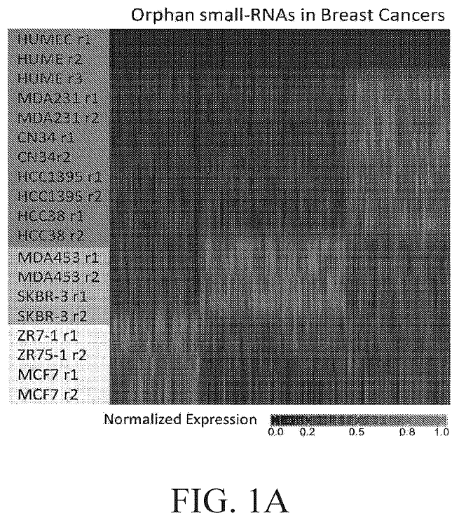

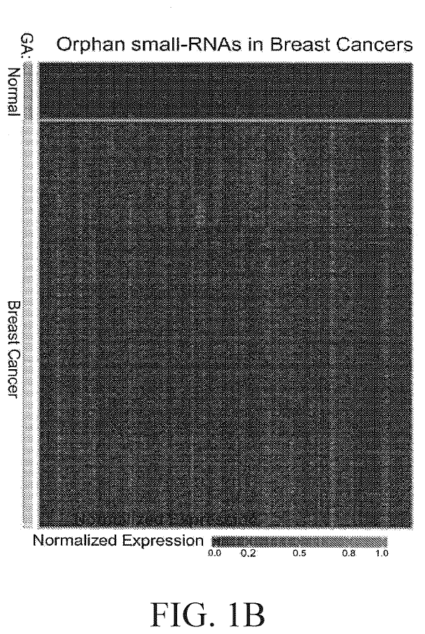

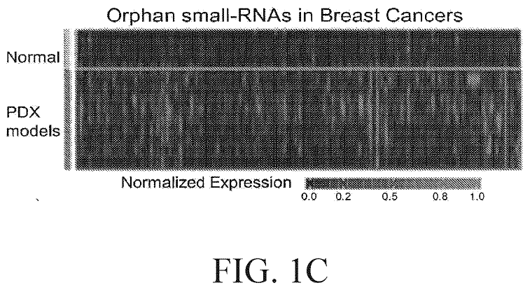

Four thirty-seven loci, listed in FIG. 1A, satisfied these criteria. For visualization, we max-normalized each row and performed a k-means clustering (k=3). For the TCGA-BRCA database, we generated a similar count table across all subtype annotated samples (based on PAM50 classification) and all small RNA loci and normalized the resulting table to generate a count-per-million reads (cpm) table. In order to identify ‘orphan’ small RNAs, i.e. small RNAs that are largely absent in normal cells, we first retained only the loci with their 90^{th} percentile expression in normal samples below 0.5 cpm. Of the 437 loci above, 268 passed this step. We then performed Fisher's exact tests to compare the presence of all small RNAs across the tumor samples and normal biopsies. We performed similar comparisons between normal samples and each of the breast cancer subtypes. We then retained those loci that were significant in at least one of these tests with an adjusted p-value of <0.05. 201 of small RNAs satisfied this final step and were thus classified as orphan non-coding RNAs. We confirmed that none of these small RNAs were previously annotated as miRNAs, snoRNA, or tRNAs.

### Small RNA Sequencing of PDX Models and Normal Epithelial Samples:

All human samples used to generate PDX tumors, as well as the human non-tumor samples, were previously described⁴¹. Small RNA profiling and data pre-processing was carried out by Q²Solutions. The abundance of oncRNAs in these samples was determined as described above. Comparing oncRNA expression between poorly and highly metastatic cells We used the R package DESeq2 to compare expression of oncRNAs between parental cell lines in FIG. 1 (MDA231 and CN34) and their highly metastatic in vivo selected derivatives (MDA-MB-231 background) to identify those oncRNAs that were significantly upregulated in highly metastatic cells. We identified T3p in this analysis, which we also confirmed in a small RNA dataset we had previously generated for these lines⁷. In addition, we also performed quantitative RT-PCR assays. For this, we extracted small RNAs from MDA-231 parental cells and their highly metastatic MDA-LM2 (microRNA Purification Kit; Norgen) and performed stem-loop qPCR, using the following primers: R: 5′-CCAGTGCAGGGTCCGAGGTA and F: 5′-CCCAGGACTCGGCTCACAC.

### T3p Expression and Clinical Association in the TOGA-BRCA Dataset

We used the metadata accompanying the TCGA-BRCA dataset to perform survival analysis based on T3p expression in tumor samples. We stratified the patients based on T3p levels and generated Kaplan-Meier curves using all tertiles and performed log-rank (Mantel-Cox) test to calculate the associated p-value. We similarly used the clinical data to compare T3p expression across early and late stage tumors (one-tailed Mann-Whitney U-test).

T3p modulation and gene expression profiling: We used miRCURY LNA inhibitors (Exiqon) against the following sequences: T3p: CAGGACTCGGCTCACACATGC; TERC: TTGTCTAACCCTAACTGAGAAGG; Scrambled: AGACGACAGCTGGATCACACG. Similarly, we used T3p mimetics (IDT): rC*rArGrGrArCrUrCrG rGrCrUrCrArCrArCrArUrG*rC (T3p mimetic) and rA*rGrA rCrGrA rCrArG rCrUrG rGrArU rCrArC rArC*rG (control). We then transfected the LNAs in the highly metastatic MDA-LM2 cells and the mimetics in the parental MDA-MB-231 cells and performed gene expression profiling as previously described⁷. Differential gene expression analysis was also performed as previously described⁷.

### Tough Decoys and In Vivo Lung Colonization and Tumor Growth Assays

MDA-LM2 cells were transfected with anti-T3p or scrambled LNA (same as above) and after 48 hours, cells were injected via tail-vein into the vasculature of immunocompromised NOD SCID gamma (NSG) mice (2.5×10⁴ per mouse; n=5 per cohort). In vivo imaging and comparison of curves was performed as previously described¹⁹. Lungs from at least three mice per cohort (middle signal) were then extracted, fixed, sectioned, stained (H&E) and quantified as previously described¹⁹. To generate stable inhibition of T3p, we designed TuDs against this small RNA in a lentiviral backbone under a RNA PolIII promoter (pLKO.1). We then stably transduced MDA-LM2 cells and performed lung colonization assays (same as above; 5×10⁴ cells per mouse). HCC1395 cells were similarly transduced and injected at 2×10⁵ cells per mouse. Orthotopic tumor growth assays were performed by injecting 2.5×10⁵ cells resuspended in 50 μl PBS mixed with 50 μl matrigel into the mammary glands of age-matched 6-8 week old female NOD/SCID gamma mice using a 28 gauge needle. Tumor volume was determined by using calipers to measure the tumor length (L) and width (W) every two days and calculated using the formula πLW2/6. The experiment endpoint was reached once tumors reached a volume of 800 mm³. Cell proliferation In vitro: cancer cell proliferation assays are performed by seeding 5×10⁴ cells at day 0 and then counting them in triplicate on day 3 and day 5. The slope of the best fitted line, estimated using linear models, between log of cell counts and days is the reported proliferation rate (days⁻¹). For cell-cycle analysis, cells were grown to 80% confluency in 6 cm plates, harvested and fixed in 70% ethanol. Cells were then pelleted and resuspended in 50 μg/mL propidium iodide (Thermo Fisher Scientific) and 1 mg/mL RNase A (Thermo Fisher Scientific) and allowed to incubate 1 hour at 37° C. BD Aria2 flow cytometer was then used for FACS analysis; post-FACS analysis and cell cycle quantification was performed using the python package ‘fcsparser’. FCSparser: https://github.com/eyurtsev/fcsparser/tree/master/fcsparser

Co-expression analysis for finding T3p biogenesis factors: In order to identify the regulators of T3p biogenesis, we listed the genes with known nuclease activity (GO:0004540 and GO:0004525) and further added RNA-binding proteins that are known to interact with these nucleases⁴². We then performed co-expression analysis between T3p levels and those of the genes on this list across the TCGA-BRCA dataset. We overlapped the genes with strong associations with those that are upregulated in highly metastatic MDA-LM2 cells and are similarly higher in breast cancer samples relative to normal biopsies in the TCGA-BRCA dataset. Based on these criteria, we identified seven candidates, two of which had known double-stranded binding activity. Since the CR7 domain of TERC is structured (i.e. form double-stranded regions), we reasoned that these proteins, namely DROSHA and TARBP2, were the best candidates for follow-up. We used siRNAs to knockdown DROSHA and TARBP2, and also DGCR8 and DICER1 that are known to interact with these proteins respectively (IDT). We used the following target sequences: TARBP2: 5′-ACCTGGGATTCTCTACGAAATTCAGT, DROSHA: 5′-CCTTGATTGAGGTATAGTTCTTGTCT, DICER1: 5′-TGGTGCTTAGTAAACTCTTGGTTCCA, and DGCR8: 5′-CTGCAGGAGTAAGGACAGGAAGGTGC. Following siRNA transfection and knockdown verification, we performed small RNA sequencing as described above.

Training and testing of oncRNA-based classifiers: Of the 201 oncRNAs, 100 were detected in at least one serum sample. We used these 100 oncRNAs to train a GBC on subtype-annotated TCGA-BRCA samples (sklearn module). We then bootstrapped our compendium of serum samples from 35 healthy and 40 cancer patients 100 times to calculate the performance parameters of the classifier, namely average AUROC, precision, and accuracy scores. We also performed an independent assessment of oncRNAs by performing training and testing on the serum data as opposed to TCGA-BRCA (5-fold cross-validation). We used the following miRNAs to perform similar analyses as above: miR-10b-5p, miR-10b-3p, miR-148b-3p, miR-148b-5p, miR-155-3p, miR-155-5p, miR-34a-3p, miR-376a-3p, miR-652-3p, miR-133a-3p, miR-139-3p, miR-143-3p, miR-145-3p, miR-15a-3p, miR-18a-3p, miR-425-3p, miR-34a-5p, miR-376a-5p, miR-652-5p, miR-133a-5p, miR-139-5p, miR-143-5p, miR-145-5p, miR-15a-5p, miR-18a-5p, miR-425-5p, miR-127-3p, miR-194-5p, miR-205-5p, miR-21-5p, miR-375, miR-376c-3p, miR-382-5p, miR-409-3p, and miR-411-5p. Animal Studies: All animal studies were completed according to University of California San Francisco IACUC guidelines. Statistical Methods: Statistical tests used to assess data significance are described in the legends. Briefly, unless otherwise stated, we have used non-parametric tests to perform pair-wise comparisons. For mouse experiments, we have used two-way ANOVA with time as a co-variate. For proliferation rates, we have used linear models. The analyses were performed in python, R, and prism environments.

### Example 2. A Systematic Search for Orphan Small Non-Coding RNAs in Breast Cancer

To search for a new class of cancer-specific small-RNAs that are expressed in breast cancer cells, yet are undetectable in normal breast tissue, an unbiased approach was used based on small RNA sequencing of multiple breast cancer subtypes as well as human mammary epithelial cells. Roughly 200 previously unknown small RNAs were discovered and annotated that are specifically expressed in breast cancer cells. Borrowing terminology from bacterial genetics, these RNAs have been named ‘orphan’ non-coding RNAs (oncRNAs) to highlight their cancer-specific biogenesis.

It was first determined if a set of small RNAs exists that are only present in cancer cells and can provide an accessible pool of potential regulators in these cells. It was reasoned that such oncRNAs would only be detectable in cancer cell lines and not in normal cells. To test this hypothesis, small RNA sequencing was performed on nine breast cancer cell lines (representing all major breast cancer subtypes) as well as human mammary epithelial cells (HMEC) as a reference. 437 unannotated small RNAs were identified that were significantly detected across all of the breast cancer lines while remaining undetected in HMEC samples (FIG. 1A).

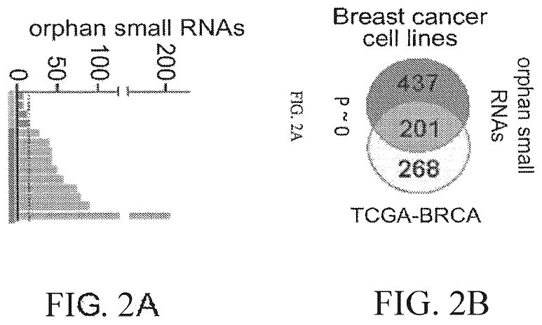

To further narrow the search and strengthen these findings, a similar analysis was performed on the small RNA sequencing data obtained from The Cancer Genome Atlas (TCGA), which provided small RNA expression profiles across roughly 200 normal tissue samples and 1200 breast cancer biopsies. In this analysis, 268 cancer-specific small RNAs were identified, 201 of which were also present in the analysis of breast cancer lines. The highly significant overlap between these two independent analyses revealed a high-confidence set of 201 oncRNAs (FIG. 1B and FIG. 2A), shown in Table 1, below.

| Chromo- | Start on | End on | | Chromo- | SEQ | |
|---|---|---|---|---|---|---|
| some | Chromo- | Chromo- | OncRNA | some | ID | |
| Location | some | some | ID | strand | NO | Nucleotide Sequence |
| chr1 | 6554450 | 6554500 | tag_281 | − | 1 | GGCGGACTCGGGACTGCTGCTAAAGCGGG |
| | | | | | | GTTCCTGCCGTTCCACTTGGC |
| chr1 | 9690200 | 9690250 | tag_390 | + | 2 | GGGTGGCAGACCCAGATCCTGAGGTCCTG |
| | | | | | | CGGCTTCCTCCCCGGGGGAGC |
| chr1 | 20787250 | 20787300 | tag_860 | − | 3 | GGCGGCTGGGACTGGAGGACAGCGGTGGC |
| | | | | | | GGAGGCGACTAGCGGCGGCGG |
| chr1 | 28914600 | 28914650 | tag_1217 | + | 4 | GCTCGCTCGCTCCCTCCCTCCGCTGGTGC |
| | | | | | | GTTTAGTCAGTCAGCCAGCAG |
| chr1 | 55126400 | 55126450 | tag_2286 | + | 5 | GCTTCCTTCTTTATTTTTCTCTACAGCTC |
| | | | | | | TCCTCACCATCTGACACATAC |
| chr1 | 103470900 | 103470950 | tag_3856 | + | 6 | TTTTACAAGCCATTTCTGAGATGGGAAAC |
| | | | | | | TATTAGGTCAGTGCAAAAGTG |
| chr1 | 110019500 | 110019550 | tag_4074 | + | 7 | GTCTAAGAAATATTTTTGTGCTTTCTGGC |
| | | | | | | CTTCTTTTCCCACCTTAGGCT |
| chr1 | 120723950 | 120724000 | tag_4510 | + | 8 | ACTTCCGGCGGCGGCTGAGGCGGCGGCCG |
| | | | | | | AGGAGCGGCGGACTCGGGGCG |
| chr1 | 149888200 | 149888250 | tag_4908 | + | 9 | TCAAACAAAATACTGGTTTTGTTTTACTG |
| | | | | | | AGAGGCACTGTGGGTTTTTGT |
| chr1 | 151372650 | 151372700 | tag_5001 | − | 10 | AGCGGCCTCTGACACCAGCACAGCAAACC |
| | | | | | | CGCCGGGATCAAAGTGTACCA |
| chr1 | 153968100 | 153968150 | tag_5136 | − | 11 | CGGTAGGGCCGCTGTATCTGGGAGTAGGG |
| | | | | | | GACTAAGAGTCTGAGGGTCCA |
| chr1 | 154975050 | 154975100 | tag_5206 | + | 12 | TACTGACCACCCTCACCCATGAGGCTTTC |
| | | | | | | TGGATCATTCTCTGAACTTTA |
| chr1 | 155063750 | 155063800 | tag_5214 | + | 13 | TTGTACCTTTCTCTCCTCGACTGTGAAGC |
| | | | | | | GGGCCGGGACCTGCCAGGCCA |
| chr1 | 162157100 | 162157150 | tag_5638 | − | 14 | ATGGGGCTGGCTGGTTGTGGGATCTGGAG |
| | | | | | | GCATCTGGGGTTGGAATGTGA |
| chr1 | 162497850 | 162497900 | tag_5657 | + | 15 | GGCTGCTTGAAGTCCCGGGAGTCGGTGAG |
| | | | | | | GCGGCTGCAGGTCCCTCCCTG |
| chr1 | 164559150 | 164559200 | tag_5733 | + | 16 | CTGTATATGTTTCTGGAGTCCTGAGCCTG |
| | | | | | | AGCTAAACAAAAGCAGGAGGC |
| chr1 | 164589500 | 164589550 | tag_5737 | − | 17 | TCTGGAAGAAGTAATTTGCTCTATGGTTC |
| | | | | | | TCTGGTGTCCTGAAAAGTGTA |
| chr1 | 167935950 | 167936000 | tag_5869 | − | 18 | GGTTCACAGCGGGCAGGGAAAGCCGCGGG |
| | | | | | | AAGGGTACTCCAGGCGAGAGG |
| chr1 | 174159550 | 174159600 | tag_6119 | + | 19 | TGGCGGAGCGAACGGGACCGGCCCGGCTT |
| | | | | | | CAGAGCGCGAGGTGGAGGGTG |
| chr1 | 182589200 | 182589250 | tag_6440 | − | 20 | GCAGGAGGAAACTGCTCGGGCTGCAAGCA |
| | | | | | | GTCTTCCAGGCTTTGCGGCTG |
| chr1 | 211260850 | 211260900 | tag_7552 | − | 21 | CGCAAGCTGGGTTGGCAGAGGACGCTGGA |
| | | | | | | ACCTGGCTGGTCGGGGAGAAA |
| chr1 | 222618100 | 222618150 | tag_7990 | + | 22 | TGACCACAACATGGCTGCGGCGCCTGGGC |
| | | | | | | TGCTCGTCTGGCTGCTCGTGC |
| chr1 | 224378600 | 224378750 | tag_8061 | + | 23 | GGAACATCTGAATGCTGAGGCCTAGAGAT |
| | | | | | | CGTTCAGGGTGTTGTAACTGGGAACATCT |
| | | | | | | GAATGCTGAGGCCTAGAGATCGTTCAGGG |
| | | | | | | TGTTGTAACTGGGAACATCTGAATGCTGA |
| | | | | | | GGCCTAGAGATCGTTCAGGGTGTTGTAAC |
| | | | | | | TGGGA |
| chr1 | 226940300 | 226940350 | tag_8179 | + | 24 | TCCGGAGCGCGTGGCGGGCGTCAGCGCGG |
| | | | | | | TGGCCAGCGCGCAGAGGCGGG |
| chr1 | 228458100 | 228458150 | tag_8247 | + | 25 | GCGTTTCTGTTTGGAGAGACTCAGCCATC |
| | | | | | | ATGCCAGACCCGTCCAAATCG |
| chr1 | 235450000 | 235450050 | tag_8590 | − | 26 | TGTTTGCACAAATTTCCTTAAAAATCAAC |
| | | | | | | TTGTACTGTAGCATAAGAAAA |
| chr1 | 239459700 | 239459750 | tag_8751 | − | 27 | CTTTATGGCACTGGTAGACAAAACTATCA |
| | | | | | | ACTGTGTTAAAATAATTCTAG |
| chr1 | 245513450 | 245513500 | tag_8967 | − | 28 | CTGCCCTTATCAGTTTTGACCTGCTAGGT |
| | | | | | | GCTTCACAGAACTTTGCTTGA |
| chr10 | 8074750 | 8074900 | tag_9414 | + | 29 | TCATCATATTATACAGACCGAACTGTTGT |
| | | | | | | ATAAATTTATTTACTGCTAGTCTTAAGAA |
| | | | | | | CTGCTTTCTTTCGTTTGTTTGTTTCAATA |
| | | | | | | TTTTCCTTCTCTCTCAATTTTTGGTTGAA |
| | | | | | | TAAACTAGATTACATTCAGTTGGCCTAAG |
| | | | | | | GTGGT |
| chr10 | 42843200 | 42843250 | tag_10565 | + | 30 | CCAGCCTGAGCAACATAGCGAGACCCCGT |
| | | | | | | GTCTCTTTTTTTTTTTTTTTT |
| chr10 | 47570550 | 47570600 | tag_10732 | + | 31 | GTGGGAGGATCACTTGAGCCCGGAAGTTC |
| | | | | | | AAGACCACCCTAGGCAATATA |
| chr10 | 89306850 | 89306900 | tag_12154 | + | 32 | ATGAATCTAAGAGAGAATGGAATGTATGG |
| | | | | | | GAAAACAAAGTTACTGCAACT |
| chr10 | 98879600 | 98879650 | tag_12513 | + | 33 | AATAAATATGCATTCTCTTATTGGAGCAG |
| | | | | | | GCAGCCAGGAATGTAGAGCTC |
| chr11 | 1256600 | 1256650 | tag_13943 | + | 34 | GGACTCACGTGGATGACAG7GGAGGCCTC |
| | | | | | | CTGGATCTCTAGGTCTCAGGG |
| chr11 | 67464850 | 67464900 | tag_16543 | + | 35 | AGACACCTGCGGGAGGGACCAGAGCCCGG |
| | | | | | | TCAGGGCGAGGGGGCGGAGGC |
| chr11 | 70203300 | 70203350 | tag_16694 | + | 36 | TGTCGATTTCCTGTAGTGAATCAGGCACC |
| | | | | | | GGAGTGCAGGTTCGGGGGTGG |
| chr11 | 112677900 | 112677950 | tag_18237 | | 37 | CCCCCCGCCACTGCTGAAT7TGACTGGCT |
| | | | | | | ATAAAAATAAAATAAAATCCA |
| chr11 | 134609150 | 134609200 | tag_19163 | − | 38 | TCCTTGATGTATAAAAATTAACAAAAATA |
| | | | | | | ATTTTACTTGTGGCAATGTTC |
| chr12 | 6537600 | 6537950 | tag_19454 | + | 39 | CACTGCCACCCAGAAGACTGTGGATGGCC |
| | | | | | | CCTCCGGGAAACTGTGGCGTGATGGCCGC |
| | | | | | | GGGGCTCTCCAGAACATCATCCCTGCCTC |
| | | | | | | TACTGGCGCTGCCAAGGCTGTGGGCAAGG |
| | | | | | | TCATCCCTGAGCTGAACGGGAAGCTCACT |
| | | | | | | GGCATGGCCTTCCGTGTCCCCACTGCCAA |
| | | | | | | CGTGTCAGTGGTGGACCTGACCTGCCGTC |
| | | | | | | TAGAAAAACCTGCCAAATATGATGACATC |
| | | | | | | AAGAAGGTGGTGAAGCAGGCGTCGGAGGG |
| | | | | | | CCCCCTCAAGGGCATCCTGGGCTACACTG |
| | | | | | | AGCACCAGGTGGTCTCCTCTGACTTCAAC |
| | | | | | | AGCGACACCCACTCCTCCACCTTTGACGC |
| | | | | | | TG |
| chr12 | 29394350 | 29394400 | tag_20390 | + | 40 | ACTTTTTTTTTTTTTTTTTAGCAGTTTGA |
| | | | | | | GTTGGTGTAGTGTATTCTTGG |
| chr12 | 52899800 | 52899900 | tag_21160 | − | 41 | TATGAGGAGCTGCAGAGCCTGGCTGGGAA |
| | | | | | | GCACGGGGATGACCTGCGGCGCACAAAGA |
| | | | | | | CTGAGATCTCTGAGATGAACCGGAACATC |
| | | | | | | AGCCGGCTCCAGG |
| chr12 | 57846650 | 57846700 | tag_21447 | − | 42 | TGGACCCAAACTGAGGAGCCCGGAGCTGC |
| | | | | | | CGCTGGGGGATCGGGGCCGGG |
| chr12 | 107761050 | 107761100 | tag_23158 | − | 43 | AGGGCCGGGCGGGACGGGAAACGTTAGGG |
| | | | | | | CAGCGGCCCCCGGGGTGAGGG |
| chr12 | 113050900 | 113050950 | tag_23371 | + | 44 | CGTAGCCCCAGCTGAGGCAGGAGAATTGC |
| | | | | | | TTGAGCCCAGGAACTGGAGGC |
| chr12 | 120198900 | 120198950 | tag_23669 | − | 45 | GCCAGCCCAGAACACTGGTCTCGGGCCCG |
| | | | | | | AGAAGACCTCCTTTTTCCAGG |
| chr13 | 45418300 | 45418350 | tag_25246 | − | 46 | CTTGTTACAACTGAGTCCGGGTTGGAGGA |
| | | | | | | GGTGTGGGGCCGCTGCCGCCA |
| chr13 | 69167900 | 69167950 | tag_26012 | − | 47 | CCCCCACAACCGCGCCTGGCTACTTTTTG |
| | | | | | | TATTTTTAGTAGAGACAGGGT |
| chr13 | 72494150 | 72494200 | tag_26094 | − | 48 | AATGTCAAGTTAGAAAAAATGTTGTAGTT |
| | | | | | | TCATGTTGTTGGTTTGCAAAT |
| chr13 | 96427200 | 96427250 | tag_26846 | + | 49 | TGGGTCTGAATTCCAGTCTATCACTTGTT |
| | | | | | | ATGTGACCTCGGATGAATCAC |
| chr13 | 101898250 | 101898300 | tag_27053 | − | 50 | TGAAATGCTTAGGACCAAAAGTGTTTCAG |
| | | | | | | ATTTCAGATTTTTAAAATATT |
| chr14 | 37590000 | 37590050 | tag_28303 | − | 51 | TAAGCCGTGGAAAATGTTCTTGATCATTT |
| | | | | | | GCAGTTAAGGACTTTAAATAA |
| chr14 | 39432100 | 39432150 | tag_28366 | − | 52 | TTGTCAGTGCCGCAGCCCCGACCGCCGGG |
| | | | | | | AGCTCGAACCCGAGCCGGGGC |
| Chr14 | 42274950 | 42275000 | tag_28442 | − | 53 | TTGTGGGTAAGTTCTCTCTGAGATTTGGG |
| | | | | | | AGATTCATAAATTTGTGTTTT |
| chr14 | 47937850 | 47937900 | tag_28594 | − | 54 | CACTCCTGACATCATCTGATAAAAGAAAG |
| | | | | | | TCTTCAGCCGAATTTAATTTA |
| chr14 | 58244600 | 58244650 | tag_28961 | | 55 | TGAGCCGCTTGGCGTCGTGGTATCTGAGA |
| | | | | | | AGCTGCTCTATTCCTTTCCTT |
| chr14 | 63853000 | 63853050 | tag_29171 | + | 56 | CTCAGCTGCGCCCAGAGCCTTCGGCCGGA |
| | | | | | | CCTGAAAAAGCGAGAGGGAGA |
| chr14 | 67816550 | 67816600 | tag_29318 | − | 57 | TCTGTCACCTGACTCGGCTCAAACATGGC |
| | | | | | | TGCGCTGAGAGCTCTATTGCT |
| chr14 | 69486050 | 69486100 | tag_29391 | + | 58 | GTTCTGAGATTCACTACTGGTATGGCCTG |
| | | | | | | GAGAATGCAGACCCTGTCCAG |
| chr14 | 88649650 | 88649700 | tag_30094 | + | 59 | CCAAAACTATTCATTTGCATTGTAGTAGT |
| | | | | | | TTGTACAGTCATATGTAAAAC |
| Chr14 | 90396800 | 90396850 | tag_30143 | − | 60 | GTCGGGGAAGCGTTCTAGGCTGGGCGCGC |
| | | | | | | GGTCTCGGGTCAGGTGTGGCG |
| chr14 | 92486050 | 92486100 | tag_30219 | + | 61 | TATGTCCTGGTGAGGCTCCAGCAAGCAGT |
| | | | | | | GAAGCCCCACCTGTCCATCCC |
| Chr15 | 38351850 | 38351900 | tag_31664 | + | 62 | CATGTTTTGTTACAGGTTGTAGAGTATTT |
| | | | | | | GCAGAAGGAAACCATTTCTGG |
| chr15 | 40873650 | 40873700 | tag_31796 | − | 63 | GGGACACAGCGGGACAGGTGAGAAGCTGG |
| | | | | | | GCGCGGCCTCTACTGGTCTGC |
| chr15 | 62053800 | 62053850 | tag_32551 | − | 64 | TTTATCCCACTCCTGACAGTTTATCCCAC |
| | | | | | | TCTTGACACTTCCCCTTGAGC |
| chr15 | 69058600 | 69058650 | tag_32828 | − | 65 | ACACCAGGTCTCCTGGCCTTGCTGACTCC |
| | | | | | | CAGTCCAGGGAGATGGAGGCT |
| chr15 | 90294050 | 90294100 | tag_33736 | + | 66 | ATATGGTGACCTCCTGGGAGCGGGGGACC |
| | | | | | | ACCAGGTTGCCTAAGGATGGG |
| chr16 | 2762350 | 2762600 | tag_34360 | + | 67 | CACCAGCCCGGAGGGGCAGGTCTCGGTCT |
| | | | | | | AGAACACCTGCTAGGCGCAGATCTAGGAC |
| | | | | | | CCGATCACCAGTACGACGCAGGTCTCGTA |
| | | | | | | GTAGATCACCAGCCAGGAGAAGTGGCAGG |
| | | | | | | TCACGCTCTAGAACCCCAGCTAGACGTGG |
| | | | | | | CCGCTCACGCTCCAGAACCCCAGCCAGAC |
| | | | | | | GTGGCCGCTCACGCTCTAGAACCCCAGCT |
| | | | | | | AGACGCAGTGGTCGCTCACGCTCCAGAAC |
| | | | | | | ACCAGCCAGGAGAGGGAG |
| chr16 | 3305500 | 3305550 | tag_34437 | + | 68 | TGGCTCTGTACCTGGACAGGGCTGCGGTA |
| | | | | | | GGCCAGCGGTGGGCTGGCGGT |
| chr16 | 15866000 | 15866050 | tag_34894 | − | 69 | CTGCAATATCTGATTACATTGATGAATTC |
| | | | | | | CGTTGTATTGTATGTGTGAAT |
| chr16 | 16144500 | 16144550 | tag_34906 | + | 70 | ATTAGAATGAGTAGTAGTGTCTGGGTGCA |
| | | | | | | GTGACTCATGCCCCATTGAAA |
| chr16 | 24205200 | 24205250 | tag_35164 | − | 71 | TGAGCCCAGGAACTGGAGGCTGCAGTGAG |
| | | | | | | CTATGACTGTTCCACTTCACT |
| chr16 | 25107050 | 25107100 | tag_35202 | − | 72 | GTCGCAGCCACCGGTCACCTCAGCTGTGA |
| | | | | | | ACCATGTAGGAGCCGTCTGGG |
| chr16 | 30535100 | 30535150 | tag_35448 | + | 73 | CCTCGTGAACTCAGAAGTTAGTTTTCGTC |
| | | | | | | TCTGGACCTTTTTATAATCGG |
| chr16 | 48178000 | 48178050 | tag_35696 | − | 74 | CCATCATGTTCTTTATTATTGTGATTGAG |
| | | | | | | ACCTTTCTGTGGGCCAGGCAT |
| chr16 | 53503150 | 53503200 | tag_35882 | − | 75 | TGTCGGGCCGCGGCGGCGCTTGGCAGCCA |
| | | | | | | GGAGCTCTGCATTGAAGGCAC |
| chr16 | 67928900 | 67928950 | tag_36401 | + | 76 | TGCCAGGGGCACTGCCTTCTCACAGCTGG |
| | | | | | | CCTTGCCCCGTCCACCCTGTG |
| chr16 | 68237500 | 68237550 | tag_36417 | − | 77 | GTGCTACTCGCTCGAACCCGCCGGGCGAA |
| | | | | | | GGAAGCACTTCCCATGCTCTC |
| chr16 | 70780900 | 70780950 | tag_36529 | − | 78 | AGTGGACTCAGGCATCAAAGAAGTGGCCA |
| | | | | | | ACGTGTGCAACCAGAGCCTGA |
| chr16 | 78380500 | 78380550 | tag_36827 | + | 79 | ATTCAGCCGTATCAGTGAAGAGTGAAGCA |
| | | | | | | CTGCACTCTTTAAGGATAGGG |
| chr16 | 85659500 | 85659600 | tag_37112 | + | 80 | AGCACAAATATGGTGACCTCCTGGGAGCG |
| | | | | | | GGGGACCACCAGGTTGCCTAAGGAGGGGT |
| | | | | | | GAACCAGGGCAGGTCGGAAATGGAGCATG |
| | | | | | | TCAAAACTCCCGC |
| chr17 | 17591900 | 17591950 | tag_38147 | + | 81 | CCTTGGATCCGGCCTCCCGCCCAGTGCCT |
| | | | | | | TTGGTCGCCGGCTGCCGCACC |
| chr17 | 31534850 | 31534900 | tag_38643 | + | 82 | CCCTGAGCTGCCCTTGCGT7CAGTTGGAA |
| | | | | | | GCCACGCAAAATGAACTGAAC |
| chr17 | 36486650 | 36486700 | tag_38844 | + | 83 | GCGTGAGAGGCGCGCGGCGGCGCAGTGAA |
| | | | | | | CAGTCTCCTTCCACAAAACCA |
| chr17 | 38604550 | 38604600 | tag_38922 | | 84 | GCAGCCCTGGCAGTGCCTCCGGCGCTTTG |
| | | | | | | CCCTGGCCTGGTGGGAGAGGA |
| chr17 | 39723550 | 39723600 | tag_39047 | + | 85 | TGACACCTAGCGGAGCGATGCCCAACCAG |
| | | | | | | GCGCAGATGCGGATCCTGAAA |
| chr17 | 39737900 | 39738000 | tag_39057 | + | 86 | TCCCCTGGACCCGCCCCCATCTGCCCAAG |
| | | | | | | ATAATTTTAGTTTCCTTGGGCCTGGAATC |
| | | | | | | TGGACACACAGGGCTCCCCCCCGCCTCTG |
| | | | | | | ACTTCTCTGTCCG |
| chr17 | 39744000 | 39744050 | tag_39062 | + | 87 | ACATCCTGGTGGTAGAGGGGAAGGGAGGA |
| | | | | | | GGTCAGACCTGTCACTCTCCT |
| chr17 | 40054350 | 40054400 | tag_39075 | − | 88 | CCAAAATGGCGATGCCTACCACCTAGAAC |
| | | | | | | TGGATTGTGCGGTAGGCTTAA |
| chr17 | 49995200 | 49995250 | tag_39625 | − | 89 | TAACCGGCTGGCCGGGCGGAGCTGGCAGC |
| | | | | | | ATTTGATTGTGGCTTGGGACG |
| chr17 | 50187300 | 50187350 | tag_39646 | − | 90 | GAATCCTGAACAAACAATCTGATCTAGCT |
| | | | | | | TTGGCCTCTCTGCTCCCCAAT |
| chr17 | 50867150 | 50867200 | tag_39714 | − | 91 | CGCCACACTCTGGGCACGGCGTGGGCTGG |
| | | | | | | GACATTTGGAATAAACGGATC |
| chr17 | 58083400 | 58083450 | tag_39984 | + | 92 | CGCGCGTCGGCCGCGCTCAGCGGCAGAGC |
| | | | | | | GGAGCGCAGCTGTGAGGCGCC |
| chr17 | 63827000 | 63827050 | tag_40255 | | 93 | TAGTGTGCGGAGAGGGGCGCGGGGTGGAA |
| | | | | | | TTGCTGCGGGAAGTGGGAAAC |
| chr17 | 64919400 | 64919450 | tag_40314 | − | 94 | GCGGCTGAAGGCGATCCGCAGTGAGGCCC |
| | | | | | | CAGCCATTCGGATTGAGCCTT |
| chr17 | 67717950 | 67718000 | tag_40442 | + | 95 | GCAGCGCTGGAGGAAGAATTCACGTTGTC |
| | | | | | | TTCGGTAGTCCTGAGCGCCGG |
| chr17 | 68248100 | 68248150 | tag_40474 | + | 96 | GGCCTGTCGGGTCAGGGCGGTTCGCGGGT |
| | | | | | | GCTGTCAGAGCTGGGCCGGGG |
| Chr17 | 75261900 | 75261950 | tag_40741 | + | 97 | ATGGCTGCCCCCGCAGTGAAGGTTGCCCG |
| | | | | | | AGGATGGTCGGGCCTGGCGTT |
| chr17 | 79782300 | 79782350 | tag_40981 | + | 98 | GGGCAGAGGCAGTGTGGGCTGATGATGTG |
| | | | | | | CTTTGGCCTTCTCGGGACTGT |
| chr17 | 81133250 | 81133300 | tag_41079 | − | 99 | CTGGCCTGGCAGGTGACGGTGCTGGATGT |
| | | | | | | GGCCTTTTTGCCTTTTCTAAA |
| Chr18 | 750700 | 750750 | tag_41311 | − | 100 | TTTCCTTAACATTCTTCCTCCCCTGCCTG |
| | | | | | | GCCCAGTCCTTATCCCCTGCT |
| chr18 | 797950 | 798150 | tag_41315 | − | 101 | AAGAAAATAGACAATACACTTATCTGGCT |
| | | | | | | GGGGAGATACCATGATCATGAAGGTGGTT |
| | | | | | | CTCAGAGTGAGGCTCATCCATTGCACTTT |
| | | | | | | GGTTGTGCTGACCCCTGTGATTTCCCCAA |
| | | | | | | ATGCAGAATTTGTAGTAGTGAGGGACTGT |
| | | | | | | GTTCGTGCTTTCCCCTGTCATTTTTTGTC |
| | | | | | | CTAAGAGATCATAGTGTGAAGTTTAT |
| chr18 | 14830150 | 14830250 | tag_41793 | + | 102 | ACTGCCATGAAGCTCTGCAGAAGAAAGAT |
| | | | | | | CTGGAAGTGGGAGACACTTTCACTATATA |
| | | | | | | TAGTGGCTCCCACTTCCAGATCTTTCTCT |
| | | | | | | CTGTATATATAGT |
| Chr18 | 51356800 | 51356850 | tag_42903 | | 102 | CATTGGTGATTAAGGCTTCAACATACAAA |
| | | | | | | TTTCGGGGGGGACAAAAACAT |
| chr19 | 29942250 | 29942300 | tag_45203 | + | 104 | GCGGCCGCCACGCGACGCCTGGCTGGGCC |
| | | | | | | CGCACCGGAGAGGCGTCTCGG |
| chr19 | 46346900 | 46346950 | tag_45995 | − | 105 | CCCTTTCGCCACAGAGCGCGCGGAGGACA |
| | | | | | | AGGTGACCAGAGGTTCCCCAG |
| chr19 | 52962800 | 52962850 | tag_46333 | − | 106 | AGTTGCACGGCGGCGTGTGCGTTTCCTAG |
| | | | | | | TTGTCTGGTGCTGCTATATAG |
| chr19 | 53671950 | 53672000 | tag_46359 | + | 107 | TCAGACATGACATTCAGACTGAGGTCCTC |
| | | | | | | AAAACTGAGGGGCATTTTCTG |
| chr19 | 57819550 | 57819600 | tag_46611 | − | 108 | AGGCTGTAAAGAGGGCGCTACCCAGAGGT |
| | | | | | | GCATCTGCAGGAAAAGCCCAC |
| chr2 | 9615450 | 9615500 | tag_47042 | + | 109 | ATATAGGAAAGAACCACAGGTAGTTTCAT |
| | | | | | | GTGGTTGGAGGACAGAGGGAA |
| chr2 | 10302900 | 10302950 | tag_47066 | + | 110 | GGCAGGCGGGCCGCGGCGGCGGCGGGCAG |
| | | | | | | CGGACGGGCGGACTGACGGGC |
| chr2 | 69518000 | 69518050 | tag_49256 | + | 111 | AGTCCTAGCTACTTGAGAGACTCAGCTGG |
| | | | | | | GATGATCACTTGAGCCCAGGA |
| chr2 | 85539000 | 85539050 | tag_49878 | − | 112 | AGGGCAGCTCGCCCCGCGGAGTCCGGGCT |
| | | | | | | GAACCACACTGCGCGGCCGCG |
| chr2 | 152284400 | 152284450 | tag_52186 | − | 113 | AGGTAGTGAGTTATCTCAATTGATTGTTC |
| | | | | | | GCTTTCAGTTACAGATTAAGC |
| chr2 | 173395050 | 173395100 | tag_52889 | | 114 | TGCTGTTCTCCAACCAGTGCTTATGCTGC |
| | | | | | | AGGCAATTTTGTTGTTCAGAA |
| chr2 | 174594100 | 174594150 | tag_52936 | − | 115 | CTAATCAGACGTTGCTGAAGTATTGTTTT |
| | | | | | | TCAATGATGATTGACTGAGAA |
| chr2 | 218341950 | 218342000 | tag_54843 | + | 116 | CCTTCTGTCCCCTGCTCCCTTGTTCCCCA |
| | | | | | | GTCCCCTGTGTCATCAAGATG |
| chr2 | 239069350 | 239069500 | tag_55663 | − | 117 | CTCACCAAGCAAGTGTCGTGGGGCTTGCT |
| | | | | | | GGCTTGCACCGTGACTCACTCTCACTAAG |
| | | | | | | CAAGTGTCGTGGGGCTTGCTGGCTTGCAC |
| | | | | | | CGTGACTCACTCTCACTAAGCAAGTGTCG |
| | | | | | | TGGGGCTTGCTGGCTTGCACCGTGACTCC |
| | | | | | | CTCTC |
| chr20 | 9068700 | 9068750 | tag_56150 | + | 118 | CGAGGCGACAGCTCGGGCTCTGGAGCCGG |
| | | | | | | GAGGCGAGAACGAGGAGGGAG |
| chr20 | 25390750 | 25390800 | tag_56729 | | 119 | TTCCGGGCTCCGGGCTCTGGGTGGCGGCG |
| | | | | | | GCTGTGAGCGGCGGCACTGCG |
| chr20 | 35765850 | 35765900 | tag_57047 | + | 120 | GAGTCTGAGATCAGTCTGGGCCACATAGC |
| | | | | | | GAGACCCCGTCTCTATGTTAA |
| chr20 | 36870700 | 36870750 | tag_57094 | + | 121 | GTTCAAGACCAGCCTTGGCAATATAGCGA |
| | | | | | | GACCCCGTCTCTACAAAAACA |
| chr20 | 50750550 | 50750600 | tag_57742 | + | 122 | AAAAGCATATATACCTCTGACCAGTGACG |
| | | | | | | TGCAATAGGCATGACACGAGT |
| chr20 | 50752950 | 50753000 | tag_57747 | + | 123 | TTACAAATTTGACTCTTGAATGGCAAAAT |
| | | | | | | AATGTTAGTATGTAGAAGGTT |
| chr20 | 53738650 | 53738800 | tag_57859 | + | 124 | ACGACTCCTGGTTTGCCACAAGCCCGGCC |
| | | | | | | TCTGTAGTGAGAGAGCTGTGACTGCGTTT |
| | | | | | | CCAGCTCCTTGAAGGCAGAGAGACTCCTG |
| | | | | | | CCTTTCGGGGCGGGCGGGCTGGAACAAAG |
| | | | | | | ACACCAACGGGGCCACCTCGGAAAGTCTT |
| | | | | | | TTTGA |
| chr20 | 54208100 | 54208150 | tag_57876 | + | 125 | ATGGCGGCCACCATGAAGAAGGCGGTGAG |
| | | | | | | TGGGGAGCTCGGGGCTCTGGA |
| chr20 | 58448250 | 58448350 | tag_58054 | + | 126 | AACACCAAGAGCAGCTCTGAGATCATGCT |
| | | | | | | GGCCCTACGCGAATTGAGTTTCTGTGGCC |
| | | | | | | TAATTGGATTTGGAGAACGCCTTCCCTGG |
| | | | | | | CCCCTTTTCCTCA |
| chr20 | 62861750 | 62861800 | tag_58278 | − | 127 | AACGGCGCGCCGGCTGTGGCCGGCGCAGA |
| | | | | | | GTAGTGCTCGGGCCGGGGGTC |
| chr22 | 27922350 | 27922400 | tag_60225 | − | 128 | TGAGATTGGTAGACATATGACACTGGTAG |
| | | | | | | AATTAGTTTGAACATCTGTGT |
| chr22 | 33704900 | 33704950 | tag_60463 | − | 129 | TGTGGGTCCTCTGCTCTGGCAACAGGCAG |
| | | | | | | AGCACTGCTTTAGAGCCTCTG |
| chr22 | 36775050 | 36776100 | tag_60618 | − | 130 | TTGGAGAGTGCATCCGGCCCGGTACTTGT |
| | | | | | | GATCGGAGGAGAGCCGGATCG |
| chr22 | 50508250 | 50508300 | tag_61228 | + | 131 | TGGCGCCAGAACTAGTGGCGGGCTGAGGA |
| | | | | | | CGCCGTACCCCTCGGAAGGCA |
| chr3 | 47266450 | 47266500 | tag_62984 | + | 132 | CTTGGCAACATAGCGAGACCCCGTCTCTA |
| | | | | | | CAAAAAAATTTAAAAATTAGC |
| chr3 | 48159000 | 48159050 | tag_63026 | − | 133 | CCACGAGGACTTTAAAGAAGACCTGAAGA |
| | | | | | | AGTTCCGCACCAAGAGCCGGA |
| chr3 | 113263200 | 113263250 | tag_65168 | + | 134 | AGGCTGCTAAGTCATCCTGGCAGCATTGC |
| | | | | | | CACATGAGCCCCATGCGGTGC |
| chr3 | 149752400 | 149752450 | tag_66565 | − | 135 | GTGAAGATGCTGCTGGAATTGTCCGAGGA |
| | | | | | | GCATAAGGAACACCTGGCCTT |
| chr3 | 169764600 | 169764650 | tag_67280 | − | 136 | TGAGCTGTGGGACGTGCACCCAGGACTCG |
| | | | | | | GCTCACACATGCAGTTCGCTT |
| chr3 | 184298900 | 184298950 | tag_67762 | − | 137 | CACTGCTGTAGATGGGCGGTCTGCGAGCG |
| | | | | | | GAGTTACCGAGTTTTACTCCG |
| chr3 | 194159250 | 194159300 | tag_68175 | + | 138 | CCCTGGATGCCTGCCCCTTGAATGGGGGT |
| | | | | | | CAGGCTGTGCATACATTGTGA |
| chr3 | 195658050 | 195658100 | tag_68250 | + | 139 | CAGTCTGCGCAGGGACTGGCGGGACTGCG |
| | | | | | | CGGCGGCGACTACAGACGTGT |
| chr4 | 7112150 | 7112400 | tag_68635 | − | 140 | GGGATGTGAGGGCGATCTGGCTGCGACAT |
| | | | | | | CTGTCACGCCATTGATTGCCAGGGTTGAT |
| | | | | | | TCGGTTGATCTTGCTGGCTAGACGGGTGT |
| | | | | | | CCCCTTCCTCCCTCACTGCTCCACATGCG |
| | | | | | | TCCCTCCCAAAGCTGCATGCTCCATTGAC |
| | | | | | | CATCCCCAACAGAGGAGGACCGGTCTTCG |
| | | | | | | GTCAAGGGTATATGAGTAGCTGCACTCCC |
| | | | | | | CTGCTAGAACCTCCAAACAAGCTCTCAAG |
| | | | | | | GTCCAAATGACACTGGGG |
| chr4 | 28712100 | 28712150 | tag_69423 | + | 141 | GCTTAAAGGAAAGCAAAAGCTGTGTTGAG |
| | | | | | | AGAATAAAACAGGGATAAGTC |
| chr4 | 81382050 | 81382100 | tag_71049 | + | 142 | AAAATGCCTATTTAAATTACTATTTCATT |
| | | | | | | ATTTTTCTCAAAAGTGTGAAA |
| chr4 | 87505600 | 87505650 | tag_71261 | − | 143 | CCCAGGAACTGGAGGCTGCAGTGAGCTAT |
| | | | | | | GATTGCAATTACACTCCAGCC |
| chr4 | 140152300 | 140152350 | tag_72898 | + | 144 | TGGGGAGAGGAGGCGAGAGGCTCTCCTTC |
| | | | | | | CCCGCTTCCCCCCTAGGGGTT |
| chr4 | 145186850 | 145186900 | tag_73067 | + | 145 | GACTGCAGTCCTGAGACCCTATAACCTGT |
| | | | | | | ATGACTAGAGAAGTGAAACTA |
| chr4 | 151442350 | 151442400 | tag_73273 | + | 146 | GAAAAACCCCCCAAACCTTAAAAATGTAG |
| | | | | | | AATCTCTCAGCTAATCTATAT |
| chr4 | 165112750 | 165112800 | tag_73762 | − | 147 | CAGGGCTTCGGCCTCCGGCGTCGGGAAAT |
| | | | | | | GGCGGCGGGGGGCAGGATGGA |
| chr4 | 173268150 | 173268200 | tag_74031 | + | 148 | AAAAATGTTAGGGTGGTGCAAAAGTGATC |
| | | | | | | GTGGTTTTTGCAATTTTTTAA |
| chr5 | 14001200 | 14001250 | tag_75126 | − | 149 | TATTGTGTGATACTGAGGCTTGGAGTGTG |
| | | | | | | AATGAATCCTTCACCCAGGTA |
| chr5 | 173888350 | 173888400 | tag_80583 | + | 150 | TCGGCTCGGTCCTGAGGAGAAGGACTCAG |
| | | | | | | CCGCGGCTGCGGGACCCGGGC |
| Chr5 | 174724600 | 174724650 | tag_80619 | + | 151 | CGCCGCTGCCGGGTTGCCAGCGGAGTCGC |
| | | | | | | GCGTCGGGAGCTACGTAGGGC |
| chr6 | 16453150 | 16453200 | tag_81642 | | 153 | CCCCTCTATCAATGATGAGACTGATGCTG |
| | | | | | | AGAAAAGTAACATGATTATGT |
| chr6 | 24705400 | 24705450 | tag_81899 | − | 153 | TACACATTAAAGCAGATCTGGAGTCTGAA |
| | | | | | | GTAGCTATAAAGCAGCTATAA |
| chr6 | 38229900 | 38229950 | Lag_82871 | + | 154 | AGAAGCATGCCTTTCAGGGCATTGATAAA |
| | | | | | | AAGAGTGAAATGTTCAGGACC |
| chr6 | 52264600 | 52264650 | tag_83427 | | 155 | CTGAGCAAGATGCAGGATGACAATCAGGT |
| | | | | | | CATGGTGTCTGAGGGCATCAT |
| chr6 | 127266850 | 127266900 | tag_85800 | + | 156 | GCGCGTTCCCGGCAGCTGCGGGCTCCGAG |
| | | | | | | GCCAGAGAGAAAAGACTGCGA |
| chr6 | 166956500 | 166956550 | Lag_87323 | − | 157 | GAGCAACGCGACTGACCGTGGTCGTGGGC |
| | | | | | | GGACGGCGGCTGCAGCGTGGA |
| chr7 | 6059100 | 6059200 | tag_87724 | − | 158 | CTGGTTTTCCGTCTGGTGAGGGGTTACTT |
| | | | | | | CCGGGTCGGACGGCGCTAGCTGCAGCATC |
| | | | | | | GGAGTGTGGCAGTGCTGGGCTGGCCGGCG |
| | | | | | | GGCTGGGCTGGGG |
| chr7 | 6448050 | 6448100 | tag_87734 | + | 159 | CTGCGCAGAAACGCTGATCCGGAAGGCGC |
| | | | | | | TGGCTGAGTCGATTGCAGGTC |
| chr7 | 45111800 | 45111850 | tag_89181 | + | 160 | CTTTCGGCGGGTGACATCTTTGCTGAGGG |
| | | | | | | CTCAAGCGGAGCGATAGGTCA |
| chr7 | 56289400 | 56289450 | tag_89671 | + | 162 | CTTCGAAAATGAGGGTGAAGATGAAGCCA |
| | | | | | | TGTTTGTAGAATATAGAAAAC |
| chr7 | 95434950 | 95435000 | tag_90904 | | 162 | GGAGCTGCTGGCCAGGCCGGAGCGAGGCA |
| | | | | | | GCGCGCCCGGCTCCCGCGCCA |
| chr7 | 128839300 | 128839350 | tag_92163 | + | 163 | TGGCAGGTCTAGTGTCTTTGCCACTTGCC |
| | | | | | | TGGTGATTTCTATGATGAAAT |
| chr7 | 157410800 | 157410950 | tag_93308 | + | 164 | GGTTACTTCTGTGGACTTGGGCCGAACAG |
| | | | | | | CACTGTCTAAGCAGGACATGAAAAGAAGG |
| | | | | | | GGGAAGCGTCTCTCCTTTTCCCTTCATGG |
| | | | | | | AACTTTCCATTGAAAAATTAGCCCCTTCC |
| | | | | | | AGTCCTTCTCGGATGAAGCACAGTTGCCG |
| | | | | | | GTTAC |
| chr8 | 9780950 | 9781000 | tag_93694 | + | 165 | CAGCAACTGTGATACCTTG7AGAATATGA |
| | | | | | | GTGATATGCAAGCTGTGTTTT |
| chr8 | 48198250 | 48198300 | tag_95072 | − | 166 | AACCCCAGGCCAGGGAGGCCCGGGTTTGG |
| | | | | | | GATGCCTTCCTCGGGAG7GTG |
| chr8 | 60281350 | 60281400 | tag_95457 | − | 167 | AGGCCACAGCCGCTCCCTCGCTCTGCTGG |
| | | | | | | GGCCTCCGGACGCGCTTCCCA |
| chr8 | 99893500 | 99893650 | tag_96875 | − | 168 | GTTGGTGTTGAGGTGAGTCCGGTCCCTTT |
| | | | | | | TGCATCCCTACCCCGACACTGCGGGTTGT |
| | | | | | | CACAACGGCACCCTCCCGCTTTCTCTCTG |
| | | | | | | CCTCGGATTTAGTCGTGACTGTGTGTCTC |
| | | | | | | CGCCGTGGTGCAGCTTCAGGCCTCTCCCG |
| | | | | | | CATCT |
| chr8 | 100397100 | 100397150 | tag_96893 | + | 169 | TGAAGTGAGGTAGGAGGTTGA7CAAATTT |
| | | | | | | 7CTGTATAACAGGAA7A7GGA |
| chr8 | 112519350 | 112519400 | tag_97345 | + | 170 | TGAATAATAGGCTTATATGTTATAACATC |
| | | | | | | AAAATATAATTCGAGTTTGAC |
| chr8 | 118111800 | 118111850 | tag_97581 | − | 171 | TAAGTGTAACATTCAGAACCGGGTAACAT |
| | | | | | | TCGGCGACCGAACGCGGCGGT |
| chr8 | 119325150 | 119325200 | tag_97636 | + | 172 | CACAAAGTAGGGAATAT7CAAGA7TGTA7 |
| | | | | | | TAGGTTGGTGCAAAAGTGATT |
| chr8 | 122782950 | 122783000 | tag_97758 | − | 173 | CTGCTGGTTTCCAACTTTCCGCTCATCTT |
| | | | | | | CGTCTCCGCAGCCTCCTGCAA |
| chr8 | 129984200 | 129984250 | tag_98078 | + | 174 | GCAGGGACAACAG7CAGAGGGCTGCAGGG |
| | | | | | | GCCTGAAGCCAGACACGGGAC |
| chr8 | 133571800 | 133571850 | tag_98219 | − | 175 | GACCGGAGGGAGGAGGAGGAGGAAGAAGA |
| | | | | | | GCGGAGAGAGAAGGAAGAGGC |
| chr8 | 135645650 | 135645700 | tag_98308 | − | 176 | GACTTTGGACATGAAGTCCCCAGCA7CTC |
| | | | | | | TACCAGCTCCACTGAATTAAG |
| chr8 | 143858250 | 143858350 | tag_98645 | − | 177 | GCTGTCGGCCGAGCGCGCCGTCACCGGCT |
| | | | | | | ACACCGACCCCTACACCGGGCAGCAGATC |
| | | | | | | TCCCTCTTCCAGGCCATGCAGAAGGACCT |
| | | | | | | CATCGTCCGGGAG |
| chr8 | 143859850 | 143859950 | tag_98647 | − | 178 | TGCTGTCGGCCGAGCGCGCCGTCACCGGC |
| | | | | | | TACACCGACCCCTACACCGGGCAGCAGAT |
| | | | | | | CTCCCTCTTCCAGGCCATGCAGAAGGACC |
| | | | | | | TCATCGTCCGGGA |
| chr8 | 143861450 | 143861550 | tag_98649 | − | 179 | CTGTCGGCCGAGCGCGCCGTCACCGGCTA |
| | | | | | | CACCGACCCCTACACCGGGCAGCAGATCT |
| | | | | | | CCCTCTTCCAGGCCATGCAGAAGGACCTC |
| | | | | | | ATCGTCCGGGAGC |
| chr8 | 143863050 | 143863150 | tag_98651 | − | 180 | GTCGGCCGAGCGCGCCGTCACCGGCTACA |
| | | | | | | CCGACCCCTACACCGGGCAGCAGATCTCC |
| | | | | | | CTCTTCCAGGCCATGCAGAAGGACCTCAT |
| | | | | | | CGTCCGGGAGCAC |
| chr8 | 143864600 | 143864750 | tag_98653 | − | 181 | AGCTGCTGTCGGCCGAGCGCGCCGTCACC |
| | | | | | | GGCTACACCGACCCCTACACCGGGCAGCA |
| | | | | | | GATCTCCCTCTTCCAGGCCATGCAGAAGG |
| | | | | | | ACCTCATCGTCCGGGAGCACGGCATCCGC |
| | | | | | | CTGCTGGAGGCCCAGATCGCCACGGGCGG |
| | | | | | | CGTCA |
| chr8 | 143866250 | 143866350 | tag_98656 | − | 182 | TCGGCCGAGCGCGCCGTCACCGGCTACAC |
| | | | | | | CGACCCCTACACCGGGCAGCAGATCTCCC |
| | | | | | | TCTTCCAGGCCATGCAGAAGGACCTCATC |
| | | | | | | GTCCGGGAGCACG |
| chr8 | 143868300 | 143868400 | tag_98658 | − | 183 | GGAGAACCGGAAGCTGACCGTGGAGGAGG |
| | | | | | | CGTTCAAAGCAGGAATGTTCGGGAAAGAA |
| | | | | | | ACCTACGTGAAGCTGCTGTCGGCCGAGCG |
| | | | | | | CGCCGTCACCGGC |
| chr8 | 143878400 | 143878450 | tag_98664 | − | 184 | GGCGAGCGGGCAGGTGCGGCGGGTGCGGC |
| | | | | | | GGGTGCGGCGGGTGCGGCGGG |
| chr8 | 144789800 | 144789900 | tag_98753 | − | 185 | CAGAGATGCCCCTGCTGGCCGCAAAGTGG |
| | | | | | | GTCTCATTGCTGCCCGCCGGACTGGACGT |
| | | | | | | CTCCGGGGAACCAAGACTGTGCAGGAGAA |
| | | | | | | AGAGAACTAGTGC |
| chr9 | 25677700 | 25677750 | tag_99624 | + | 186 | GGGCTCCGAGTCCCGGGAGCGGAGGCCGG |
| | | | | | | AGTCGGGTTCCTGTAGAGGCT |
| chr9 | 90099550 | 90099600 | tag_101017 | − | 187 | AGACAAGCTCGGTCTGAGGTTGTTTTCCT |
| | | | | | | TTGAACTGGCTCTCTCACATT |
| chr9 | 107363050 | 107363100 | tag_101649 | + | 188 | GAGATTGAGACCAGCCTGGGCTAACATAG |
| | | | | | | CGAGACCCCGTCTCTACAAAA |
| chr9 | 110800700 | 110800750 | tag_101776 | − | 189 | ACCACGCCATAGGCCCACACATCAGACTC |
| | | | | | | TGTAGTGTAGCGGTTATAAAA |
| chr 9 | 114168900 | 114168950 | tag_101915 | + | 190 | CCTCTAAAAAACCCATTCCCACACTAGCT |
| | | | | | | CGGACTGAGGCCAAGATAACC |
| chr9 | 123110200 | 123110250 | tag_102264 | − | 191 | TGTAGTGGTCCTGCAGGAGTGGAGCCTGT |
| | | | | | | AGGACTTGCTTCTCAGCGGCT |
| chr9 | 123111550 | 123111600 | tag_102265 | − | 192 | TGTCAGGCAGTGGAGTTACTTACAGACAA |
| | | | | | | GAGCCTTGCTCAGGCCAGCCC |
| chrX | 2792950 | 2793000 | tag_103388 | − | 193 | TGGAGGCTGCAGTTAGCTATGATCACACC |
| | | | | | | ACTGCATTCCAGCCTGAGTGA |
| chrX | 10061250 | 10061300 | tag_103625 | + | 194 | CTAATTGATCACAACCAATTACAGATTTC |
| | | | | | | TTTGTTTCTTCTCCTCTCCCA |
| chrX | 16719400 | 16719450 | tag_103865 | − | 195 | CTTTTCCTCATTCGCCAGCTTTGrAGGTG |
| | | | | | | ACTGACCTAGTAGGCATGTGG |
| chrX | 47829700 | 47829750 | tag_104884 | + | 196 | CTGGGCAACATAGCGAGACCCCGTCTCTC |
| | | | | | | TCTAGTGTGTGTGTGTGTGTG |
| chrX | 74844500 | 74844550 | tag_105594 | − | 197 | CAGGGGAGTTGAGGAGGTTTACTGCAAAC |
| | | | | | | AACTGTTCTTTTTTCTTTTGG |
| chrX | 144927750 | 144927800 | tag_107740 | + | 198 | GATTCTAGAATCACAAATAAAGCCAATTA |
| | | | | | | AAATCTTTAAATTTGTTGTAC |
| chrX | 145820500 | 145820550 | tag_107766 | + | 199 | TGGCACATCTAGCAACAGAGCCAGATCAG |
| | | | | | | AACCCAGGTAAGCTCGGTCTC |
| chrX | 152393300 | 152393500 | tag_107993 | − | 200 | ACCATGGTTGTCTGAGCATGCAGCATGCT |
| | | | | | | TGTCTGCTCATACCCCATGGTTTCTGAGC |
| | | | | | | AGGAACCTTCATTGTCTACTGCTTTACAG |
| | | | | | | GGAAATAGTGTTTTATGCATCGTGTATAT |
| | | | | | | GAGTTTAGTATTTACTCATATTCTATGAC |
| | | | | | | TCTCTACTCTTAGATCACTTCTGCCTTTT |
| | | | | | | TCTGCACATTGTTTATCTGTTCCAAA |
| chrX | 155066050 | 155066100 | tag_108138 | − | 201 | AACCCAAAAAGTCACACCTGTGTCCTGTG |
| | | | | | | CGCGGGTGCTGCAGGCTTAGG |

As a third line of evidence a dataset of small RNA profiles from 10 patient-derived xenograft (PDX) models and four normal epithelial samples (unmatched) was analyzed. As shown in FIG. 1C, these oncRNAs are largely absent in normal samples; yet can be frequently detected in PDX models. By summing the expression of all 201 oncRNAs across every sample, a simple classification rule can be derived that perfectly groups the normal and PDX profiles (FIG. 2B). Together, these findings establish the existence of a large pool of oncRNAs whose expression is strongly associated with breast cancer.

### Example 3. Identification of T3p, an oncRNA Associated with Breast Cancer Progression

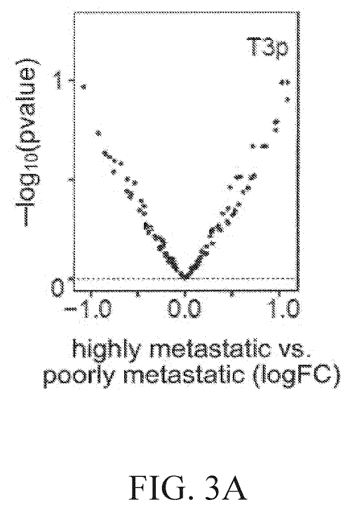

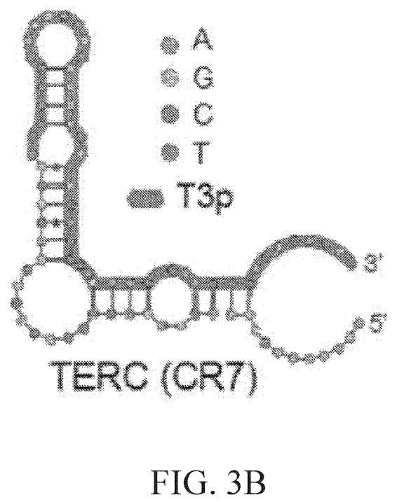

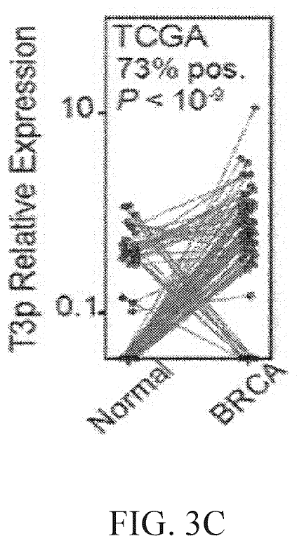

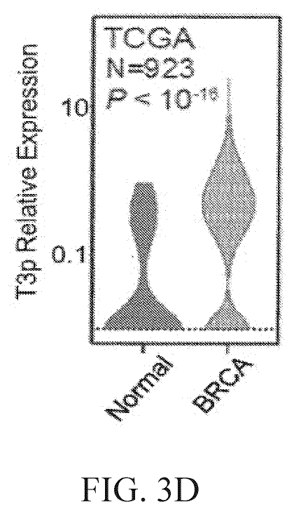

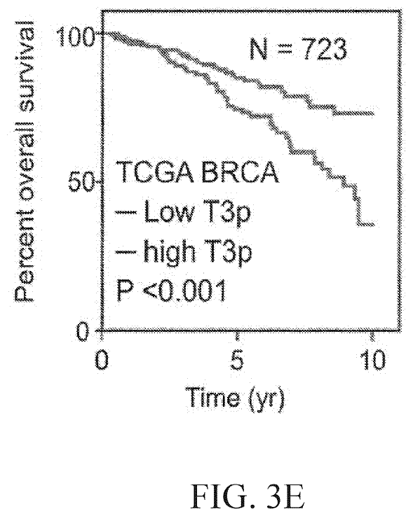

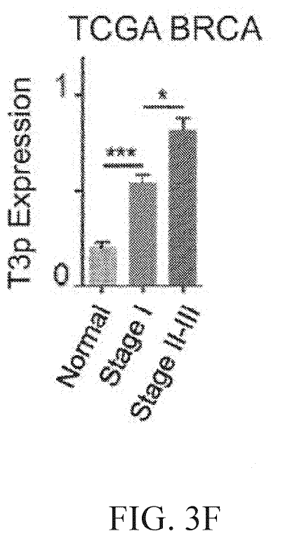

In addition to the cell lines mentioned above, highly metastatic cell lines were also profiled that had been in vivo selected in immunocompromised mice for higher metastatic capacity (1, 15). Comparing the expression of oncRNAs in these highly metastatic cells relative to their poorly metastatic parental lines, one oncRNA was noted that had significantly increased levels in highly metastatic cells (FIG. 3A). This 40-nucleotide oncRNA is generated from the 3′ end of the TERC gene, which codes for the RNA component of telomerase (FIG. 3B). As such, this previously unknown small RNA was named T3p for TERC 3′ RNA. Analysis of a previously published dataset from the same cell lines (7) further corroborated the higher expression of T3p in highly metastatic cells (FIG. 3C). This upregulation of T3p expression in metastatic cells was validated by qPCR (FIG. 3C).

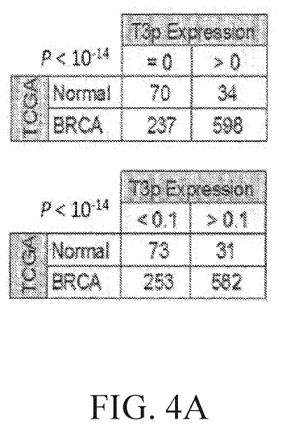

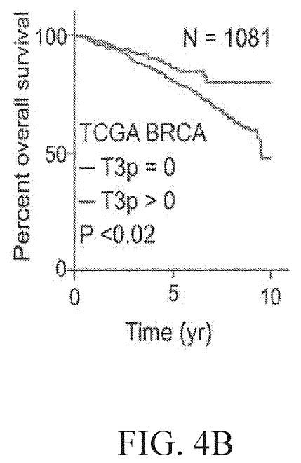

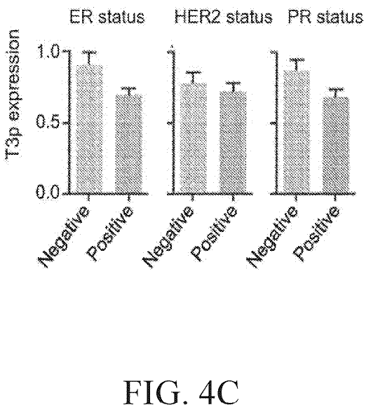

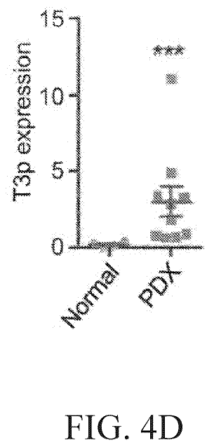

Next, whether the increased expression of T3p contributed to the pathogenesis of the underlying disease was investigated. Approximately 400 matched normal and breast cancer tumor tissue samples from TCGA-BRCA (The Cancer Genome Atlas, Breast Cancer) were analyzed, and it was noted that expression of T3p was highly cancer-specific (FIG. 3D). Then, the entire TCGA-BRCA dataset with roughly 1000 tumor samples was included. As shown in FIG. 3E, consistent with its identity as an oncRNA, T3p was not detected in the majority of normal samples, yet was detected at relatively high levels in tumor biopsies. More importantly, consistent with the higher expression of T3p in highly metastatic cell lines, a highly significant association between patient survival and T3p expression was observed (FIG. 3F). Further, as shown in FIG. 3G, expression of T3p increases across normal, stage I, and stage II or III samples in the TCGA-BRCA dataset. Detection of any level of T3p in tumor samples was strongly associated with both breast cancer and shorter overall survival (FIG. 4A and FIG. 4B). Higher expression of T3p in clinical breast cancer samples was also strongly correlated with advanced stage breast cancer (FIG. 3E). Interestingly, stratification of these cancer samples by hormone receptor and HER2 status showed no strong association of T3p levels with estrogen receptor, progesterone receptor, or HER2 receptor expression (FIG. 4C). Consistent with this finding, increased expression of T3p in PDX models of breast cancer relative to normal epithelial tissue was also noted (FIG. 4D). Together, these results establish the oncRNA T3p as a cancer-specific biomarker with robust prognostic value.

### Example 4. T3p Acts as a Broad Regulator of Gene Expression in Breast Cancer Cells

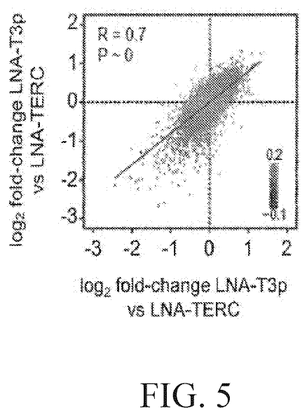

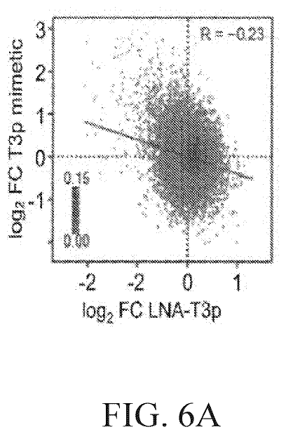

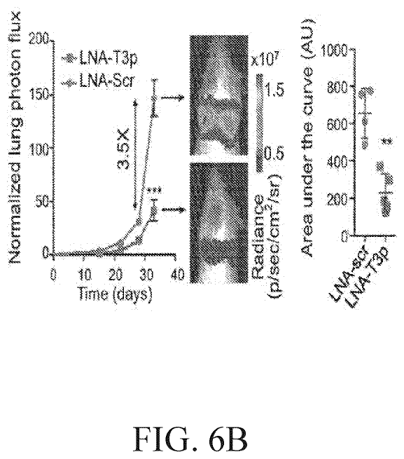

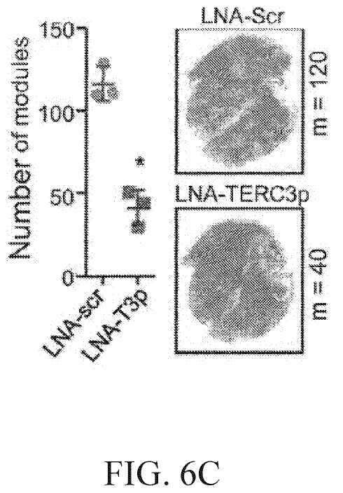

The strong associations between T3p expression and breast cancer progression from multiple independent datasets raise the possibility that T3p plays a direct and functional role in breast cancer progression. To elucidate its molecular function, it was investigated if modulating T3p expression levels resulted in regulatory consequences. To this end, T3p was silenced by transfecting highly metastatic MDA-LM2 cells with antisense locked nucleic acids (LNA) targeting T3p, or with control scrambled LNAs. Gene expression profiling was then performed to measure the genome wide regulatory impact of T3p silencing. Surprisingly, a highly significant change in the gene expression landscape of the cell upon T3p silencing, affecting thousands of genes, was observed. This is on par with the impact of many well-established post-transcriptional regulators such as small non-coding RNAs (7, 16). However, the full length TERC transcript remains a potential confounding factor, as the T3p-targeting LNA also impacts TERC function, which in turn could be responsible for the observed gene expression changes. To distinguish between these two possibilities, two independent approaches were used. First, in addition to the scrambled LNA, an anti-sense LNA against full length TERC, 5′ of T3p was also used. As shown in FIG. 5, gene expression changes induced by the anti-T3p LNA are similar regardless of whether the scrambled or anti-full length TERC LNA is used as the reference. This observation indicates that inhibition of full length TERC does not induce the same dramatic regulatory changes generated by T3p inhibition. To further strengthen these findings, a gain-of-function experiment was also performed. Using synthetic oligonucleotide as a T3p mimetic, the parental MDA-MB-231 breast cancer cells were transfected with scrambled oligonucleotide as control and then gene expression profiling was performed. Similar to the LNA experiment, a significant change in the gene expression landscape of the cell was observed. Importantly, these gene expression changes were generally anti-correlated with those observed in the loss-of-function LNA experiment (FIG. 6A). This is consistent with the expectation that anti-T3p LNAs and T3p mimetics should elicit opposite gene expression changes. Together, these observations establish T3p as a broad regulator of gene expression in breast cancer cells.

### Example 5. T3p Promotes Breast Cancer Metastasis

Given the broad regulatory effect on gene expression of T3p, as well as its association with metastasis and with poor survival in breast cancer, it was next tested whether this oncRNA could affect metastasis in vivo. To test this hypothesis, highly metastatic MDA-LM2 cells were transfected with anti-T3p LNAs and metastatic lung colonization assays were carried out by injecting these cells into the venous circulation of immunocompromised mice. In vivo imaging was then used to measure the impact of T3p inhibition on metastatic lung colonization of these cells over time. As shown in FIG. 6B, cells transfected with anti-T3p LNAs had significantly diminished lung colonization capacity. Gross histology of lungs from each cohort also revealed a significantly lower number of visible metastatic nodules in the lungs of mice injected with T3p-LNA transfected cells. As shown in FIG. 6C, visible metastatic nodules were counted in three mice from each cohort. Also shown are H&E stained representative lung sections from each cohort along with the median counts. These observations strongly support a functional role for T3p, a previously unknown ncRNA, in driving breast cancer metastasis.

### Example 6. Specific oncRNAs are Sorted into the Exosomal Compartment

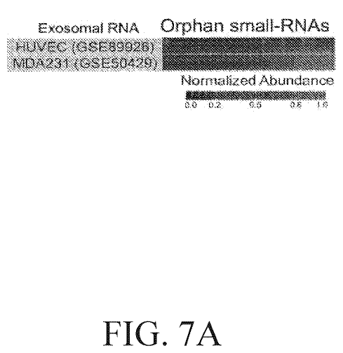

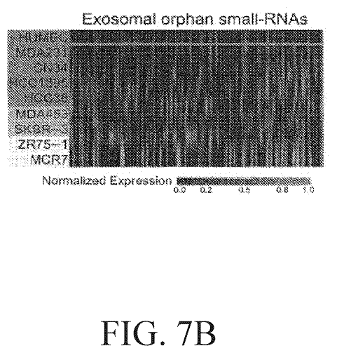

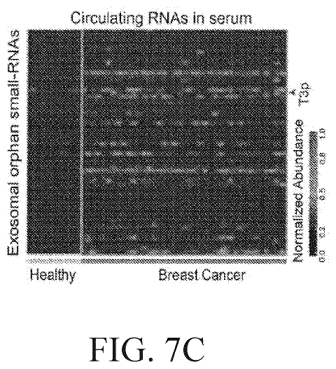

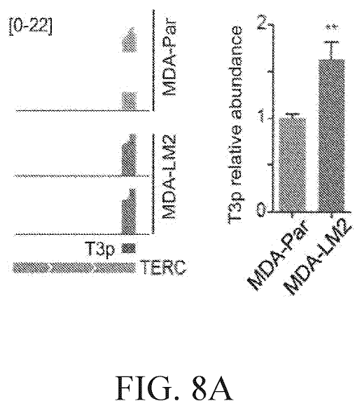

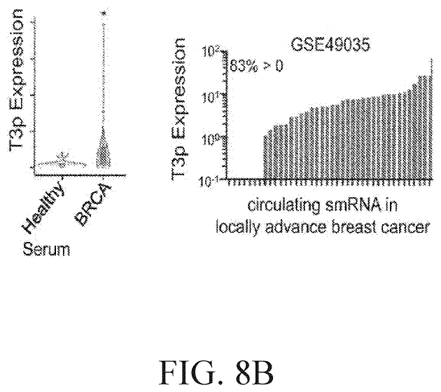

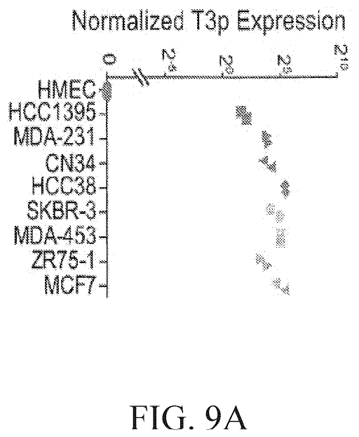

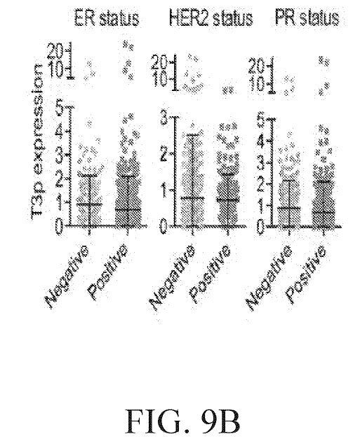

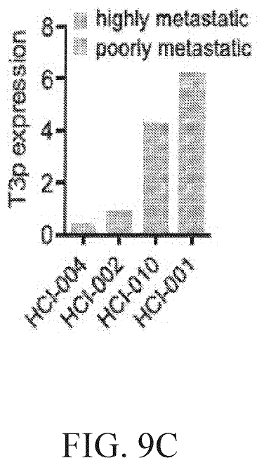

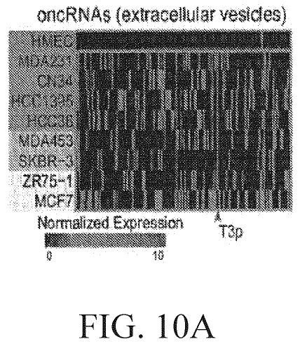

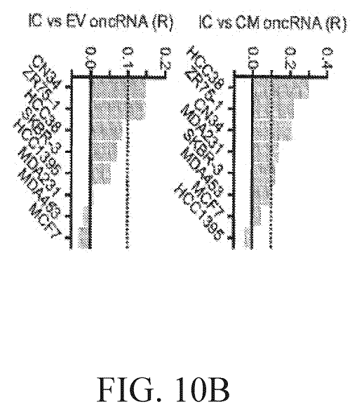

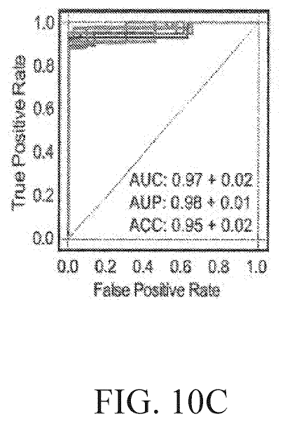

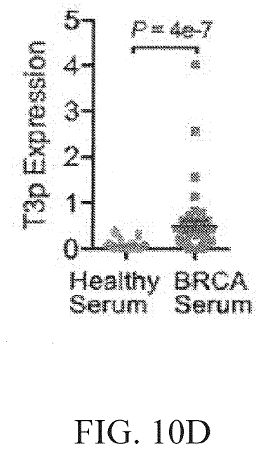

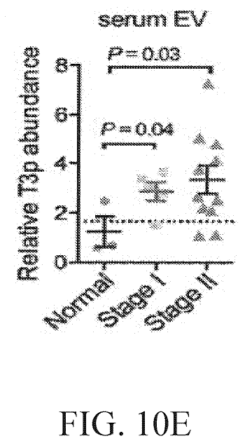

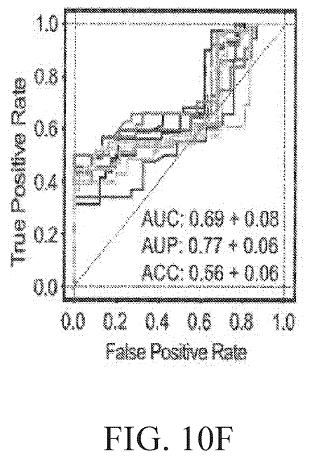

The exosomal compartment has been previously reported as a biologically relevant destination for small RNAs, such as small non-coding RNAs and tRNA fragments (28). Analysis of publicly available exosomal small RNA-seq data from MDA-MB-231 cells (29) revealed that a large number of annotated oncRNAs from this study can be detected in exosomes secreted from cancer cells (FIG. 7A). In comparison, only a handful of these oncRNAs were detected in exosomal samples from HUVEC cells (30). T3p, for example, was present in exosomes collected from MDA-MB-231 cells but not HUVEC cells (FIG. 8A). These observations prompted the next set of experiments aimed at profiling exosomal small RNAs. To this end, small RNA were isolated from exosomes secreted from eight breast cancer cell lines as well as from HMECs. Small RNA sequencing of this material revealed that of the 201 annotated oncRNAs, close to two thirds were detected in exosomal RNA from one or more of these breast cancer lines but not in HMECs (FIG. 7B). Interestingly, T3p was detected in 5 out of 8 cell lines.

To assess whether oncRNAs are also present in the circulating RNA population, a collection of RNA-seq data generated from RNA isolated from sera from breast cancer patients was re-analyzed (31). As a point of reference, data collected from sera of 11 healthy individuals was included (32). As shown in FIG. 7C, a large fraction of oncRNAs could be detected in circulating RNA samples from breast cancer patients but were generally absent from healthy individuals. This observation raises the possibility that circulating oncRNAs can be used for cancer fingerprinting from liquid biopsies. To assess this possibility, a linear model was trained on the exosomal oncRNA dataset collected from cell lines (FIG. 7B) and used it to predict the classification of circulating RNA profiles (FIG. 7C). The trained model successfully assigned 11/11 healthy samples and 31/40 samples from cancer patients (AUC: 0.96, AUPRC: 0.99, and ACC: 0.82). For example, T3p alone showed markedly different expression levels between breast cancer patients and healthy volunteers (FIG. 7C and FIG. 8B). Given the success of this simple classifier, a more generalizable machine learning approach was tested by training a Gradient Boosted Classifier on the 201 oncRNAs in the TCGA-BRCA dataset. This model, which was trained on the TCGA data, was tested on the circulating small RNA profiles in FIG. 7C. This classifier successfully classified 11/11 healthy and 37/40 patient samples (AUC: 0.976, AUPRC: 0.993, and ACC: 0.948). Based on these results, we surmise that detection of circulating oncRNAs can be used as a robust readout for the presence of an underlying cancer with high specificity. A list of 67 circulating oncRNAs that were identified is shown in Table 2, below.

TABLE 2

| | Start | End | | | |
|---|---|---|---|---|---|
| chromo- | on | on | | chromo- | |
| some | chromo- | chromo- | OncRNA | some | |
| Location | some | some | ID | strand | Nucleotide Sequence |
| chr1 | 20787250 | 20787300 | tag_860 | − | GGCGGCTGGGACTGGAGGACAGCGGTGGCGGAGGC |
| | | | | | GACTAGCGGCGGCGG |
| chr1 | 28914600 | 28914650 | tag_1217 | + | GCTCGCTCGCTCCCTCCCTCCGCTGGTGCGTTTAG |
| | | | | | TCAGTCAGCCAGCAG |
| chr1 | 149888200 | 149888250 | tag_4908 | + | TCAAACAAAATACTGGTTTTGTTTTACTGAGAGGC |
| | | | | | ACTGTGGGTTTTTGT |
| chr1 | 151372650 | 151372700 | tag_5001 | − | AGCGGCCTCTGACACCAGCACAGCAAACCCGCCGG |
| | | | | | GATCAAAGTGTACCA |
| chr1 | 154975050 | 154975100 | tag_5206 | + | TACTGACCACCCTCACCCATGAGGCTTTCTGGATC |
| | | | | | ATTCTCTGAACTTTA |
| chr1 | 162157100 | 162157150 | tag_5638 | + | ATGGGGCTGGCTGGTTGTGGGATCTGGAGGCATCT |
| | | | | | GGGGTTGGAATGTGA |
| chr1 | 162497850 | 162497900 | tag_5657 | + | GGCTGCTTGAAGTCCCGGGAGTCGGTGAGGCGGCT |
| | | | | | GCAGGTCCCTCCCTG |
| chr1 | 164559150 | 164559200 | tag_5733 | + | CTGTATATGTTTCTGGAGTCCTGAGCCTGAGCTAA |
| | | | | | ACAAAAGCAGGAGGC |
| chr1 | 167935950 | 167936000 | tag_5869 | − | GGTTCACAGCGGGCAGGGAAAGCCGCGGGAAGGGT |
| | | | | | ACTCCAGGCGAGAGG |
| chr1 | 174159550 | 174159600 | tag_6119 | + | TGGCGGAGCGAACGGGACCGGCCCGGCTTCAGAGC |
| | | | | | GCGAGGTGGAGGGTG |
| chr1 | 222618100 | 222618150 | tag_7990 | + | TGACCACAACATGGCTGCGGCGCCTGGGCTGCTCG |
| | | | | | TCTGGCTGCTCGTGC |
| chr1 | 228458100 | 228458150 | tag_8247 | + | GCGTTTCTGTTTGGAGAGACTCAGCCATCATGCCA |
| | | | | | GACCCGTCCAAATCG |
| chr1 | 245513450 | 245513500 | tag_8967 | − | CTGCCCTTATCAGTTTTGACCTGCTAGGTGCTTCA |
| | | | | | CAGAACTTTGCTTGA |
| chr10 | 47570550 | 47570600 | tag_10732 | + | GTGGGAGGATCACTTGAGCCCGGAAGTTCAAGACC |
| | | | | | ACCCTAGGCAATATA |
| chr10 | 89306850 | 89306900 | tag_12154 | + | ATGAATCTAAGAGAGAATGGAATGTATGGGAAAAG |
| | | | | | AAAGTTACTGGAACT |
| chr11 | 70203300 | 70203350 | tag_16694 | + | TGTCGATTTCCTGTAGTGAATCAGGCACCGGAGTG |
| | | | | | CAGGTTCGGGGGTGG |
| chr12 | 6537600 | 6537950 | tag_19454 | + | CACTGCCACCCAGAAGACTGTGGATGGCCCCTCCG |
| | | | | | GGAAACTGTGGCGTGATGGCCGCGGGGCTCTCCAG |
| | | | | | AACATCATCCCTGCCTCTACTGGCGCTGCCAAGGC |
| | | | | | TGTGGGCAAGGTCATCCCTGAGCTGAACGGGAAGC |
| | | | | | TCACTGGCATGGCCTTCCGTGTCCCCACTGCCAAC |
| | | | | | GTGTCAGTGGTGGACCTGACCTGCCGTCTAGAAAA |
| | | | | | ACCTGCCAAATATGATGACATCAAGAAGGTGGTGA |
| | | | | | AGCAGGCGTCGGAGGGCCCCCTCAAGGGCATCCTG |
| | | | | | GGCTACACTGAGCACCAGGTGGTCTCCTCTGACTT |
| | | | | | CAACAGCGACACCCACTCCTCCACCTTTGACGCTG |
| chr12 | 29394350 | 29394400 | tag_20390 | + | ACTTTTTTTTTTTTTTTTTAGCAGTTTGAGTTGGT |
| | | | | | GTAGTGTATTCTTGG |
| chr12 | 52899800 | 52899900 | tag_21160 | − | TATGAGGAGCTGCAGAGCCTGGCTGGGAAGCACGG |
| | | | | | GGATGACCTGCGGCGCACAAAGACTGAGATCTCTG |
| | | | | | AGATGAACCGGAACATCAGCCGGCTCCAGG |
| chr12 | 57846650 | 57846700 | tag_21447 | − | TGGACCCAAACTGAGGAGCCCGGAGCTGCCGCTGG |
| | | | | | GGGATCGGGGCCGGG |
| chr12 | 107761050 | 107761100 | tag_23158 | − | AGGGCCGGGCGGGACGGGAAACGTTAGGGCAGCGG |
| | | | | | CCCCCGGGGTGAGGG |
| chr12 | 120198900 | 120198950 | tag_23669 | − | GCCAGCCCAGAACACTGGTCTCGGGCCCGAGAAGA |
| | | | | | CCTCCTTTTTCCAGG |
| chr13 | 45418300 | 45418350 | tag_25246 | − | CTTGTTACAACTGAGTCCGGGTTGGAGGAGGTGTG |
| | | | | | GGGCCGCTGCCGCCA |
| chr13 | 69167900 | 69167950 | tag_26012 | − | CCCCCACAACCGCGCCTGGCTACTTTTTGTATTTT |
| | | | | | TAGTAGAGACAGGGT |
| chr14 | 47937850 | 47937900 | tag_28594 | − | CACTCCTGACATCATCTGATAAAAGAAAGTCTTCA |
| | | | | | GCCGAATTTAATTTA |
| chr14 | 88649650 | 88649700 | tag_30094 | + | CCAAAACTATTCATTTGCATTGTAGTAGTTTGTAC |
| | | | | | AGTCATATGTAAAAC |
| chr14 | 90396800 | 90396850 | tag_30143 | − | GTCGGGGAAGCGTTCTAGGCTGGGCGCGCGGTCTC |
| | | | | | GGGTCAGGTGTGGCG |
| Chrl5 | 62053800 | 62053850 | tag_32551 | − | TTTATCCCACTCCTGACAGTTTATCCCACTCTTGA |
| | | | | | CACTTCCCCTTGAGC |
| chr15 | 69058600 | 69058650 | tag_32828 | − | ACACCAGGTCTCCTGGCCTTGCTGACTCCCAGTCC |
| | | | | | AGGGAGATGGAGGCT |
| chr16 | 2762350 | 2762600 | tag_34360 | + | CACCAGCCCGGAGGGGCAGGTCTCGGTCTAGAACA |
| | | | | | CCTGCTAGGCGCAGATCTAGGACCCGATCACCAGT |
| | | | | | ACGACGCAGGTCTCGTAGTAGATCACCAGCCAGGA |
| | | | | | GAAGTGGCAGGTCACGCTCTAGAACCCCAGCTAGA |
| | | | | | CGTGGCCGCTCACGCTCCAGAACCCCAGCCACACC |
| | | | | | TGGCCGCTCACGCTCTAGAACCCCAGCTAGACGCA |
| | | | | | GTGGTCGCTCACGCTCCAGAACACCAGCCAGGAGA |
| | | | | | GGGAG |
| chr16 | 16144500 | 16144550 | tag_34906 | + | ATTAGAATGAGTAGTAGTGTCTGGGTGCAGTGACT |
| | | | | | CATGCCCCATTGAAA |
| chr16 | 67928900 | 67928950 | tag_36401 | + | TGCCAGGGGCACTGCCTTCTCACAGCTGGCCTTGC |
| | | | | | CCCGTCCACCCTGTG |
| chr16 | 78380500 | 78380550 | tag_36827 | + | ATTCAGCCGTATCAGTGAAGAGTGAAGCACTGCAC |
| | | | | | TCTTTAAGGATAGGG |
| chr17 | 31534850 | 31534900 | tag_38643 | + | CCCTGAGCTGCCCTTGCGTTCAGTTGGAAGCCACG |
| | | | | | CAAAATGAACTGAAC |
| chr17 | 36486650 | 36486700 | tag_38844 | + | GCGTGAGAGGCGCGCGGCGGCGCAGTGAACAGTCT |
| | | | | | CCTTCCACAAAACCA |
| chr17 | 38604550 | 38604600 | tag_38922 | | GCAGCCCTGGCAGTGCCTCCGGCGCTTTGCCCTGG |
| | | | | | CCTGGTGGGAGAGGA |
| chr17 | 58083400 | 58083450 | tag_39984 | + | CGCGCGTCGGCCGCGCTCAGCGGCAGAGCGGAGCG |
| | | | | | GAGCTGTGAGGCGCC |
| chr17 | 64919400 | 64919450 | tag_40314 | − | GCGGCTGAAGGCGATCCGCAGTGAGGCCCCAGCCA |
| | | | | | TTCGGATTGAGCCTT |
| chr17 | 81133250 | 81133300 | tag_41079 | − | CTGGCCTGGCAGGTGACGGTGCTGGATGTGGCCTT |
| | | | | | TTTGCCTTTTCTAAA |
| chr18 | 797950 | 798150 | tag_41315 | − | AAGAAAATAGACAATACACTTATCTGGCTGGGGAG |
| | | | | | ATACCATGATCATGAAGGTGGTTCTCAGAGTGAGG |
| | | | | | CTCATCCATTGCACTTTGGTTGTGCTGACCCCTGT |
| | | | | | GATTTCCCCAAATGCAGAATTTGTAGTAGTGAGGG |
| | | | | | ACTGTGTTCGTGCTTTCCCCTGTCATTTTTTGTCC |
| | | | | | TAAGAGATCATAGTGTGAAGTTTAT |
| chr19 | 29942250 | 29942300 | tag_45203 | + | GCGGCCGCCACGCGACGCCTGGCTGGGCCCGCACC |
| | | | | | GGAGAGGCGTCTCGG |
| chr19 | 53671950 | 53672000 | tag_46359 | + | TCAGACATGACATTCAGACTGAGGTCCTCAAAACT |
| | | | | | GAGGGGCATTTTCTG |
| chr2 | 10302900 | 10302950 | tag_47066 | + | GGCAGGCGGGCCGCGGCGGCGGCGGGCAGCGGACG |
| | | | | | GGCGGACTGACGGGC |
| chr20 | 9068700 | 9068750 | tag_56150 | + | CGAGGCGACAGCTCGGGCTCTGGAGCCGGGAGGCG |
| | | | | | AGAACGAGGAGGGAG |
| chr20 | 25390750 | 25390800 | tag_56729 | − | TTCCGGGCTCCGGGCTCTGGGTGGCGGCGGCTGTG |
| | | | | | AGCGGCGGCACTGCG |
| chr20 | 35765850 | 35765900 | tag_57047 | + | CAGTCTGAGATCAGTCTGGGCCACATAGCGACACC |
| | | | | | CCGTCTCTATGTTAA |
| chr20 | 36870700 | 36870750 | Tag_57094 | + | GTTCAAGACCAGCCTTGGCAATATAGCGAGACCCC |
| | | | | | GTCTCTACAAAAACA |
| chr20 | 58448250 | 58448350 | tag_58054 | + | AACACCAAGAGCAGCTCTGAGATCATGCTGGCCCT |
| | | | | | ACGCGAATTGAGTTTCTGTGGCCTAATTGGATTTG |
| | | | | | GAGAACGCCTTCCCTGGCCCCTTTTCCTCA |
| chr3 | 47266450 | 47266500 | tag_62984 | + | CTTGGCAACATAGCGAGACCCCGTCTCTACAAAAA |
| | | | | | AATTTAAAAATTAGC |
| chr3 | 169764600 | 169764650 | tag_67280 | − | TGAGCTGTGGGACGTGCACCCAGGACTCGGCTCAC |
| | | | | | ACATGCAGTTCGCTT |
| chr3 | 195658050 | 195658100 | tag_68250 | + | CAGTCTGCGCAGGGACTGGCGGGACTGCGCGGCGG |
| | | | | | CGACTACAGACGTGT |
| chr4 | 81382050 | 81382100 | tag_71049 | + | AAAATGCCTATTTAAATTACTATTTCATTATTTTT |
| | | | | | CTCAAAAGTGTGAAA |
| chr4 | 140152300 | 140152350 | tag_72898 | + | TGGGGAGAGGAGGCGAGAGGCTCTCCTTCCCCGCT |
| | | | | | TCCCCCCTAGGGGTT |
| chr4 | 145186850 | 145186900 | tag_73067 | + | GACTGCAGTCCTGAGACCCTATAACCTGTATGACT |
| | | | | | AGAGAAGTGAAACTA |
| chr4 | 173268150 | 173268200 | tag_74031 | + | AAAAATGTTAGGGTGGTGCAAAAGTGATCGTGGTT |
| | | | | | TTTGCAATTTTTTAA |
| chr5 | 14001200 | 14001250 | tag_75126 | − | TATTGTGGGATACTGAGGCTTGGAGTGTGAATGAA |
| | | | | | TCCTTCACCCAGGTA |
| chr5 | 173888350 | 173888400 | tag_80583 | + | TCGGCTCGGTCCTGAGGAGAAGGACTCAGCCGCGG |
| | | | | | CTGCGGGACCCGGGC |
| chr6 | 127266850 | 127266900 | tag_85800 | + | GCGCGTTCCCGGCAGCTGCGGGCTCCGAGGCCAGA |
| | | | | | GAGAAAAGACTGCGA |
| Chr6 | 166956500 | 166956550 | tag_87323 | − | GAGCAACGCGACTGACCGTGGTCGTGGGCGGACGG |
| | | | | | CGGCTGCAGCGTGGA |
| chr7 | 6059100 | 6059200 | tag_87724 | − | CTGGTTTTCCGTCTGGTGAGGGGTTACTTCCGGGT |
| | | | | | CGGACGGCGCTAGCTGCAGCATCGGAGTGTGGCAG |
| | | | | | TGCTGGGCTGGCCGGCGGGCTGGGCTGCGG |
| chr7 | 157410800 | 157410950 | tag_93308 | + | GGTTACTTCTGTGGACTTGGGCCGAACAGCACTGT |
| | | | | | CTAAGCAGGACATGAAAAGAAGGGGGAAGCGTCTC |
| | | | | | TCCTTTTCCCTTCATGGAACTTTCCATTGAAAAAT |
| | | | | | TAGCCCCTTCCAGTCCTTCTCGGATGAAGCACAGT |
| | | | | | TGCCGGTTAC |
| chr8 | 143859850 | 143859950 | tag_98647 | − | TGCTGTCGGCCGAGCGCGCCGTCACCGGCTACACC |
| | | | | | GACCCCTACACCGGGCAGCAGATCTCCCTCTTCCA |
| | | | | | GGCCATGCAGAAGGACCTCATCGTCCGGGA |
| chr8 | 143868300 | 143868400 | tag_98658 | | GGAGAACCGGAAGCTGACCGTGGAGGAGGCGTTCA |
| | | | | | AAGCAGGAATGTTCGGGAAAGAAACCTACGTGAAG |
| | | | | | CTGCTGTCGGCCGAGCGCGCCGTCACCGGC |
| chr8 | 144789800 | 144789900 | tag_98753 | | CAGAGATGCCCCTGCTGGCCGCAAAGTGGGTCTCA |
| | | | | | TTGCTGCCCGCCGGACTGGACGTCTCCGGGGAACC |
| | | | | | AAGACTGTGCAGGAGAAAGAGAACTAGTGC |
| chr9 | 123110200 | 123110250 | tag_102264 | | TGTAGTGGTCCTGCAGGAGTGGAGCCTGTAGGACT |
| | | | | | TGCTTCTCAGCGGCT |
| chr9 | 123111550 | 123111600 | tag_102265 | | TGTCAGGCAGTGGAGTTACTTACAGACAAGAGCCT |
| | | | | | TGCTCAGGCCAGCCC |
| chrX | 152393300 | 152393500 | tag_107993 | | ACCATGGTTGTCTGAGCATGCAGCATGCTTGTCTG |
| | | | | | CTCATACCCCArGGTTTCTGAGCAGGAACCTTCAT |
| | | | | | TGTCTACTGCTTTACAGGGAAATAGTGTTTTATGC |
| | | | | | ATCGTGTATATGAGTTTAGTATTTACTCATATTCT |
| | | | | | ATGACTCTCTACTCTTAGATCACTTCTGCCTTTTT |
| | | | | | CTGCACATTGTTTATCTGTTCCAAA |

Finally, from the 201 oncRNAs identified, the following oncRNAs shown in Table 3 were found to be the strongest performers in predicting the presence of breast cancer in a subject through analysis of serum samples.

TABLE 3

| chromo- | | | | | | |
|---|---|---|---|---|---|---|
| some | Start on | End on | | chromo- | | |
| Loca- | chromo- | chromo- | OncRNA | some | | Sequence |
| tion | some | some | ID | strand | Nucleotide Sequence | Identifier |
| chr1 | 20787250 | 20787300 | tag_860 | − | GGCGGCTGGGACTGGAGGAC | SEQ ID |
| | | | | | AGCGGTGGCGGAGGCGACTA | NO: 3 |
| | | | | | GCGGCGGCGG | |
| chr1 | 174159550 | 174159600 | tag_6119 | + | TGGCGGAGCGAACGGGACCG | SEQ ID |
| | | | | | GCCCGGCTTCAGAGCGCGAG | NO: 19 |
| | | | | | GTGGAGGGTG | |
| chr10 | 89306850 | 89306900 | tag_12154 | + | ATGAATCTAAGAGAGAATGG | SEQ ID |
| | | | | | AATGTATGGGAAAAGAAAGT | NO: 32 |
| | | | | | TACTGGAACT | |
| chr12 | 29394350 | 29394400 | tag_20390 | + | AGTTTGAGTTGGTGTAGTGT | SEQ ID |
| | | | | | ATTCTTGG | NO: 40 |
| chr12 | 52899800 | 52899900 | tag_21160 | − | TATGAGGAGCTGCAGAGCCT | SEQ ID |
| | | | | | GGCTGGGAAGCACGGGGATG | NO: 41 |
| | | | | | ACCTGCGGCGCACAAAGACT | |
| | | | | | GAGATCTCTGAGATGAACCG | |
| | | | | | GAACATCAGCCGGCTCCAGG | |
| chr16 | 78380500 | 78380550 | tag_36827 | + | ATTCAGCCGTATCAGTGAAG | SEQ ID |
| | | | | | AGTGAAGCACTGCACTCTTT | NO: 79 |
| | | | | | AAGGATAGGG | |
| chr17 | 31534850 | 31534900 | tag_38643 | + | CCCTGAGCTGCCCTTGCGTTC | SEQ ID |
| | | | | | AGTTGGAAGCCACGCAAAAT | NO: 82 |
| | | | | | GAACTGAAC | |
| chr17 | 36486650 | 36486700 | tag_38844 | + | GCGTGAGAGGCGCGCGGCGG | SEQ ID |
| | | | | | CGCAGTGAACAGTCTCCTTC | NO: 83 |
| | | | | | CACAAAACCA | |
| chr20 | 58448250 | 58448350 | tag_58054 | + | AACACCAAGAGCAGCTCTGA | SEQ ID |
| | | | | | GATCATGCTGGCCCTACGCG | NO: 126 |
| | | | | | AATTGAGTTTCTGTGGCCTA | |
| | | | | | ATTGGATTTGGAGAACGCCT | |
| | | | | | TCCCTGGCCCCTTTTCCTCA | |
| chr4 | 173268150 | 173268200 | tag_74031 | + | AAAAATGTTAGGGTGGTGCA | SEQ ID |
| | | | | | AAAGTGATCGTGGTTTTTGC | NO: 148 |
| | | | | | AATTTTTTAA | |
| chr9 | 123110200 | 123110250 | tag_102264 | − | TGTAGTGGTCCTGCAGGAGT | SEQ ID |
| | | | | | GGAGCCTGTAGGACTTGCTT | NO: 191 |
| | | | | | CTCAGCGGCT | |

The current hypothesis of breast cancer development and progression emphasizes the transformation of aberrant cell machinery leading to increased selection for oncogenic phenotypes. Consequently, the development of cancer therapy and diagnostics aims to target these pathways to reduce the ability of these cancer cells to survive, divide, or spread. In the present examples, it was proposed that cancer cells may also evolve to create cancer-specific regulatory pathways. Through a systematic and unbiased discovery step across eight breast cancer cell lines and HUMECs combined with clinical breast cancer data, a population of 201 RNA species that are expressed in breast cancer cells, but are largely undetectable in normal tissue, have been identified. These RNA molecules, which have collectively been named orphan non-coding RNAs, provide a pool of novel potential regulators that cancer cells can utilize to engineer new regulatory circuits. Poorly and highly metastatic cells were compared to ask whether oncRNAs can function in breast cancer progression. It was discovered that one of these RNAs, which was termed T3p, is strongly associated with metastatic progression in both cell line models and clinical datasets. Finally, the examples described herein show that oncRNAs can be detected in circulating and exosomal compartments.

These findings offer a new paradigm for cancer progression and how tumors can evolve and rewire regulatory pathways en route to metastatic spread. Moreover, these results also suggest a novel avenue for breast cancer detection and monitoring that could complement current methods. Current screening methods for breast cancer, including mammography and ultrasounds, offer limited detection signals due to low resolution, and are biased given their reliance on user interpretation. Other strategies in development for detecting early cancer have focused on “liquid biopsies,” which attempt to detect cancer biologic markers, including circulating tumor cells and DNA, from a patient's serum. The higher abundance of secreted exosomes within patient serum and the cancer cell specificity of oncRNA may provide a potent addition to our repertoire for a more reliable method of early detection or screening. In other words, the work described herein supports the notion that oncRNAs act as a digital fingerprint—i.e., each marker is detected or not detected—for the underlying tumors.

Although the examples described herein have largely focused on the role of T3p in breast cancer metastasis, the approaches and concepts presented here are generalizable and can be applied across several cancers. Taken together, these findings open the possibility that further examination of the cancer-specific RNA landscape and investigation into oncRNAs may yield alternative therapeutic and diagnostic methods across many cancer types.

### REFERENCES

1. S. F. Tavazoie et al., Endogenous human microRNAs that suppress breast cancer metastasis. Nature. 451, 147-U3 (2008).
2. C. J. David, M. Chen, M. Assanah, P. Canoll, J. L. Manley, HnRNP proteins controlled by c-Myc deregulate pyruvate kinase mRNA splicing in cancer. Nature. 463, 364-368 (2010).
3. S. Vanharanta et al., Loss of the multifunctional RNA-binding protein RBM47 as a source of selectable metastatic traits in breast cancer. eLife. 3 (2014), doi: 10.7554/eLife.02734.
4. L. Fish et al., Muscleblind-like 1 suppresses breast cancer metastatic colonization and stabilizes metastasis suppressor transcripts. Genes Dev. 30, 386-398 (2016).
5. L.-Y. Chen, J. Lingner, AUF1/HnRNP D RNA binding protein functions in telomere maintenance. Mol. Cell. 47, 1-2 (2012).
6. H. Goodarzi et al., Modulated expression of specific tRNAs drives gene expression and cancer progression. Cell. 165, 1416-1427 (2016).
7. H. Goodarzi et al., Endogenous tRNA-Derived Fragments Suppress Breast Cancer Progression via YBX1 Displacement. Cell. 161, 790-802 (2015).
8. D. K. Simanshu, D. V. Nissley, F. McCormick, RAS Proteins and Their Regulators in Human Disease. Cell. 170, 17-33 (2017).
9. R. Ren, Mechanisms of BCR-ABL in the pathogenesis of chronic myelogenous leukaemia. Nat. Rev. Cancer. 5, 172-183 (2005).
10. R.-K. Lin, Y.-C. Wang, Dysregulated transcriptional and post-translational control of DNA methyltransferases in cancer. Cell Biosci. 4, 46 (2014).
11. A. A. Alizadeh et al., Toward understanding and exploiting tumor heterogeneity. Nat. Med. 21, 846-853 (2015).
12. A. Nguyen, M. Yoshida, H. Goodarzi, S. F. Tavazoie, Highly variable cancer subpopulations that exhibit enhanced transcriptome variability and metastatic fitness. Nat. Commun. 7, 11246 (2016).
13. R. J. Taft, K. C. Pang, T. R. Mercer, M. Dinger, J. S. Mattick, Non-coding RNAs: Regulators of disease. J. Pathol. 220 (2010), pp. 126-139.
14. M. Esteller, Non-coding RNAs in human disease. Nat. Rev. Genet. 12, 861-874 (2011).
15. A. J. Minn et al., Distinct organ-specific metastatic potential of individual breast cancer cells and primary tumors. J. Clin. Invest. 115, 44-55 (2005).
16. J. M. Loo et al., Extracellular Metabolic Energetics Can Promote Cancer Progression. Cell. 160, 393-406 (2015).
17. D. N. Cooper, L. P. Berg, V. V Kakkar, J. Reiss, Ectopic (illegitimate) transcription: new possibilities for the analysis and diagnosis of human genetic disease. Ann. Med. 26, 9-14 (1994).
18. A. A. Margolin et al., ARACNE: An Algorithm for the Reconstruction of Gene Regulatory Networks in a Mammalian Cellular Context. BMC Bioinformatics. 7, S7 (2006).
19. H. Goodarzi et al., Metastasis-suppressor transcript destabilization through TARBP2 binding of mRNA hairpins. Nature (2014), doi: 10.1038/nature13466.
20. B. Kim, K. Jeong, V. N. Kim, Genome-wide Mapping of DROSHA Cleavage Sites on Primary MicroRNAs and Noncanonical Substrates. Mol. Cell. 66, 258-269.e5 (2017).
21. D. Ray et al., A compendium of RNA-binding motifs for decoding gene regulation. Nature. 499, 172-177 (2013).
22. Y.-C. T. Yang et al., CLIPdb: a CLIP-seq database for protein-RNA interactions. BMC Genomics. 16, 51 (2015).
23. E. L. Van Nostrand et al., Robust transcriptome-wide discovery of RNA-binding protein binding sites with enhanced CLIP (eCLIP). Nat. Methods. 13, 508-514 (2016).
24. H. Goodarzi et al., Systematic discovery of structural elements governing stability of mammalian messenger RNAs. Nature. 485, 264-268 (2012).
25. S. Memczak et al., Circular RNAs are a large class of animal RNAs with regulatory potency. Nature. 495, 333-8 (2013).
26. P. Sumazin et al., An extensive microRNA-mediated network of RNA-RNA interactions regulates established oncogenic pathways in glioblastoma. Cell. 147, 370-381 (2011).
27. A. Helwak, G. Kudla, T. Dudnakova, D. Tollervey, Mapping the human small non-coding RNA interactome by CLASH reveals frequent noncanonical binding. Cell. 153, 654-65 (2013).
28. T. Fiskaa et al., Distinct Small RNA Signatures in Extracellular Vesicles Derived from Breast Cancer Cell Lines. PLOS ONE. 11 (2016), doi: 10.1371/journal.pone.0161824.
29. W. Zhou et al., Cancer-secreted miR-105 destroys vascular endothelial barriers to promote metastasis. Cancer Cell. 25, 501-515 (2014).
30. S. K. Chakrabortty, A. Prakash, G. Nechooshtan, S. Hearn, T. R. Gingeras, Extracellular vesicle-mediated transfer of processed and functional RNY5 RNA. RNA N. Y. N. 21, 1966-1979 (2015).
31. X. Wu et al., De novo sequencing of circulating small non-coding RNAs identifies novel markers predicting clinical outcome of locally advanced breast cancer. J. Transl. Med. 10, 42-42 (2012).
32. M. D. Giraldez et al., Accuracy, Reproducibility And Bias Of Next Generation Sequencing For Quantitative Small RNA Profiling: A Multiple Protocol Study Across Multiple Laboratories. bioRxiv, 113050 (2017).
33. O. Elemento, N. Slonim, S. Tavazoie, A universal framework for regulatory element discovery across all Genomes and data types. Mol. Cell. 28, 337-350 (2007).

1. A method of diagnosing a subject with a benign, pre-malignant, or malignant hyperproliferative cell comprising:
detecting the presence, absence, and/or quantity of at least one orphan non-coding RNA (oncRNA) or functional fragment thereof in a sample from the subject using one or more probe specific for oncRNA.

2. The method of claim 1, wherein the subject is a human diagnosed with or suspected as having a breast cancer.

3. The method of claim 1, wherein the step of detecting comprises:
a) contacting the sample with one or a plurality of probes specific for the at least one oncRNA or functional fragment thereof, and normalizing the quantity in the sample with a measurement taken from a control sample;
b) contacting the total RNA of the sample to at least one probe complementary to one or a combination of nucleic acid sequences comprising any of the sequences in Tables 1, 2, and/or 3; and/or
c) using a chemiluminescent probe, fluorescent probe, and/or fluorescence microscopy.

4. The method of claim 1 further comprising exposing a sample from a subject to at least one probe specific for one or a combination of oncRNAs chosen from: SEQ ID NO: 1 through SEQ ID NO:201 or one or a combination of oncRNAs that comprise at least 70%, 80%, 85%, 90%, 95%, 96%, 97%, 98%, 99% sequence homology to any nucleic acid of Tables 1, 2, and/or 3.

5. The method of claim 1, wherein the at least one oncRNA is T3p or a functional fragment thereof.

6. The method of claim 1, further comprising correlating the amount of at least one oncRNA or homologous sequence thereof in the sample to the probability or likelihood the subject has a benign, pre-malignant, or malignant growth, relative to a measurement of the amount of at least one oncRNA or homologous sequence thereof in a control sample.

7. The method of claim 1, wherein the benign, pre-malignant, or malignant hyperproliferative cell is from breast tissue.

8. The method of claim 1, wherein the sample comprises:
a) blood or serum from a subject;
b) RNA taken from a culture of cells seeded or inoculated by at least one cell from a subject;
c) human tissue sample comprising a tissue or liquid sample from a plasma, serum or blood draw, brushing, biopsy, or surgical resection of a subject; or
d) a cell that is freshly obtained, formalin fixed, alcohol-fixed and/or paraffin embedded.

9. The method of claim 1 further comprising culturing at least one biopsy from the subject with a culture medium under conditions and for a time period sufficient to grow at least one cell from a subject's breast tissue.

10. The method of claim 1, wherein the step of measuring the quantity of at least one oncRNA or a functional fragment thereof in a sample comprises one or a combination of: digitally imaging a sample; exposing a sample to a known amount of labeled antibody specific for an epitope of oncRNA or a functional fragment thereof, exposing a sample to one or a plurality of dyes of specific for oncRNA or a functional fragment thereof, exposing a sample to at least one labeled probe complementary to a sequence of the oncRNA or a functional fragment thereof, exposing a sample to chromatography, isolating total RNA of a sample and exposing the total RNA to sequencing analysis and/or exposing the sample to mass spectrometry, and, optionally, further comprising analyzing morphology of cells from the sample.

11. The method of claim 1, comprising:
a) contacting one or a plurality of probes specific for the at least one oncRNA or functional fragment thereof with the sample;
b) quantifying the presence, absence, or quantity of the at least one oncRNA or functional fragment thereof in the sample;
c) calculating one or more scores based upon the presence, absence, or quantity of the at least one oncRNA or functional fragment thereof;
d) correlating the one or more scores to the presence, absence, or quantity of the at least one oncRNA or functional fragment thereof, such that, if the amount of the at least one oncRNA or functional fragment thereof is greater than the quantity of the at least one oncRNA or functional fragment thereof in a control sample, the correlating step comprises diagnosing the subject with the benign, pre-malignant, or malignant hyperproliferative cell.

12. A method of detecting a cancer cell in a subject comprising:
detecting whether a oncRNA is present in a sample of the subject by contacting the sample with an amount of one or a combination of probes complementary to one or a combination of oncRNA sequences.

13. The method of claim 12, wherein the step of detecting is preceded by a step of obtaining RNA from the sample of the subject.

14. The method of claim 12 further comprising:
calculating one or more scores based upon the presence, absence, or quantity of one oncRNA and/or a homologous sequence thereof; and
correlating the one or more scores to the presence, absence, or quantity of oncRNA and/or functional fragment thereof, such that, if the amount of oncRNA and/or functional fragment thereof is greater than the quantity of oncRNA and/or functional fragment thereof in a control sample; or, if the amount of oncRNA and/or functional fragment thereof is substantially equal to the quantity of oncRNA and/or functional fragment thereof in a sample taken from a subject known to have cancer then the subject is diagnosed as having cancer.

15. The method of claim 12 further comprising detecting a presence or quantifying two or more oncRNAs chosen from those nucleic acid sequences of Tables 1, 2, and/or 3 or one or combination of nucleic acids sequences that comprise at least 70%, 80%, 85%, 90%, 95%, 96%, 97%, 98%, 99% sequence homology to any of the sequences of Tables 1, 2 and/or 3.

16. The method of claim 12, wherein the sample:
a) is a human tissue sample comprising a tissue from a serum or plasma or blood draw, brushing, biopsy, or surgical resection of the subject;
b) comprises total RNA from a cell that is freshly obtained, formalin fixed, alcohol-fixed and/or paraffin embedded; or
c) is plasma, blood or serum.

17. The method of claim 12, wherein the probe is complementary to an oncRNA sequence that is T3p.

18. A method of treating a subject in need thereof diagnosed with or suspected of having breast cancer, comprising:
(a) contacting one or a plurality of probes specific for one or a combination of oncRNAs and/or a homologous sequence thereof with a sample;
(b) quantifying the presence, absence or amount of oncRNA and/or a homologous sequence thereof in the sample;
(c) calculating one or more scores based upon the presence, absence, or quantity of oncRNA and/or a homologous sequence thereof;
(d) correlating the one or more scores to the presence, absence, or quantity of oncRNA and/or a homologous sequence thereof, such that, if the amount of oncRNA and/or a homologous sequence thereof is greater than the quantity of oncRNA and/or a homologous sequence thereof in a control sample, the correlating step comprises diagnosing a subject with breast cancer; and
(e) administering to the subject a therapeutically effective amount of treatment for the breast cancer.

19. The method of claim 18, wherein the probe is one or a plurality of nucleic acid sequences complementary to a nucleic acid sequence chosen from one or a combination of sequences of Table 1, Table 2 and/or Table 3.

20. The method of claim 19, wherein the one or plurality of probes comprise a fluorophore, a chemiluminescent agent, and/or a quenching agent.

21. A method of diagnosing a subject with a benign, pre-malignant, or malignant hyperproliferative cell comprising:
detecting the presence, absence, and/or quantity of one or more oncRNA or functional fragment thereof in a sample from the subject using one or more probe specific for an oncRNA selected from SEQ ID NO:3, SEQ ID NO: 19, SEQ ID NO:32, SEQ ID NO: 40, SEQ ID NO:41, SEQ ID NO: 79, SEQ ID NO:82, SEQ ID NO:83, SEQ ID NO: 126, SEQ ID NO: 148 and SEQ ID NO: 191.
